(19) United States
(12) Patent Application Publication
(10) Pub. No.: US 2023/0123488 A1
(43) Pub. Date: Apr. 20, 2023

(54) STABLE TOPICAL TETRACYCLINE COMPOSITIONS

(71) Applicant: VYNE THERAPEUTICS INC., Bridgewater, NJ (US)

(72) Inventors: Yohan HAZOT, Rehovot (IL); Tal BERMAN, Rishon Le Ziyyon (IL); Jana ZAILER, Rehovot (IL); Orit AMSALEM, Rehovot (IL); David SCHUZ, Gimzu (IL); Anat LONDON-DRORI, Beer Yakov (IL)

(21) Appl. No.: 16/624,244

(22) PCT Filed: Jun. 19, 2018
(86) PCT No.: PCT/IB2018/000767

§ 371 Date: Dec. 18, 2019

Related U.S. Application Data
(60) Provisional application No. 62/522,610, filed on Jun. 20, 2017.
(60) Provisional application No. 62/685,699, filed on Jun. 15, 2018.

(51) Int. Cl. A61K 9/12; A61K 9/00; A61K 31/65

(52) U.S. Cl. CPC A61K 9/122; A61K 9/0014; A61K 31/65

(57) ABSTRACT

Provided herein are compositions and methods for treating skin disorders, e.g., acne and/or rosacea. In some embodiments, the compositions may comprise carriers which are suitable for stabilizing an unstable active agent, e.g., a tetracycline antibiotic.

### RELATED APPLICATIONS

This application claims the benefit of priority of U.S. Provisional Application No. 62/522,610, filed Jun. 20, 2017, and U.S. Provisional Application No. 62/685,699, filed Jun. 15, 2018, each of which is incorporated herein by reference in its entirety.

### TECHNICAL FIELD

The present disclosure relates to non-emulsion carriers suitable for unstable active agents, compositions comprising the carriers with active agents, and methods of using them.

The disclosure further relates in certain aspects to carriers that are free of surface-active agents and/or that are non-aqueous or substantially or essentially non-aqueous.

The compositions can be essentially or substantially homogenous two-phase carrier compositions. The compositions can comprise a polar phase and a hydrophobic phase. The polar phase can be substantially entrapped or distributed within the hydrophobic phase.

The hydrophobic phase also referred to as ‘entrapment framework’ is capable of holding the polar phase as a sort of quasi-glue. In one or more embodiments, the hydrophobic phase and hydrophilic polar phase form a complex arrangement in which aliquots of liquid polar phase are surrounded by aliquots of hydrophobic phase and the complex arrangement is without the presence of surfactants to bridge and interlink the hydrophobic and polar phases. This type of complex arrangement can form a two-phase homogenous composition, one phase being liquid and the other a fluid solid or semi solid, and once intermixed can be referred to as a “liquisoft” complex.

The liquisoft complex may comprise one or more active pharmaceutical ingredients, which may be fully or partially dissolved or suspended in one of the phases.

Methods of treatment using the disclosed compositions are also provided.

### BACKGROUND

Oral Tetracycline antibiotics are useful for the treatment of infections of the urinary tract, the respiratory tract, and the intestines, acne and rosacea. However, the systemic delivery of tetracycline antibiotics is associated with adverse side effects, including diarrhea, abdominal cramps, and dizziness.

Topical formulations of tetracycline antibiotics are extremely challenging to develop, as these active agents are highly susceptible to degradation by a wide range of pharmaceutical carriers and excipients. Tetracycline antibiotics are also sensitive to moisture, temperature, and light. Tetracycline antibiotics are generally unstable whether suspended in a carrier composition or dissolved. The challenge of topically administering tetracycline antibiotics includes the identification of a carrier system in which a tetracycline antibiotic remains stable for a sufficiently long period of time for product distribution, storage at a pharmacy, and therapeutic use by a patient. Additional challenges include providing a carrier that can partially or completely dissolve an active agent, and in particular a tetracycline antibiotic, without significant degradation. It is also challenging to develop a delivery system that allows an active agent, e.g., a tetracycline antibiotic, to penetrate into the skin or mucosa whilst avoiding degradation and preventing the active agent from reaching the blood system in significant or substantial amounts to avoid or minimize potential systemic side effects.

Methods for treating a subject suffering from acne, with a substituted tetracycline compound are known. However, those disclosed methods neither disclose suitable compositions, their components and ratios nor suggest the use of a two-phase homogenous, substantially waterless or waterless composition without surfactants comprising hydrophobic and polar phases, wherein the tetracycline is solubilized and remains stable in the composition. Waterless, surfactant-based foam compositions comprising tetracycline antibiotics, at least one oil, and a propellant are further known. In contrast, the compositions described herein do not use surfactants to bridge and link the hydrophobic and polar phases. Surfactant-free foam formulations of tetracycline antibiotics comprising a hydrophobic solvent, a fatty alcohol and or a fatty acid, and a wax are also known. Such formulations generally exclude hydrophilic or polar solvents. In those formulations, the tetracycline antibiotics are suspended.

In contrast, the compositions described herein comprise a liquid polar solvent, which may allow tetracycline antibiotics to be fully or partially dissolved in the polar solvent, and the solvent may be in turn evenly distributed within a hydrophobic phase. Some tetracycline antibiotic compositions relate to a suspended teracycline antibiotic that is conjugated to a stabilizing particle (e.g., gold particles). Other tetracycline compositions involve a tetracycline stabilized by a metal salt and an anti-oxidant sulfite compound in high amounts of ethanol. Such compositions are very different from the compositions described herein which comprise a fully or partly solubilize tetracycline that is not conjugated and is not stabilized by a metal cation and/or an anti-oxidant sulfite compound. Additional known tetracycline antibiotics formulations relate to polymeric hydrocarbon gel-based compositions of micronized tetracycline antibiotics, wherein the tetracycline is suspended in the composition. As mentioned above, the herein disclosed compositions advantageously afford a tetracycline formulation in which the tetracycline antibiotics may be fully or partially dissolved in the polar solvent, where it remains stable. Further polymer-based tetracycline antibiotics include occlusive dressing compositions (e.g., an elastomeric gel of a plasticizing oil and a block copolymer phase), hydrogel copolymers, and other cross linkable polymers. Such polymer-based compositions include polyester copolymers, styrene/ethylene/ butylene/propylene block polymers and cross linkable polymers. They do not include a silicone-based polymer, nor do they provide a polar-hydrophobic two-phase homogeneous liquisoft complex including a wax and free of surfactants, which solubilizes and stabilizes the active ingredient. An Emu oil and a succinic acid based antibiotic compositions are further known. Glycol and silicone based emulsions with surfactants are also known. Other emulsions known in the art generally require solid silica particulate dispersant to stabilize oil in polar emulsion or polar in oil emulsion. None of the above-mentioned art discloses, non-emulsion tetracycline carriers that comprise a polar phase that is substantially trapped in a hydrophobic phase without the presence of surface active agents or solid silica particulate or polymers, or metal salt or sulfite compounds and remain stable and substantially homogenous despite the pressure for the hydrophilic and hydrophobic phases to separate.

### SUMMARY

Provided herein, in one or more embodiments, are carriers for active agents, compositions comprising such carriers with one or more active agents, and methods for manufacture and use of the carriers. The carriers are well suited for unstable active agents. In one or more embodiments, the unstable active agents are tetracycline antibiotics. In one or more embodiments, provided herein are compositions comprising a tetracycline antibiotic. In one or more embodiments, the tetracycline antibiotic is fully solubilized within the composition. In some embodiments, the tetracycline antibiotic is partially solubilized in the composition. In some embodiments, the carrier composition is capable of maintaining physical stability and also providing an environment in which the tetracycline antibiotic displays chemical stability. The carriers may be used for other unstable active ingredients and for stable ones also. Chemical and physical stability can be maintained for prolonged periods of time such that the composition is suitable for pharmaceutical use.

The liquisoft carriers and compositions are suitable for topical application to the skin and mucosa and for example can deliver a drug, such as a tetracycline antibiotic (e.g., a minocycline or a doxycycline) topically to the skin or mucosa, with low, almost no, or negligible systemic penetration and thereby avoid unwanted systemic effects following oral administration.

The carriers and compositions described herein have the potential to be adapted for multiple uses, e.g., with different types or combinations of active agents and/or for different therapeutic indications and in different delivery forms or modes. In some aspects, the combination is of an unstable active agent with a stable active agent. In some aspects, the combination is of two or more unstable active agents. In some aspects, one active agent is within the hydrophobic phase and the other is within the polar phase. In some aspects, the combination is of two or more incompatible active agents. In some aspects, the two or more active agent are within the hydrophobic phase or within the polar phase, or between the hydrophobic and polar phases.

In some embodiments, the compositions are ointments. In some embodiments, they are gels. In some embodiments, they are oleaginous. In some embodiments, they are foams.

Thus, provided herein are non-emulsion carriers composition suitable for unstable active agents. Tetracycline antibiotics are known to be very unstable even when suspended and especially so if they are solubilized or dissolved. As will be appreciated by one skilled in the art, unstable active agents tend to breakdown when in solution. So new delivery forms of stabilized solubilized or dissolved tetracycline antibiotics are herein revealed, which can also be used for other active agents.

The carriers may comprise two phases, one of which is substantially entrapped or held in the other phase without the use of standard or customary surfactants, emulsifiers, and/or without co-surfactants. In some alternative embodiments co-surfactants may be used without surfactants.

A liquisoft complex may have two primary phases an entrapment framework and an entrapped phase. In some embodiments the entrapment framework is hydrophobic and comprises oil and wax, or oil, wax and a thickened silicone, or wax and thickened silicone and substantially or primarily entraps the polar solvent. The framework can comprise a general framework or a multi-level framework. In the general framework the interspaces may in some aspects comprise oil or oil and wax, or an oil, a wax and a thickened silicone, or a wax and a thickened silicone, in some aspects polar solvent and in some aspects some interspaces having oil or oil and wax and some interspaces having polar solvent. In the multi-layer framework on one level some oil wax covers some polar solvent, and on another level some other aliquot of polar solvent covers part or all that oil wax and on another level some or all of that aliquot of polar solvent is covered with oil wax and so on.

In one or more embodiments, the composition is a liqisoft complex in which one of the two phases is substantially or primarily covered or entrapped by the other phase. In one or more embodiments, the liquisoft complex comprises a polar phase that is entrapped by a hydrophobic phase, also called entrapment framework. In one or more embodiments, the liquisoft complex comprises a hydrophobic phase that is entrapped by a polar phase. In one or more embodiments, the liquisoft complex comprises a polar phase that is entrapped by a hydrophobic phase, wherein said hydrophobic phase is further entrapped or covered by a polar phase. In one or more embodiments, the liquisoft complex comprises a multi-layered structure comprising two or more layers of polar phase and hydrophobic phase, wherein each layer entraps the layer beneath it.

The carriers are physically stable and are substantially or effectively homogenous, appearing as a single phase to the naked eye. Under the microscope, however, one phase is viewed as being, in one or more embodiments, entrapped, or covered in the other and substantially uniformly distributed. In one or more embodiments, the entrapment framework, or liquisoft complex comprises a hydrophobic phase or solvent comprising a wax and the entrapped, or primarily covered phase comprises a hydrophilic solvent such as a polar solvent or a polyol. In some embodiments, the polyol is or comprises a triol. In some embodiments, the polar or hydrophilic solvent is a polyol, such as a triol. In some embodiments, the triol is or comprises glycerin (glycerol). In some embodiments, the polyol is a combination of triols or is a combination of a triol and a diol. In some embodiments, the entrapment framework, or covering framework is a hydrophobic phase. In some embodiments, the hydrophobic phase comprises a wax and an oil. In some embodiments, the hydrophobic phase comprises a wax, an oil and a silicone. In some embodiments the silicone is a thickened silicone. In some embodiments, the hydrophobic phase comprises a wax and a thickened silicone.

In some embodiments, the composition is free of, essentially free of, or substantially free of one or more of surface active agents, polymeric agents, foam adjuvants, solid particles, metal salts, metal cations, sulfite compounds, sulfite anti-oxidants, short chain alcohols, dehydrating agents, antioxidants, preservatives, aromatic alcohols, diols, short chain monohydric alcohols, an emu oil, succinic acid, and solid particulate matter other than wax.

In some embodiment, the composition is free of surface active agents, or is free of polymeric agents, or is free of polyester copolymers, or is free of styrene/ethylene/ butylene/propylene block polymers, or is free of cross linkable polymers, or is free of a gelled mineral oil, or is free of a Versagel, or is free of foam adjuvants, or is free of solid particles, or is free of metal salts, or is free of metal cations, or is free of sulfite compounds, or is free of sulfite anti-oxidants, or is free of short chain alcohols, or is free of dehydrating agents, or is free of antioxidants, or is free of preservatives, or is free of aromatic alcohols, or is free of diols, or is free of short chain monohydric alcohols, or is free of an emu oil, or is free of succinic acid, or is free of solid particulate matter at room temperature other than wax and or other than solid fatty acid and or solid fatty alcohol at room temperature, or is free of any two or more thereof.

In some embodiments, and without being bound by any theory, the entrapment framework has a sufficient strength such as a viscous strength to retain the solvent entrapped, or covered within its scope in a substantially homogeneous distribution.

In one or more embodiments the liquisoft complex may be a soft composition wherein the composition has a soft gel or ointment-like nature, which can provide some fluiditity and/or reduced friction that can aid the provision of an improved skin feeling and or a smoother skin feeling and or lubrication. In some embodiments the soft composition comprises a thickened silicone and a wax. In some embodiments the soft composition comprises a thickened silicone and a wax and an oil.

In some embodiments, the wax has a melting point (or if a mixture of waxes an average melting point) above 20° C., above 25° C., above 30° C., above 35° C., above 40° C., above 45° C., above 50° C., above 55° C., above 60° C., above 65° C., or above 75° C. Wherever the term “a wax” is used throughout the specification, unless expressly stated otherwise, it can mean a single wax or a mixture of waxes. In some embodiments, the wax is a hydrocarbon-based wax. In some embodiments, the wax is or comprises a petroleum derived wax or a mineral wax. In some embodiments, the wax is substantially free of, essentially free of, or free of wax esters. In some embodiments the wax is a combination of waxes. In some embodiments one wax provides smaller particles or crystals and the other larger ones. In some embodiments, the wax is substantially free of, essentially free of, or free of aliphatic esters and/or diesters.

In one or more embodiments, provided herein is a liquisoft complex carrier comprising: a polar phase; a hydrophobic phase comprising a wax, or combinations of two or more waxes having a melting point above 25° C. and a hydrophobic solvent, wherein in one aspect the polar phase is substantially homogeneously mixed with the hydrophobic phase and in another aspect the polar phase is substantially entrapped within the hydrophobic phase, also called an entrapment framework. In a further aspect the entrapment framework is complex and comprises multi layers or levels. In some embodiments the hydrophobic phase comprises an oil, in others it comprises a thickened silicone and in certain embodiments both. In some embodiments the wax has a melting point above 37° C. In one or more embodiments it comprises an active pharmaceutical agent. In one or more embodiments it is in an effective pharmaceutical concentration for topical application to the skin or mucosa to treat a skin disorder involving a bacterial infection and or inflammation, such as acne or rosacea. In one or more embodiments the liquisoft complex is surfactant free. In one or more embodiments it is substantially, essentially or free of water other than water that is associated with the active pharmaceutical agent.

In one or more embodiments, a topical tetracycline antibiotic composition is provided, comprising a polar phase and an entrapment framework comprising a hydrophobic phase, wherein the two phases, when mixed, form a substantially homogenous composition. In one or more embodiments, the polar phase is physically held and stabilized by the entrapment framework and is distributed, or entrapped homogenously or substantially so within the entrapment framework. In one or more embodiments, the tetracycline antibiotics is solubilized or dissolved within the polar phase. In one or more embodiments, the tetracycline antibiotic is chemically stable within the composition, displaying only a low or slow rate of tetracycline breakdown making it possible for the composition to be stored for a long term (e.g. for 3 or 6 or 9 or 12 or 15 or 18 or 21 or 24 months) at either 5° C. or room temperature and applied for pharmaceutical use and for the active agent to still provide therapeutic efficacy after administration.

In one or more embodiments, there is provided a method of stabilizing doxycycline in a glycerol composition, wherein the composition is a metal salt free, anti-oxidant free composition, wherein the concentration of doxycycline in glycerol is from 1 to 25%, and the concentration of doxycycline in the total composition is from about 0.1 to 5%. In one or more embodiments, the solubilized concentrated doxycycline is more stable in the two-phase compositions described herein than it is in a single-phase polar-solvent based composition. In one or more embodiments, the doxycycline solubilized in the polar phase of the two-phase compositions described herein is more stable than a doxycycline solubilized in a single-phase polar-solvent based composition. In one or more embodiments, the two-phase compositions described herein retain more of the doxycycline initially present in the composition as compared to the amount of doxycycline retained in a single-phase polar-solvent based composition. In one or more embodiments, provided herein are two-phase homogenous compositions comprising a first phase being a polar phase and a second phase; wherein an active agent (e.g. doxycycline) is solubilized in the first polar phase; wherein the first polar phase is dispersed in the second phase and wherein the active agent remains stable in the composition. In one or more embodiments, provided herein are two-phase homogenous compositions comprising a polar phase and a hydrophobic phase; wherein an active agent (e.g. doxycycline) is solubilized in the polar phase; wherein the polar phase is dispersed in the hydrophobic phase and wherein the active agent remains stable in the composition. In one or more embodiments, provided herein are methods of stabilizing an active agent in a two-phase homogenous composition comprising a polar phase and a hydrophobic phase; wherein the active agent (e.g., doxycycline) is solubilized and concentrated in the polar phase and wherein the polar phase is dispersed in or entrapped within the hydrophobic phase. In one or more embodiments, provided herein are methods of stabilizing a doxycycline; wherein the doxycycline is concentrated in a polar phase (e.g., glycerol), and wherein the polar phase is dispersed in an insoluble phase; wherein the doxycycline is more stable as compared to the same amount of doxycycline solubilized in the polar phase alone.

In some embodiments, the compositions are substantially free of, essentially free of, or free of substances that could irritate skin or mucosa (referred to herein as “irritants”), in particular when skin or mucosa is damaged, inflamed, and or broken. In some embodiments, the compositions are substantially free of, essentially free of, or free of such irritants especially when the compositions are intended for, e.g., children or other patients who are less likely to comply with treatment. In some embodiments, the composition is substantially free of, essentially free of, or free of one or more of surfactants, salts, short chain alcohols, aromatic alcohols, acids, and/or bases, or contain only amounts that do not produce skin irritation and/or dry skin with repeated use. Thus, in some embodiments, provided herein are carriers and compositions and methods of topical administration that do not impair the integrity of the skin, e.g., by causing skin irritation and/or dry skin. In some embodiments, the compositions and methods of topical administration allow for high patient compliance as the compositions are easy and comfortable to apply.

In one or more embodiments, provided herein is two phase carrier composition, the carrier composition comprising an organic polar phase comprising at least one polyol; and an organic hydrophobic phase comprising a) at least one wax having a melting point of above 25° C. and b) at least one hydrophobic liquid solvent; or at least one hydrophobic solvent and at least one thickened silicone; or at least one thickened silicone, wherein the polar phase is essentially or substantially homogenously distributed within the hydrophobic phase.

Thus, in one or more embodiments, provided herein is a topical two-phase homogenous carrier composition, the carrier composition comprising
i) an organic polar phase comprising at least one polyol; and
ii) an organic hydrophobic phase comprising a) at least one wax having a melting point above 37° C. b) at least one hydrophobic liquid solvent; and c) optionally at least one thickened silicone;
wherein the polar phase is entrapped within the hydrophobic phase, and wherein the composition is free of a surfactant.

In one or more embodiments, provided herein is a topical two-phase homogenous carrier composition, the carrier composition comprising
i) an organic polar phase comprising at least one polyol; and
ii) an organic hydrophobic phase without a hydrophobic solvent comprising a) at least one wax having a melting point above 37° C. and b) at least one thickened silicone;
wherein the polar phase is entrapped within the hydrophobic phase, and wherein the composition is free of a surfactant.

In one or more embodiments, provided herein is a two phase homogenous carrier composition, the carrier composition comprising
i) about 1% to about 70% by weight of the composition of an organic polar phase comprising at least one polyol; and
ii) an organic hydrophobic phase comprising a) about 5% to about 50% by weight of the composition at least one wax having a melting point of above 37° C. and b) about 10% to about 75% by weight of the composition of at least one hydrophobic liquid solvent;
wherein the polar phase is entrapped within the hydrophobic phase, and wherein the composition is free of a surfactant.

In one or more embodiments, provided herein is a two phase homogenous carrier composition, the carrier composition comprising
i) about 1% to about 70% by weight of the composition of an organic polar phase comprising at least one polyol; and
ii) an organic hydrophobic phase comprising a) about 5% to about 50% by weight of the composition at least one wax having a melting point of above 37° C., b) about 10% to about 75% by weight of the composition of at least one hydrophobic liquid solvent and c) about 5% to about 40% by weight of the composition of at least one thickened silicone;
wherein the polar phase is entrapped within the hydrophobic phase, and wherein the composition is free of a surfactant.

In one or more embodiments, provided herein is a two phase homogenous carrier composition, the carrier composition comprising
i) about 1% to about 70% by weight of the composition of an organic polar phase comprising at least one polyol; and
ii) an organic hydrophobic phase comprising a) about 5% to about 50% by weight of the composition at least one wax having a melting point of above 37° C. and b) about 25% to about 85% by weight of the composition of at least one thickened silicone;
wherein the polar phase is entrapped within the hydrophobic phase, and wherein the composition is free of a surfactant.

In some embodiments, the composition is essentially free of surface active agent. In some embodiments, the composition is substantially free of surface active agents. In one or more embodiments, the composition is substantially waterless. In some embodiments, the composition is essentially waterless. In some embodiments, the composition is waterless. In some embodiments, the tetracycline antibiotic is dissolved in the composition. In other embodiments, the tetracycline antibiotic is partially dissolved and partially suspended in the composition. In some embodiments, the tetracycline antibiotic is dissolved in the polar phase. In some embodiments, the tetracycline antibiotic is partly dissolved in the polar phase (e.g., at least 50, 60, 70, 80, or 90% dissolved) and partly suspended in the hydrophobic phase. In some embodiments, the tetracycline antibiotic is mostly dissolved in the polar phase (e.g., at least 90% dissolved). In some embodiments, the tetracycline antibiotic is almost entirely dissolved (e.g., at least 98, 98.5, 99, 99.2, 99.4, 99.6, 99.8 or 99.9% dissolved). In some embodiments, it is fully dissolved (e.g., >99.9%). In some embodiments, the polar phase comprises a first tetracycline antibiotic and the hydrophobic phase comprises a second tetracycline antibiotic. In some embodiments, the polar phase comprises a tetracycline antibiotic and the hydrophobic phase comprises an additional active agent that may or may not be a tetracycline antibiotic. In some embodiments, the polar phase comprises at least two tetracycline antibiotics and the hydrophobic phase comprises another active agent and/or a different tetracycline antibiotic. In some embodiments, the polar phase comprises a first active agent and the hydrophobic phase comprises a second active agent.

In one or more embodiments, a carrier described herein comprises at least one wax which allows or facilitates the hydrophobic solvent/component and hydrophilic or polar solvent to form an essentially or substantially homogenous or uniform composition. For example, in an embodiment, the polar phase of the carrier comprises a liquid polyol, and the entrapment framework comprises a paraffin wax that is solid at 37° C. and the tetracycline antibiotic is dissolved or substantially dissolved in the polyol. In one or more embodiments, the polar phase comprising the solubilized tetracycline is distributed, or entrapped within the entrapment framework. These compositions are able to provide physically and chemically stable tetracycline compositions.

As an example, the stability of tetracycline antibiotics in a composition can be assessed, e.g., by visually examining the appearance of the compositions. Alternatively, or in addition, the stability can be assessed by analyzing breakdown markers, for example, the amount or rate of appearance of tetracycline degradant epimer-4 (also referred as “4-epi”) breakdown product or of the total impurities, or of the color change of the composition. In one or more embodiments, tetracycline antibiotic compositions described herein are stable when assessed using one or more of the parameters described herein. It has been found that the rate of appearance of 4-epi breakdown product may depend on the type of tetracycline antibiotic and the carrier into which the tetracycline antibiotic is formulated. In one or more embodiments, the rate of appearance per month of 4-epi breakdown product in a tetracycline antibiotic is reduced by using a carrier comprising polar and hydrophobic phases in a substantially waterless and/or substantially surface active agent free composition and may be in some embodiments further reduced in a surface active agent free and essentially water free compositions. In one or more embodiments, such a composition is formulated without using known techniques to reduce breakdown of adding substantial amounts of a divalent magnesium cation and/or effective amounts of antioxidant such as a sulfite, and/or by providing a dehydrating agent, such as ethyl acetate, acetic anhydride and ethanol, and/or by providing high concentrations of preservatives. In one or more embodiments, the carrier or composition is free of a metal cation such as magnesium and or an antioxidant, and or a dehydrating agent. In some embodiments, the carrier or composition is essentially free thereof. In some embodiments, the carrier or composition is substantially free thereof. In some embodiments, the average rate of appearance of 4-epi breakdown product is less than about 0.7% per month at 40° C., or less than about 0.2% per month at 25° C. In some embodiments, the average rate of appearance of 4-epi breakdown product is less than about 1% per month, or less than about 0.9% per month, or less than about 0.8% per month, less than about 0.7% per month, or less than about 0.6% per month, or less than about 0.5% per month when incubated at 40° C. In some embodiments, the average rate of appearance of 4-epi breakdown product is less than about 0.4% per month, or less than about 0.3% per month, or less than about 0.2% per month, less than about 0.1% per month, or less than about 0.07% per month or less than about 0.05% per month at 30° C., 25° C., or 5° C.

In one or more embodiments, the composition is a non-emulsion. In one or more embodiments, the composition is a non-classic dispersion. In one or more embodiments, the composition is a non-classic gel comprising no water with no gelling agent or a de-minimis amount of a gelling agent. In one or more embodiments, the composition is not an oil gel. In one or more embodiments, the composition is a pseudo-dispersion. In one or more embodiments, the composition is a two phase substantially homogenous composition. In one or more embodiments, the composition is a two phase essentially homogenous composition. In one or more embodiments, the composition is a non-classical ointment in which a polar phase is distributed essentially uniformly in an entrapment framework. In one or more embodiments, the composition is a low entropy composition in which polar and hydrophobic phases -- facilitated by a wax and optionally a thickened silicone -- present an essentially uniform distribution of the polar solvent in the hydrophobic phase.

In one or more embodiments, the composition is substantially free, or essentially free, or free of a short chain monohydric alcohol having up to 6 carbon atoms in its carbon chain, such as ethanol.

In certain embodiments, the tetracycline antibiotic is a doxycycline. In certain embodiments, the tetracycline antibiotic is a minocycline.

In some embodiments, the polar phase is present in the composition at about 5% to about 85% by weight of the composition, at about 7% to about 25% by weight of the composition, at about 10% to about 50% by weight of the composition, at about 15% to about 35% by weight of the composition, at about 25% to about 45% by weight of the composition, at about 12% to about 47% by weight of the , at about 45% to about 55% by weight of the composition, at about 55% to about 65% by weight of the composition, at about 65% to about 75% by weight of the composition, or at about 75% to about 85% by weight of the composition. In one or more embodiments, the composition comprises a polar solvent in a concentration of about 5% to about 35% by weight of the composition. For e.g. about 5% to about 10% by weight, about 10% to about 15% by weight, about 15% to about 20% by weight, about 20% to about 25% by weight, about 25% to about 30% by weight or about 30% to about 35% by weight of the composition. In one or more embodiments the composition comprises a polar solvent in a concentration of less than about 80% by weight of the composition, or less than about 70% by weight of the composition, or less than about 60% by weight of the composition, or less than about 50% by weight of the composition, or less than about 40% by weight of the composition, or less than about 30% by weight of the composition, or less than about 20% by weight of the composition. In one or more embodiments, the polar phase comprises one or more polyols. In some embodiments, the polyol comprises a diol. In some embodiments, the polyol does not include a diol. In some embodiments, the polyol comprises a triol.

In some embodiments, the entrapment framework is present in the composition at about 25% to about 90% or about 35% to about 85% by weight of the composition. In some embodiments, the entrapment framework is present in the composition at about 20% to about 95% by weight of the composition. In some embodiments, it is between about 25% to about 85% or between about 25% to about 80% by weight of the composition. In some embodiments, it is between about 30% to about 90%, between about 33% to about 87%, between about 35% to about 85%, between about 38% to about 82%, or between about 40% to about 80%, or between about 50% to about 85%, or between about 60% to about 80%, or between about 65% to about 90%, or between about 70% to about 95% by weight of the composition. In some embodiments, it is at least about 30%, at least about 40%, at least about 50%, at least about 60%, at least about 65%, or at least about 70% by weight of the composition. In one or more embodiments, the hydrophobic phase is about 70%, about 72%, about 74%, about 76%, about 78%, about 80%, about 82%, about 84%, about 86%, about 88%, about 90%, or about 92% by weight of the composition. In some embodiments, it is less than about 95% by weight of the composition, or less than about 90%, or less than about 85%, or less than about 80%, or less than about 75%, or less than about 70%, or less than about 65%, or less than about 60%, or less than about 55%, or less than about 50%, or less than about 45%, or less than about 40% by weight of the composition.

In some embodiments, the entrapment framework comprises a hydrophobic solvent and a wax or a hydrophobic solvent and a wax and a thickened silicone, or a wax and a thickened silicone. In some embodiments, the hydrophobic solvent is an oil. In some embodiments, the oil is a hydrocarbon oil. In some embodiments, the oil is a mineral oil. In some embodiments, the oil is a petrolatum. In some embodiments, the entrapment framework comprises a liquid fatty acid. In yet another embodiment, the entrapment framework comprises a liquid fatty alcohol. In other embodiments, the carrier and composition are free of a solid fatty alcohol and/or free of a solid fatty acid. In some embodiments, the carrier or composition is free of fatty alcohols having a carbon chain of C 14 to C22. In some embodiments, the carrier or composition is free of fatty acids having a carbon chain of C15 to C24. In some embodiments, the composition is free of stearyl alcohol and or cetyl alcohol and or stearic acid.

In some embodiments, the wax is a hydrocarbon-based wax. In some embodiments, the wax is a paraffin wax. In some embodiments, the wax is a solid fatty acid. In other embodiments, the wax is a solid fatty alcohol. In some embodiments, a liquid wax may be used in combination with a solid wax. Jojoba oil and oleic acid are examples of a liquid wax.

In some embodiments, the wax is a paraffin wax and the polyol is a triol. In some embodiments, the wax is a paraffin wax and the polar phase of the composition described herein further comprises a monohydric alcohol. In some embodiments, the composition comprises a paraffin wax and propanol. In one or more embodiments embodiment, the carrier or composition may be presented as an oleaginous composition; a non-emulsion; a non-classic dispersion; an ointment, a gel; or a foam. Herein disclosed are compositions formulated to provide stability of unstable active agents like tetracycline antibiotics and may be applied topically onto skin or mucosa surfaces.

### BRIEF DESCRIPTION OF DRAWINGS

FIG. 1 is a microscopic image demonstrating the homogenous entrapment of glycerin in a composition comprising glycerin (20%), paraffin wax 57-60 (10%) and mineral oil (to 100%); the glycerin was dissolved and dyed using FD&C yellow 6 color that dissolves and dyes only polar solvents such as glycerin;

FIG. 2 is a microscopic image (X200) of a doxycycline formulation (IDD201) comprising 20% w/w glycerin, 40% w/w stearyl alcohol and 37.67% mineral oil, following 3 months incubation at 5° C.; samples were heated to 60° C. prior to microscopic evaluation;

FIGS. 3A-3B are dot plots demonstrating w/w % doxycycline content (FIG. 3A) and progressive w/w % 4-epimer accumulation over time (FIG. 3B) of a doxycycline formulation (IDD201) comprising 20% w/w glycerin, 40% w/w stearyl alcohol and 37.67% mineral oil;

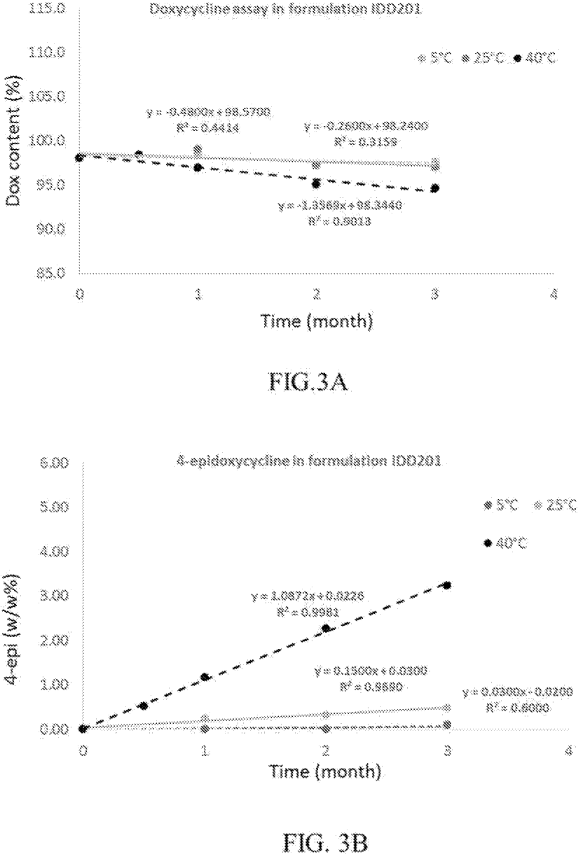

FIGS. 4A-4B are microscopic images (X200) of a doxycycline formulation (IDD203) comprising 20% w/w glycerin, 40% w/w paraffin wax 57-60 and 37.67% mineral oil, following 2 months incubation at 5° C. without (FIG. 4A) or with heating to 60° C. (FIG. 4B) prior to microscopic evaluation;

FIG. 4C is a transparent glass vial of a doxycycline formulation (IDD203) comprising 20% w/w glycerin, 40% w/w paraffin wax 57-60 and 37.67% mineral oil, at T0 at room temperature;

FIGS. 5A-5B are microscopic images (X200) of a placebo formulation comprising 20% w/w glycerin, 40% w/w paraffin wax 57-60 and 37.67% mineral oil, following 2 months incubation at 5° C. without (FIG. 5A) or following heating to 60° C. prior to microscopic evaluation (FIG. 5B);

FIGS. 6A - 6B are dot plots demonstrating w/w % doxycycline content (FIG. 6A) and progressive w/w % 4-epimer accumulation over time (FIG. 6B) of a doxycycline formulation (IDD201) comprising 20% w/w glycerin, 40% w/w stearyl alcohol and 37.67% mineral oil;

FIG. 7 is a dot plot demonstrating w/w % 4-epimer accumulation over time in formulation IDD203 and Composition 1; and

FIG. 8 is a bar graph demonstrating doxycycline penetration within layers of porcine skin, i.e., stratum corneum 1 and 2 (i.e., external layer of stratum corneum and internal layer of sratum corneum), viable skin (i.e., epidermis and dermis) and receiver compartment (designated as receptor fluid).

### DETAILED DESCRIPTION

Unless otherwise defined, all technical and scientific terms used herein have the same meaning as commonly understood by one of ordinary skill in the art to which this disclosure relates. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety including specifically with reference to the excipients and ingredients and amounts. To the extent the specification conflicts with any of the incorporated references, the specification will control. All ranges disclosed herein include the endpoints. The use of the term “or” shall be construed to mean “and/or” unless the specific context indicates otherwise.

There are provided herein liquisoft complex carriers, compositions and their uses and methods of manufacture.

In some embodiments, the entrapment framework comprises a wax and a hydrophobic solvent. In some embodiments, entrapment framework comprises a wax, a hydrophobic solvent and a thickened silicone. In some embodiments, the entrapment framework comprises a wax and a thickened silicone. In some embodiments, the entrapment framework comprises a wax without a hydrophobic solvent. In some embodiments the entrapment framework is without oil. In some embodiments the framework is with fatty alcohols and or acids instead of wax and can be with or without oil.

In some embodiments the polar phase comprises a polyol. Polyols are organic substances that contain at least two hydroxy groups in their molecular structure. In one or more embodiments, the polar solvent contains a diol (a compound that contains two hydroxy groups in its molecular structure), such as propylene glycol (e.g., 1,2-propylene glycol and 1,3-propylene glycol), butanediol (e.g., 1,4-butaneediol), butanediol (e.g., 1,3-butaneediol and 1,4-butenediol), butynediol, pentanediol (e.g., 1,5-pentanediol), hexanediol (e.g., 1,6-hexanediol), octanediol (e.g., 1,8-octanediol), neopentyl glycol, 2-methyl-1,3-propanediol, diethylene glycol, triethylene glycol, tetraethylene glycol, dipropylene glycol and dibutylene glycol. In one or more embodiments, the polar solvent contains a triol (a compound that contains three hydroxy groups in its molecular structure), such as glycerin and 1,2,6-Hexanetriol. Additional examples of polar solvents include polyols, such as glycerol (glycerin), propylene glycol, hexylene glycol, diethylene glycol, propylene glycol n-alkanols, terpenes, di-terpenes, tri-terpenes, terpen-ols, limonene, terpene-ol, 1-menthol, dioxolane, ethylene glycol, other glycols, alkanols, such as dialkylamino acetates, and admixtures thereof, dimethyl isosorbide, ethyl proxitol, dimethylacetamide (DMAc) and alpha hydroxy acids, such as lactic acid and glycolic acid. According to still other embodiments, the polar solvent is a polyethylene glycol (PEG) or PEG derivative that is liquid at ambient temperature, including PEG200 (MW (molecular weight) about 190-210 kD), PEG300 (MW about 285-315 kD), PEG400 (MW about 380-420 kD), PEG600 (MW about 570-630 kD) and higher MW PEGs such as PEG 4000, PEG 6000 and PEG 10000 and mixtures thereof. Yet, in additional embodiments, the polar solvent is an aprotic polar solvent, such as dimethyl sulfoxide (DMSO), dimethylformamide (DMF), acetonitrile, acetone, methyl ethyl ketone, 1,4-Dioxane and tetrahydrofuran (THF). Additional non-limiting examples include N-methylpyrrolidone, pyridine, piperidine, dimethyl ether, hexamethylphosphorotriamide, dimethylformanide, methyl dodecyl sulfoxide, N-methyl-2-pyrrolidone and 1-methyl-2-pyrrolidinone) and azone (1-dodecylazacycloheptan-2-one). Many polar solvents, for example propylene glycol, glycerin, DMSO, and azone possess the beneficial property of a dermal, transdermal or trans-mucosal drug delivery enhancer. In one or more embodiments, the polar solvent is a dermal, transdermal or trans-mucosal drug delivery enhancer. Many polar solvents, for example propylene glycol and glycerin, possess the beneficial property of a humectants. In one or more embodiments, the polar solvent is a humectant.

In some embodiments the carrier comprises an unstable active agent. In some embodiments the carrier comprises a stable active agent. In some embodiments the carrier can comprise two or more active agents, for example two stable or two unstable active agents or one stable and one unstable active agent. In some embodiments one of the active agents is in one phase and the agent is in the other phase. An aspect of the invention provides such carrier compositions wherein the unstable active agent comprises a tetracycline antibiotic. In some embodiments the tetracycline antibiotic is solubilized or dissolved in the polar phase.

Tetracycline antibiotics are known to be very unstable active agents that are prone to degrade in a wide range of commonly used pharmaceutical excipients. In part because of this instability, tetracycline antibiotic compositions are generally provided in oral solid dosage forms or in hydrophobic compositions wherein the tetracycline antibiotics is suspended. Achieving tetracycline antibiotic formulations in which the tetracycline antibiotic is solubilized and is chemically stable is a major challenge since they are more likely to breakdown when in solution than when they are suspended.

Provided herein are physically stable compositions in which the tetracycline is solubilized or dissolved and maintains chemical stability for prolonged time. Also provided are carrier compositions providing for an increased penetration and efficacy of the tetracycline antibiotics.

According to one aspect of the invention, a composition described herein is a tetracycline dual phase composition comprising an organic hydrophilic or polar phase entrapped within an organic hydrophobic or a polar phase. The tetracycline antibiotic may be partially or fully solubilized within the composition. In some embodiments, the compositions are physically stable with no visible phase separation for at least 3, 5, or 6 months when stored at 40° C. In some embodiments, the compositions are physically stable with no visible phase separation for at least 3, 4, 5, 6, 7, 8, 9, 10, 11, or 12 months when stored at 30° C. In some embodiments, the compositions are physically stable with no visible phase separation for at least 3, 4, 5, 6, 7, 8, 9, 10, 11, 12, 15, 18, 21, or 24 months when stored at 25° C. In some embodiments, the compositions are physically stable with no visible phase separation for at least 3, 4, 5, 6, 7, 8, 9, 10, 11, 12, 15, 18, 21, 24, or 36 months when stored at 5° C. In further embodiments, the compositions are chemically stable and accumulate the 4-epi degradant product at below the acceptable levels, with little or no color change, over the time periods at the temperatures mentioned above. In one or more embodiments, a composition comprising a tetracycline antibiotic remains physically and/or chemically stable, as assessed by accumulation of the 4-epi degradant product, for a sufficiently long period of time to allow for, e.g., packaging and storing the product, product distribution, storage at a pharmacy, and therapeutic use by a patient. In one or more embodiments, the compositions described herein are physically stable with no visible phase separation when viewed with the naked eye and are chemically stable, as assessed by accumulation of the 4-epi degradant product below acceptable levels, for at least 6, 9, 12, 15, 18, 24, 27, 30, 33, or 36 months when stored at 5° C. before use. In one or more other embodiments, the compositions described herein are physically stable with no visible phase separation when viewed with the naked eye and are chemically stable, as assessed by accumulation of the 4-epi degradant product below acceptable levels, for at least 6, 9, 12, 15, 18, 24, 27, 30, 33, or 36 months when stored at 20° C. before use. In one or more embodiments, the compositions described herein are physically stable with no visible phase separation when viewed with the naked eye and are chemically stable, as assessed by accumulation of the 4-epi degradant product below acceptable levels, for at least 6, 9, 12, 15, 18, 24, 27, 30, 33, or 36 months when stored at 25° C. before use. In one or more embodiments, the compositions are physically stable with no visible phase separation when viewed with the naked eye and are chemically stable accumulating the 4-epi degradant product below acceptable levels for at least 1, 2, 3, 4, 5, or 6 months when stored at room temperature during use. In some embodiments, the tetracycline compositions described herein are substantially free of, essentially free of, or free of agents that may irritate, dehydrate, or defat skin, e.g., surfactants and/or ethanol. The compositions may therefore lead to better patient compliance.

Formulations disclosed herein may surprisingly form a physically stable homogenous entrapment of polar phase within an entrapment framework without any surface active agent. The formulations are thermally stable, can be applied easily to the skin and other surfaces, provide a pleasant and smooth skin feeling after application, do not cause skin irritation, and/or are readily absorbed into the skin.

Tetracycline antibiotics can have color and can stain clothing, potentially reducing patient compliance. To address these compliance factors, in some embodiments, the formulations described herein minimize or ameliorate color and/or cause little or no staining. Without being bound by any theory, this may be in part by the way in which the polar or hydrophilic phase is entrapped in the entrapment framework and in part because, when the tetracycline antibiotic is fully dissolved, there are no suspended particles remaining to effect staining.

In one or more embodiments, provided herein is a two phase homogenous carrier composition, the carrier composition comprising
i) an organic polar phase comprising at least one polyol; and
ii) an organic hydrophobic phase comprising a) at least one wax having a melting point above 37° C. and b) at least one hydrophobic solvent, at least one thickened silicone or both;
wherein the polar phase is entrapped within the hydrophobic phase; and wherein the composition is substantially free of a surfactant.

In one or more embodiments, provided herein is a two phase homogenous carrier composition, the carrier composition comprising
i) about 1% to about 70% by weight of the composition of an organic polar phase comprising at least one polyol; and
ii) an organic hydrophobic phase comprising a) about 5% to about 50% by weight of the composition at least one wax having a melting point of above 37° C. and b) about 10% to about 75% by weight of the composition of at least one hydrophobic liquid solvent;
wherein the polar phase is entrapped within the hydrophobic phase, wherein the ratio of wax to polyol is about or more than 0.5:1 and wherein the composition is substantially free of a surfactant.

In one or more embodiments, provided herein is a two phase homogenous carrier composition, the carrier composition comprising
i) about 1% to about 70% by weight of the composition of an organic polar phase comprising at least one polyol; and
ii) an organic hydrophobic phase comprising a) about 5% to about 50% by weight of the composition at least one wax having a melting point of above 37° C. b) about 10% to about 75% by weight of the composition of at least one hydrophobic liquid solvent and c) about 5% to about 40% by weight of the composition of at least one thickened silicone;
wherein the polar phase is entrapped within the hydrophobic phase, wherein the ratio of wax to polyol is about or more than 0.5:1 and wherein the composition is substantially free of a surfactant.

In one or more embodiments, provided herein is a two phase homogenous carrier composition, the carrier composition comprising
i) about 1% to about 70% by weight of the composition of an organic polar phase comprising at least one polyol; and
ii) an organic hydrophobic phase comprising a) about 5% to about 50% by weight of the composition at least one wax having a melting point of above 37° C. and b) about 15% to about 85% by weight of the composition of at least one thickened silicone;
wherein the polar phase is entrapped within the hydrophobic phase, wherein the ratio of wax to polyol is about or more than 0.5:1 and wherein the composition is substantially free of a surfactant.

In one or more embodiments, the composition is free or substantially free of water. In one or more embodiments, the composition is free of a surfactant.

In one or more embodiments, the wax comprises a plant wax, an animal wax, a petroleum derived wax, a vegetable wax, an albacer wax, an atlasenewax, a cardis wax, a ceramid, a beeswax, a BASF wax, a carnauba wax, a chinese wax, a cotton wax, a bayberry wax, a carnauba wax, a castor wax, a cuban palm wax, a duroxon wax, an esparto wax, a fat wax, a flax wax, a fischer-tropsch wax, a fir wax, a flexo wax, a flower wax, a glyco wax, a japan wax, a jojoba oil, a lanolin wax, a palm wax, a rice bran wax, a rice-oil wax, a shellac wax, a soy wax, an ucuhuba wax, a hydrogenated oil, a hydrogenated castor oil, a hydrogenated cottonseed oil, a hydrogenated jojoba oil, a mink wax, a mixture of saturated n- and isoalkanes, a montan wax, a naphthene, an ouricury wax, an oxazoline wax, an ozokerite, a paraffin wax, a paraffin 58-62° C. wax, a paraffin 57-60° C. wax, a paraffin 56-58 wax, a paraffin 52-54° C. wax, a paraffin 51-53° C. wax, a paraffin 46-48° C. wax, paraffin 42-44° C. wax, a microcrystalline wax, a polyethylene, a PEG-6 beeswax, a polyolefin, a polymekon wax, a retamo wax, a rezo wax, a sandy wax, a soy wax, a spent grain wax, a stearyl dimethicone, a sugarcane wax, a mineral wax, or a mixture of any two or more thereof.

In one or more embodiments, the wax comprises a paraffin wax 57° C.-60° C., a paraffin wax 51° C.-53° C., a paraffin wax 42° C.-44° C., a microcrystalline wax, a beeswax or a mixture thereof.

In one or more embodiments, the wax comprises a combination of at least two waxes. In one or more embodiments, the wax comprises a hydrocarbon-based wax. In one or more embodiments the hydrocarbon-based wax comprises a paraffin wax, a microcrystalline wax or a combination thereof. In one or more embodiments, the wax comprises a combination of at least two waxes, wherein at least one wax is a hydrocarbon-based wax. In one or more embodiments, the wax is substantially free, essentially free or free of non-hydrocarbon-based wax. In one or more embodiments, the wax is substantially free, essentially free or free of hydrogenated castor oil.

In one or more embodiments, the wax comprises a solid or semi-solid fatty alcohol or fatty acid, or a combination thereof.

In one or more embodiments, the wax does not comprises a solid or semi-solid fatty alcohol or fatty acid, or a combination thereof.

In one or more embodiments, the fatty alcohol is stearyl alcohol, cetyl alcohol, cetostearyl alcohol, myristyl alcohol, arachidyl alcohol, behenyl alcohol, or a mixture of any two or more thereof.

In one or more embodiments, the fatty acid is hexadecanoic acid, stearic acid, arachidic acid, behenic acid, octacosanoic acid, or a mixture of any two or more thereof.

In one or more embodiments, the fatty alcohol is having between 14 to 22 carbon atoms in its carbon chain.

In one or more embodiments, the fatty acid is having between 15 to 24 carbon atoms in its carbon chain.

In one or more embodiments, the wax or wax mixture has a melting point of at least about 42° C.

In one or more embodiments, the hydrophobic solvent is an oil selected from a mineral oil, a hydrocarbon oil, an ester oil, an ester of a dicarboxylic acid, a triglyceride oil, an oil of plant origin, an oil from animal origin, an unsaturated or polyunsaturated oil, a diglyceride, a PPG alkyl ether, an essential oil, a silicone oil, liquid paraffin, an isoparaffin, a polyalphaolefin, a polyolefin, polyisobutylene, a synthetic isoalkane, isohexadecane, isododecane, alkyl benzoate, alkyl octanoate, C12-C15 alkyl benzoate, C12-C15 alkyl octanoate, arachidyl behenate, arachidyl propionate, benzyl laurate, benzyl myristate, benzyl palmitate, bis (octyldodecyl stearoyl) dimer dilinoleate, butyl myristate, butyl stearate, cetearyl ethylhexanoate, cetearyl isononanoate, cetyl acetate, cetyl ethylhexanoate, cetyl lactate, cetyl myristate, cetyl octanoate, cetyl palmitate, cetyl ricinoleate, decyl oleate, diethyleneglycol diethylhexanoate, diethyleneglycol dioctanoate, diethyleneglycol diisononanoate, diethyleneglycol diisononanoate, diethylhexanoate, diethylhexyl adipate, diethylhexyl malate, diethylhexyl succinate, diisopropyl adipate, diisopropyl dimerate, diisopropyl sebacate, diisosteary dimer dilinoleate, diisostearyl fumerate, dioctyl malate, dioctyl sebacate, dodecyl oleate, ethylhexyl palmitate, ester derivatives of lanolic acid, ethylhexyl cocoate, ethylhexyl ethylhexanoate, ethylhexyl hydroxystarate, ethylhexyl isononanoate, ethylhexyl palmytate, ethylhexyl pelargonate, ethylhexyl stearate, hexadecyl stearate, hexyl laurate, isoamyl laurate, isocetyl behenate, isocetyl lanolate, isocetyl palmitate, isocetyl stearate, isocetyl salicylate, isocetyl stearate, isocetyl stearoyl stearate, isocetearyl octanoate, isodecyl ethylhexanoate, isodecyl isononanoate, isodecyl oleate, isononyl isononanoate, isodecyl oleate, isohexyl decanoate, isononyl octanoate, isopropyl isostearate, isopropyl lanolate, isopropyl laurate, isopropyl myristate, isopropyl palmitate, isopropyl stearate, isostearyl behenate, isosteary citrate, isostearyl erucate, isostearyl glycolate, isostearyl isononanoate, isostearyl isostearate, isostearyl lactate, isostearyl linoleate, isostearyl linolenate, isostearyl malate, isostearyl neopentanoate, isostearyl palmitate, isosteary salicylate, isosteary tartarate, isotridecyl isononanoate, isotridecyl isononanoate, lauryl lactate, myristyl lactate, myristyl myristate, myristyl neopentanoate, myristyl propionate, octyldodecyl myristate, neopentylglycol dicaprate, octyl dodecanol, octyl stearate, octyl palmitate, octyldodecyl behenate, octyldodecyl hydroxystearate, octyldodecyl myristate, octyldodecyl stearoyl stearate, oleyl erucate, oleyl lactate, oleyl oleate, propyl myristate, propylene glycol myristyl ether acetate, propylene glycol dicaprate, propylene glycol dicaprylate, maleated soybean oil, stearyl caprate, stearyl heptanoate, stearyl propionate, tocopheryl acetate, tocopheryl linoleate, glyceryl oleate, tridecyl ethylhexanoate, tridecyl isononanoate, triisocetyl citrate, an alexandria laurel tree oil, an avocado oil, an apricot stone oil, a barley oil, a borage seed oil, a calendula oil, a canelle nut tree oil, a canola oil, caprylic/capric triglycerides, a castor oil, a coconut oil, a corn oil, a cotton oil, a cottonseed oil, an evening primrose oil, a flaxseed oil, a groundnut oil, a hazelnut oil, glycereth triacetate, glycerol triheptanoate, glyceryl trioctanoate, glyceryl triundecanoate, a hempseed oil, a jojoba oil, a lucerne oil, a maize germ oil, a marrow oil, a millet oil, a neopentylglycol dicaprylate/dicaprate, an olive oil, a palm oil, a passionflower oil, pentaerythrityl tetrastearate, a poppy oil, propylene glycol ricinoleate, a rapeseed oil, a rye oil, a safflower oil, a sesame oil, a shea butter, a soya oil, a soybean oil, a sweet almond oil, a sunflower oil, a sysymbrium oil, a syzigium aromaticum oil, a tea tree oil, a walnut oil, wheat germ glycerides, a wheat germ oil, a PPG-2 butyl ether, a PPG-4 butyl ether, a PPG-5 butyl ether, a PPG-9 butyl ether, a PPG-12 butyl ether, a PPG-14 butyl ether, a PPG-15 butyl ether, a PPG-15 stearyl ether, a PPG-16 butyl ether, a PPG-17 butyl ether, a PPG-18 butyl ether, a PPG-20 butyl ether, a PPG-22 butyl ether, a PPG-24 butyl ether, a PPG-26 butyl ether, a PPG-30 butyl ether, a PPG-33 butyl ether, a PPG-40 butyl ether, a PPG-52 butyl ether, a PPG-53 butyl ether, a PPG-10 cetyl ether, a PPG-28 cetyl ether, a PPG-30 cetyl ether, a PPG-50 cetyl ether, a PPG-30 isocetyl ether, a PPG-4 lauryl ether, a PPG-7 lauryl ether, a PPG-2 methyl ether, a PPG-3 methyl ether, a PPG-3 myristyl ether, a PPG-4 myristyl ether, a PPG-10 oleyl ether, a PPG-20 oleyl ether, a PPG-23 oleyl ether, a PPG-30 oleyl ether, a PPG-37 oleyl ether, a PPG-40 butyl ether, a PPG-50 oleyl ether, a PPG-11 stearyl ether, a herring oil, a cod-liver oil, a salmon oil, a cyclomethicone, a dimethyl polysiloxane, a dimethicone, an epoxy-modified silicone oil, a fatty acid-modified silicone oil, a fluoro group-modified silicone oil, a methylphenylpolysiloxane, phenyl trimethicone, a polyether group-modified silicone oil, and a mixture of any two or more thereof.

In one or more embodiments the hydrophobic solvent is a plant oil, which is rich in triglycerides.

In one or more embodiments, the hydrophobic solvent is a hydrocarbon-based oil. In one or more embodiments, the hydrocarbon-based oil comprises petrolatum and/or mineral oil. In one or more embodiments, the hydrophobic solvent comprises a combination of at least two hydrophobic solvents. In one or more embodiments, the hydrophobic solvent comprises a combination of at least two hydrophobic solvents, wherein at least one hydrophobic solvent comprises a hydrocarbon-based hydrophobic solvent. In one or more embodiments, the hydrophobic solvent is substantially free, essentially free or free of non-hydrocarbon-based hydrophobic solvent. In one or more embodiments, the hydrophobic solvent is substantially free, essentially free or free of a vegetable oil, a soybean oil, isopropyl myristate, a cyclomethicone, a dimethicone or combinations of any two or more thereof.

In one or more embodiments, the hydrophobic solvent is a liquid fatty alcohol or a liquid fatty acid. In one or more embodiments, the liquid fatty alcohol is oleyl alcohol. In one or more embodiments, the liquid fatty acid is oleic acid. In one or more embodiments, the polyol comprises a triol. In one or more embodiments, the triol comprises glycerin, butane-1,2,3-triol, butane-1,2,4-triol, or hexane-1,2,6-triol. In one or more embodiments, the triol comprises glycerin. In one or more embodiments, the polyol comprises a diol. In one or more embodiments, the diol comprises propylene glycol, 1,2-propylene glycol, 1,3-propylene glycol, butanediol, 1,2-butanediol, 1,3-butanediol, 2,3-butanediol, 1,4-butanediol, butanediol, 1,3-butanediol, 1,4-butenediol, butynediol, pentanediol, pentane-1,2-diol, pentane-1,3-diol, pentane-1,4-diol, pentane-1,5-diol, pentane-2,3-diol, pentane-2,4-diol, hexanediol, hexane-1,6-diol, hexane-2,3-diol, hexane-2,56-diol, hexylene glycol, octanediol, 1,8-octanediol, neopentyl glycol, 2-methyl-1,3-propanediol, diethylene glycol, triethylene glycol, tetraethylene glycol, dipropylene glycol, or dibutylene glycol. In one or more embodiments, the composition further comprising a monohydric alcohol. In one or more embodiments, the monohydric alcohol is methanol, ethanol, n-propyl alcohol, isopropyl alcohol, n-butyl alcohol, t-butyl alcohol, 2-butanol, iso-butanol, pentanol, hexanol, or cyclohexanol. In one or more embodiments, the monohydric alcohol is isopropyl alcohol. In one or more embodiments, the composition comprises a diol and a triol, a monohydric alcohol and a diol, or a monohydric alcohol alcohol and a triol.

In one or more embodiments, the composition comprises:
up to about 40% by weight of the composition of a polyol;
about 5% to about 55% by weight of the composition of a wax; and
about 15% to about 75% by weight of the composition of a hydrophobic solvent.

In one or more embodiments, the composition comprises:
about or up to about 20% by weight of the composition of a polyol;
about 20% to about 42% by weight of the composition of a wax; and
about 40% to about 60% by weight of the composition of a hydrophobic solvent.

In one or more embodiments, the composition comprising:
up to about 25% by weight of the composition of a polyol;
about 35% to about 45% by weight of the composition of a wax; and
about 30% to about 45% by weight of the composition of a hydrophobic solvent.

In one or more embodiments, the composition comprising:
up to about 25% by weight of the composition of a polyol;
about 5% to about 15% by weight of the composition of a wax; and
about 60% to about 75% by weight of the composition of a hydrophobic solvent.

In one or more embodiments, the wax is a paraffin wax 57-60, the hydrophobic liquid substance is (i) a mineral oil or (ii) a mineral oil and a medium chain triglycerides oil, and the polyol is glycerin.

In one or more embodiments, the composition comprises:
about 15% to about 65% by weight of the composition of a polyol;
about 20% to about 45% by weight of the composition of a wax; and
about 10% to about 55% by weight of the composition of a hydrophobic liquid solvent.

In one or more embodiments, the composition comprising:
about 15% to about 45% by weight of the composition of a polyol;
about 20% to about 40% by weight of the composition of a wax; and
about 10% to about 30% by weight of the composition of a hydrophobic liquid solvent.

In one or more embodiments, the polyol comprises glycerin, propylene glycol, hexylene glycol, transcutol or a mixture thereof, the wax comprises stearyl alcohol, and wherein the hydrophobic solvent comprises oleic acid, oleyl alcohol, or a combination thereof.

In one or more embodiments, the composition comprising:
about 15% to about 25% by weight of the composition of a polyol;
about 25% to about 45% by weight of the composition of a wax; and
about 30% to about 55% by weight of the composition of a hydrophobic liquid solvent.

In one or more embodiments, the polyol is glycerin, the wax comprises a fatty alcohol, wherein the fatty alcohol comprises a stearyl alcohol and the hydrophobic liquid solvent is a mineral oil.

In one or more embodiments, the composition comprises:
up to about 40% by weight of the composition of a polyol;
about 5% to about 55% by weight of the composition of a wax;
about 15% to about 75% by weight of the composition of a hydrophobic solvent; and
about 5% to about 40% by weight of the composition of a thickened silicone.

In one or more embodiments, the composition comprises:
up to about 40% by weight of the composition of a polyol;
about 5% to about 55% by weight of the composition of a wax; and
about 5% to about 85% by weight of the composition of a thickened silicone.

In one or more embodiments, the composition comprises:
about or up to about 20% by weight of the composition of a polyol;
about 20% to about 42% by weight of the composition of a wax;
about 40% to about 60% by weight of the composition of a hydrophobic solvent; and
about 5% to about 40% by weight of the composition of a thickened silicone.

In one or more embodiments, the composition comprises:
about or up to about 20% by weight of the composition of a polyol;
about 5% to about 42% by weight of the composition of a wax; and
about 5% to about 85% by weight of the composition of a thickened silicone.

In one or more embodiments, the composition comprises:
up to about 25% by weight of the composition of a polyol;
about 60% to about 70% by weight of the composition of a hydrophobic solvent;
about 20% to about 45% by weight of the composition of a wax; and
about 5% to about 40% by weight of the composition of a thickened silicone.

In one or more embodiments, the composition comprising:
up to about 25% by weight of the composition of a polyol;
about 10% to about 45% by weight of the composition of a wax; and
about 5% to about 85% by weight of the composition of a thickened silicone.

In one or more embodiments, the composition comprises:
about 10% to about 65% by weight of the composition of a polyol;
about 20% to about 45% by weight of the composition of a wax;
about 10% to about 55% by weight of the composition of a hydrophobic liquid solvent; and
about 5% to about 40% by weight of the composition of a thickened silicone.

In one or more embodiments, the composition comprises:
about 10% to about 65% by weight of the composition of a polyol;
about 10% to about 45% by weight of the composition of a wax; and
about 5% to about 85% by weight of the composition of a thickened silicone.

In one or more embodiments, the composition comprises:
about 10% to about 45% by weight of the composition of a polyol;
about 20% to about 40% by weight of the composition of a wax;
about 10% to about 40% by weight of the composition of a hydrophobic liquid solvent; and
about 5% to about 40% by weight of the composition of a thickened silicone.

In one or more embodiments, the composition comprises:
about 10% to about 45% by weight of the composition of a polyol;
about 10% to about 40% by weight of the composition of a wax; and
about 5% to about 85% by weight of the composition of a thickened silicone.

In one or more embodiments, the composition comprises:
about 8% to about 25% by weight of the composition of a polyol;
about 20% to about 45% by weight of the composition of a wax;
about 30% to about 55% by weight of the composition of a hydrophobic liquid solvent; and
about 5% to about 40% by weight of the composition of a thickened silicone.

In one or more embodiments, the composition comprises:
about 10% to about 25% by weight of the composition of a polyol;
about 8% to about 45% by weight of the composition of a wax; and
about 45% to about 85% by weight of the composition of a thickened silicone.

In one or more embodiments the thickened silicone comprises a cyclopentasiloxane and dimethicone crosspolymer (ST-Elastomer 10), a stearoxytrimethylsilane and stearyl alcohol (Silky Wax 10), a dimethicone and polysilicone-11 (Gransil DMG5), a Cyclopentasiloxane (and) Polysilicone-11 (MGS-Elastomer 1100), a Cyclopentasiloxane (and) Petrolatum (and) Polysilicone-11 (MGS-Elastomer 1148P), a Polymethylsilsesquioxane (MGS powder 3300) or mixtures of any two or more thereof.

In one or more embodiments, the composition further comprises a propellant. In one or more embodiments, the gas propellant is a liquefied or a compressed gas propellant. In one or more embodiments, the gas propellant is present at a concentration of between about 4% to about 25% by weight of the composition. In some embodiments the ratio of propellant to the carrier including any active agent ranges from about 4:100 to 25:100 or from about 5:100 to 22:100 or from about 7:100 to 18:100. In one or more embodiments, when packaged in an aerosol container and pressurized with a propellant, the composition affords upon release from the container a foam that breaks upon application of shear force.

In one or more embodiments, the compositions further comprise at least one active agent.

In one or more embodiments, the active agent is suspended in the polar phase. In one or more embodiments, the active agent is fully or partially solubilized in the polar phase. In one or more embodiments, the active agent is suspended in the hydrophobic phase. In one or more embodiments, the active agent is fully or partially solubilized in the hydrophobic phase. In one or more embodiments, the active agent is solubilized in the hydrophobic phase and suspended in the polar phase. In one or more embodiments, the active agent is solubilized in the polar phase and suspended in hydrophobic phase. In one or more embodiments, the active agent is a tetracycline antibiotic. In one or more embodiments, the tetracycline antibiotics comprises a tetracycline, an oxytetracycline, a demeclocycline, a doxycycline, a lymecycline, a meclocycline, a methacycline, a minocycline, a rolitetracycline, a chlorotetracycline, or a tigecycline, and wherein the tetracycline antibiotic is present in a free base form, a hydrate form, a salt form, or a complex form.

In one or more embodiments, the composition further comprises a preservative or a stabilizer.

In one or more embodiments, provided herein is a method of treating or alleviating the symptoms of a dermatological, an ophthalmological, a gynecological, or a mucosal disorder, comprising: applying to a target area the composition described herein, wherein the disorder includes at least one etiological factor selected from the group consisting of an infection, an inflammation, oxidative stress, neurodegeneration, and apoptosis.

In one or more embodiments, the dermatological disorder is selected from the group consisting of an abscess, acne, acne conglobata, acne fulminans, acne vulgaris, acne scars, acute febrile neutrophilic dermatosis, acute lymphangitis, allergic contact dermatitis, alopecia, athlete’s foot, atopic dermatitis, bacterial skin infections, bullous pemphigoid, burn, calluses candidiasis, carbuncles, cellulitis, chemical burns, chicken pox, cholinergic urticaria, chronic effects of sunlight, comedones, corns, creeping eruption, cutaneous abscess, cutaneous myiasis, delusional parasitosis, dermatitis, dermatitis herpetiformis, dermatological inflammation, dermatophytoses, drug eruptions and reactions, dyshidrotic eczema, eczema, epidermoid cyst, epidermal necrolysis, exfoliative dermatitis, erythema multiforme, folliculitis, fungal nail infections, fungal skin infections, furuncles, gangrene, genital herpes, head lice, impetigo, inflammatory acne, ingrown nails, intertrigo, irritant contact dermatitis, ischemic necrosis, itching, jock itch, Kaposi’s sarcoma, molluscum contagiosum, MRSA, necrotizing subcutaneous infection, necrotizing fasciitis, necrotizing myositis, nodular papulopustular acne, non-inflammatory acne, nummular dermatitis, parapsoriasis paronychia, parasitic skin infections, pemphigus, photo-allergy, photo-damage, photo-irritation, photosensitivity, papules, pediculosis, perioral dermatitis, pimples, pityriasis rosea, pityriasis rosea, pityriasis rubra pilaris, poison ivy, post-operative or post-surgical skin conditions, pressure ulcers, pressure urticaria, pruritis, pseudofolliculitis barbae, psoriasis, pustules, rosacea, scabies, scarring, scleroderma, sebaceous cyst, seborrheic dermatitis, seborrheic keratosis, shingles, skin aging, skin rash, skin ulcers, staphylococcal scalded skin syndrome, sunburn, thermal burns, tinea corporis, tinea cruris, tinea pedis, tinea versicolor, toxic epidermal necrolysis, trauma or injury to the skin, varicella zoster virus, viral skin infections, wrinkles, and yeast skin infections.

In one or more embodiments, the gynecological or mucosal disorder is selected from the group consisting of a disorder of a body cavity or mucosal surface, a disorder of the nose, mouth, eye, ear, respiratory system, vagina, urethra, or rectum, chlamydia infection, gonorrhea infection, herpes, human papillomavirus (HPV), genital warts, bacterial vaginosis, candidiasis, molluscum contagiosum, nongonococcal urethritis (NGU), trichomoniasis, vulvodynia, yeast infection, pelvic inflammation, anal abscess/fistula, anal fissure, and hemorrhoids.

In one or more embodiments, the ophthalmological disorder is selected from the group consisting of an eye infection, an eye redness, eyelid problems, ophtahlmic allergy, blepharitis, corneal abrasion, corneal edema, corneal ulcer, conjunctivitis, contact lens complications, dry eye, eyelid cellulitis, glaucoma, macular degeneration, macular edema, ocular cicatricial pemphigoid, obstructed tear duct, ocular rosacea, optic neuritis, orbital cellulitis, recurrent corneal erosion, trachoma, and uveitis.

In one or more embodiments, the disorder is folliculitis, acne, rosacea or impetigo. In one or more embodiments, the ratio of hydrophobic phase to polar phase is between about 1.85:1 and about 1:1, or about 1.6:1, about 1.4:1, about 1.2:1, or about 1:1. In one or more embodiments, provided herein is a composition for use in the treatment of a disease or disorder. In one or more embodiments, provided herein is a use of the composition as herein disclosed in the manufacture of a medicament for treating a disease or disorder.

In one or more embodiments, provided herein is a homogenous carrier composition, the carrier composition comprising:
i) about 1% to about 50% by weight of the composition of an organic polar phase comprising at least one polyol; and
ii) an organic entrapment framework comprising a) about 5% to about 50% by weight of the composition at least one wax having a melting point of above 37° C. and b) about 10% to about 94% by weight of the composition of at least one hydrophobic liquid solvent;
wherein the polar phase is entrapped within the entrapment framework, and wherein the composition is substantially free of a surfactant.

In one or more embodiments, provided herein is a homogenous carrier composition, the carrier composition comprising:
i) about 1% to about 50% by weight of the composition of an organic polar phase comprising at least one polyol; and
ii) a hydrophobic phase comprising a) about 5% to about 50% by weight of the composition at least one wax having a melting point of above 37° C. b) about 10% to about 94% by weight of the composition of at least one hydrophobic liquid solvent and c) about 10% to about 35% of at least one thickened silicone;
wherein the polar phase is entrapped within the hydrophobic phase, and wherein the composition is substantially free of a surfactant.

In one or more embodiments, provided herein is a homogenous carrier composition, the carrier composition comprising:
i) about 1% to about 50% by weight of the composition of an organic polar phase comprising at least one polyol; and
ii) a hydrophobic phase comprising a) about 5% to about 50% by weight of the composition at least one wax having a melting point of above 37° C. and b) about 10% to about 85% of at least one thickened silicone;
wherein the polar phase is entrapped within the hydrophobic phase, and wherein the composition is substantially free of a surfactant.

In one or more embodiments, the carrier provided herein is a two-phase carrier composition, wherein the entrapment framework comprises or is the hydrophobic phase thereof. In some embodiments, the composition is essentially free of surface active agent. In some embodiments, the composition is free of surface active agents. In one or more embodiments, the composition is substantially waterless. In some embodiments, the composition is essentially waterless. In some embodiments, the composition is waterless.

In some embodiments, the polar phase is present in the composition at about 5% to about 60% by weight of the composition. In some embodiments, the polar phase is present in the composition betwen about 6% to about 55%, between about 7% to about 50%, between about 8% to about 48%, about 9% to about 47%, between about 10% to about 46%, between about 11% to about 45%, between about 12% to about 42%, between about 13% to about 40%, between about 14% to about 37%, between about 15% to about 35%, between about 16% to about 32%, about 17% to about 30%, between about 18% to about 28%, between about 19% to about 26%, or between about 20% to about 25%, by weight of the composition. In some embodiments, it is between about 10% to about 50%, between about 10% to about 40%, between about 10% to about 30%, about 12% to about 27%, between about 14% to about 46%, between about 16% to about 40%, between about 18% to about 35%, or between about 20% to about 45% by weight of the composition. In some embodiments, it is no more than about 40%, no more than about 35%, no more than about 30%, no more than about 25%, or no more than about 20% by weight of the composition. In some embodiments it is no less than about 7%, no less than about 10%, no less than about 13%, no less than about 15%, or no less than about 18% by weight of the composition. In some embodiments, it is about 40%, about 35%, about 30%, about 28%, about 26%, about 24%, about 22%, about 20%, about 18%, about 16%, about 14%, about 12%, about 10%, about 8%, about 7%, or about 5% by weight of the composition. In one or more embodiments, the polar phase is about 21% to about 19% by weight of the composition. In one or more embodiments, the polar phase comprises one or more polyols. In some embodiments the polyol is a triol. In some embodiments, the polar phase comprises a triol and a diol. In some embodiments, the polar phase comprises a triol and a monohydric alcohol. Suitable monohydric alcohols include propanol and transcutol. In some embodiments, the polar phase comprises a triol, a diol, and a monohydric alcohol. Suitable polyols include, without limitation, glycerin, hexylene glycol, and propylene glycol. In some embodiments, the polyol is glycerin. In other embodiments, the polyols comprise glycerin and propylene glycol. In yet other embodiments, the polyols comprise glycerin and hexylene glycol. In some embodiments, the composition comprises propylene glycol. In some embodiments, the monohydric alcohol comprises propanol. In some embodiments, the monohydric alcohol comprises transcutol. In some embodiments, the polar phase comprises propanol. In some embodiments, the polar phase comprises transcutol.

In some embodiments, the hydrophobic phase is present in the composition at about 25% to about 90% by weight of the composition. In some embodiments, it is between about 25% to about 85% or between about 25% to about 80% by weight of the composition. In some embodiments, it is between about 30% to about 90%, between about 33% to about 87%, between about 35% to about 85%, between about 38% to about 82%, or between about 40% to about 80% by weight of the composition. In some embodiments, it is at least about 30%, at least about 40%, at least about 50%, at least about 60%, at least about 65%, or at least about 70% by weight of the composition. In one or more embodiments, the hydrophobic phase is about 70%, about 72%, about 74%, about 76%, about 78%, about 80%, about 82%, about 84%, about 86%, about 88%, about 90%, or about 92% by weight of the composition.

In some embodiments, the hydrophobic phase comprises a wax or a mixture of waxes. In some embodiments, the hydrophobic phase comprises a wax or a mixture of waxes in combination with a hydrophobic substance. In some embodiments, the wax comprises a paraffin wax. In some embodiments, the wax comprises a solid fatty acid. In yet another embodiment, the wax comprises a solid fatty alcohol. In some embodiments the wax comprises a beeswax. In some embodiments the wax comprises a microcrystalline wax.

In some embodiments, the hydrophobic substance comprises an oil. In some embodiments, the hydrophobic substance comprises a semi-solid oil (at room temperature). In some embodiments, the hydrophobic substance comprises a hydrophobic emollient. In some embodiments, the hydrophobic substance comprises liquid oil. In some embodiments, the hydrophobic substance comprises a hydrophobic liquid solvent. In an embodiment, the oil is a mineral oil. In a further exemplary embodiment, the hydrophobic substance is a liquid fatty acid. In yet another embodiment, the hydrophobic substance is a liquid fatty alcohol. In some embodiments, the hydrophobic substance comprises a hydrophobic solvent, a wax and a thickened silicone, or a wax and a thickened silicone.

In one embodiment, the formulation is semi solid. In one embodiment, the formulation is an oleaginous composition. In one embodiment, the composition is a non-emulsion or a non-classic dispersion. In yet another embodiment, the formulation is a gel. In yet another embodiment, the formulation is an ointment. In another embodiment, the formulation is in the form of a foam or mouse. In yet other embodiments, the formulation is a foamable composition comprising the carrier, a tetracycline antibiotic, foaming additives as appropriate and a propellant. In some embodiments, the formulations comprises solid or semi-solid materials such as high melting point waxes (e.g., having a melting point from about 65° C. to about 125° C.), fatty acid esters, fatty acid amides, fatty acids, fatty alcohols, and polymeric materials in amounts less than those required to formulate a composition with the consistency of a stick or lipstick. A skilled artisan would understand that such amounts may vary depending on the materials used. Generally, it is appropriate to look at the total amount of solid and semi-solid material present in context of the total composition, not merely the individual components, although some components such as polymeric agents can create very thick or gelled materials in relatively low amounts. In one or more embodiments, the total amount of such solid or semi-solid materials other than wax is not more than about 10%, not more than about, 12%, not more than about 14%, not more than about 16%, not more than about 18%, not more than about 20%, not more than about 22%, not more than about 24%, or not more than about 25% by weight of the composition. Non-limiting examples of such solid or semi-solid materials include high melting point waxes (e.g., having a melting point from about 65° C. to about 125° C.), fatty acid esters, fatty acid amides, fatty acids, fatty alcohols, and polymeric materials. Compositions that are hard are considered disadvantageous and unsuited for topical delivery of active agents to skin or mucosa. Compositions having the consistency of a stick or lipstick may require the application of pressure or friction to transfer composition to skin or mucosa, which is inappropriate where patients have sore or broken, damaged, or inflamed skin or mucosa. In yet another embodiment, the composition is substantially free of solid emulsifying or silica-based particles. In yet another embodiment, the composition is essentially free of emulsifying or silica-based solid particles. In yet another embodiment, the composition is free of emulsifying or silica-based solid particles. All the aforesaid forms are formulated to provide stability of the tetracycline and may be applied topically onto skin or mucosa surfaces. Additionally, the novel carriers described herein can be used with other active agents to deliver them topically onto skin or mucosa surfaces. For example, active agents that are soluble in polar solvents can be selected for use in the polar phase. Likewise, active agents that are soluble in hydrophobic solvents can be selected for use in the hydrophobic phase. Not only can the carriers described herein be used for and with unstable active agents, they can also be used for and with stable active agents. Non- limiting examples of active agents that can be used in the carriers in addition to tetracycline antibiotics or in place of tetracycline antibiotics are provided herein.

### Definitions

All % values are provided on a weight (w/w) basis.

Various carriers and compositions or formulations are described herein. They are often described for use in a method. A reference to or example of a carrier, composition or formulation for use in one method does not in any way limit the carrier, composition or formulation for use just in that method, but it can be for use in any other method or embodiment described herein. The carriers, compositions or formulations described herein are in one or more embodiments provided as carriers, compositions or formulations and are in one or more embodiments provided as a product even where they are described only in relation to their use in a method.

As used herein, the term “about” has its usual meaning in the context of pharmaceutical and cosmetic formulations to allow for reasonable variations in amounts that can achieve the same effect. By the term “about” herein it is meant as indicated above and also that a figure or range of figures can vary in an embodiment plus or minus up to 20%. For example, if an amount of “about 2” is provided, then the amount can reflect a variation of from 1.6 up to 2.4. In further embodiments, it can describe a variation of plus or minus 10%, in which case “about 1” can reflect a variation of from 0.9 up to 1.1. As will be appreciated by one skilled in the art in cases where “about X” will lead to a figure of above 100%, the term in one or more embodiments can be read as reflecting up to 100% by weight less the total of the minimum amount of the other ingredients. Likewise, it will be appreciated by one skilled in the art to the extent X is reduced from that upper level the amounts of the other ingredients are increased appropriately. As will be appreciated by one of skill in the art, there is some reasonable flexibility in formulating compositions such that where one or more ingredients are varied, successful formulations can still be made even if an amount falls slightly outside the range. Therefore, to allow for this possibility, amounts are qualified by about. In one or more other embodiments, the figures can be read without the term “about.”

As used herein, the terms “composition(s)” and “formulation(s)” can be interchangeable depending on the context in which they are used as would be appreciated by a person skilled in the art.

As used herein, the terms “liquisoft complex” refers to a complex arrangement that forms a two-phase homogenous composition, one phase being a polar liquid and the other a hydrophobic fluid solid or semi solid without the presence of surfactants to bridge and interlink the hydrophobic and polar phases. In one or more embodiments, the liquisoft complex comprises a polar phase that is entrapped or covered by a hydrophobic phase, also called entrapment framework. In one or more embodiments, the liquisoft complex comprises a hydrophobic phase that is entrapped or covered by a polar phase. In one or more embodiments, the liquisoft complex comprises a polar phase that is entrapped or covered by a hydrophobic phase, wherein said hydrophobic phase is further entrapped or covered by a polar phase. In one embodiment, the liquisoft complex comprises a multi-layered structure comprising complex layers or aliquots of polar phase and hydrophobic phase, wherein each layer entraps or covers the layer beneath it. In the multi-layer framework on one level some oil wax covers some polar solvent, and on another level some other aliquot of polar solvent covers part or all that oil wax and on another level some or all of that aliquot of polar solvent is covered with oil wax and so on.

As used herein the term “entrapment framework” refers to a semi-solid medium capable of homogenously entrapping a dispersed phase (e.g. a wax or an oil diluted wax capable of entrapping a polar phase within the wax) thereby producing a “pseudo-dispersion” or “non-classic-dispersion” without the use of customary surfactants or the use of emulsifying or silica-based solid particles. In some embodiments, the entrapment framework refers to a hydrophobic semi-solid medium or hydrophobic phase (e.g., a hydrophobic solvent and a wax, a hydrophobic solvent, a wax and a thickened silicone, or a wax and a thickened silicone) capable of entrapping a hydrophilic or polar phase substance (e.g., a polyol(s)). The admixture of polar and hydrophobic phases may appear as one homogenous phase when viewed by the naked eye because the polar phase is effectively evenly distributed throughout the hydrophobic phase. In one or more embodiments, the entrapment framework can serve to provide a generally, substantially, or essentially homogenous composition. As used herein the term “pseudo-dispersion” or “non-classic-dispersion” refers to a homogenous entrapment of a first substance within a second substance without the use of surfactants or emulsifying or silica-based solid particles at the interface of the two substances. In some embodiments, pseudo-dispersion within the context herein can refer to entrapment or distribution of hydrophilic or polar phase substance(s) (e.g., a polyol(s)) within hydrophobic phase substance(s) (e.g., a hydrophobic solvent and a wax, a hydrophobic solvent, a wax and a thickened silicone, or a wax and a thickened silicone). In contrast, “classic dispersions” rely on the surface active properties and/or wetting properties of components located at the interface between the dispersed and the continuous phase to stabilize the dispersion whereas “pseudo-dispersions” or “non-classic-dispersion” do not require these surface active agents or wetting agents to stabilize the dispersed phase within the entrapment framework.

In some embodiments, the homogenous entrapment is “generally homogenous,” meaning the concentration of the entrapped substance is substantially same throughout the composition demonstrating variations of about ± 10% or less, ± 8% or less, or ± 5% or less, ± 3% or less, ± 2% or less, or ± 1% or less, or ± 0.8% or less, or ± 0.6% or less, in the concentration of the entrapped substance between different samplings. The homogeneity of the entrapped polar phase can be assessed visually by the naked eye or under a light microscope, or by sampling a portion of the composition and determining the concentration of the entrapped polar phase. For instance, to assess homogeneity, each sample is collected from a different portion of a single composition, with each sample taken at the same or different time points. At least two or more samples are obtained from a composition, and the concentration of the entrapped substance of each sample is measured. The concentration variation among the samples reflect the homogeneity of the entire composition.

In some embodiments, the homogenous entrapment is “substantially homogenous,” meaning the concentration of the entrapped substance is substantially same throughout the composition demonstrating variations of about ± 0.5% or less, ± 0.4% or less, or ± 0.3% or less, ± 0.2% or less ± 0.1% or less, or ± 0.08% or less in the concentration of the entrapped substance between different samplings.

In some embodiments, the homogenous entrapment is “essentially homogenous” such that the concentration of the entrapped substance is essentially same throughout the composition demonstrating variations of about ± 0.05% or less, ± 0.01% or less, or ± 0.001% or less between samplings.

In some embodiments, the homogenous entrapment is “completely homogenous” so that the concentration of the entrapped substance is effectively the same throughout the composition. In some embodiments, the entrapped phase may present any form or structure, such as droplets, micelles, entrapment of particles, or tubules at various sizes.

As used herein the term “polar phase” refers to a fraction of the composition comprising a polar solvent. In an embodiment, the polar phase comprises a polar liquid solvent. In a further embodiment, the polar phase comprises a hydrophilic solvent. In yet another embodiment, the polar phase comprises a polyol or a mixture of polyols, or one or more polyols with other polar or hydrophilic solvent.

As used herein, the term a “hydrophilic solvent” refers to a solvent that has a solubility in distilled water at ambient temperature of more than about 1 gm per 100 mL, or more than about 0.5 gm per 100 mL, or even more than about 0.1 gm per 100 mL. The hydrophilic solvent remains a liquid at ambient temperature.

As used herein the terms “hydrocarbon-based oil” or “hydrocarbon-based hydrophobic solvent” refer to an oil or a hydrophobic solvent, which is free, essentially free or substantially free of polar groups. For e.g. mineral oil and petrolatum.

As used herein the term “hydrocarbon-based wax” refers to a wax which is free, essentially free or substantially free of polar groups for e.g. paraffin wax and microcrystalline wax

As used herein the terms “non-hydrocarbon-based oil” and “non-hydrocarbon-based wax” refer to an oil or a wax, respectively, which contains polar groups e.g., soybean oil or hydrogenated castor oil.

As used herein the term “hydrophobic phase” is used interchangeably with the term “apolar phase” and refers to a fraction of the composition that contains hydrophobic solvents, either solid, semi-solid or liquids, or a mixture thereof. In some embodiments, the hydrophobic phase includes at least one wax. In some embodiments, the hydrophobic phase includes at least one hydrophobic liquid solvent. In some embodiments, the hydrophobic phase includes a thickened silicone. In other embodiments, the hydrophobic phase includes at least one wax and at least one hydrophobic liquid solvent. In other embodiments, the hydrophobic phase includes at least one wax and at least one thickened silicone. In other embodiments, the hydrophobic phase includes at least one wax, at least one hydrophobic liquid solvent and at least one thickened silicone. The term “viscosity-modifying agent” or “thickening agent” in the context of the present disclosure is an agent, which modulates the viscosity or thickness of a formulation. The viscosity-modifying agent, when provided with a polar phase solvent and a hydrophobic solvent further advantageously facilitates homogenous entrapment of the polar solvent within the composition and affords a physically stable composition devoid from any phase separation for prolonged time. According to the present disclosure, the viscosity-modifying agent can be selected from a semi-solid or a solid wax component (e.g., a paraffin wax, a fatty alcohol, and a fatty acid). In some embodiments, it is a fatty alcohol. In some embodiments, it is a fatty acid. In some embodiments, it is a paraffin wax. In some embodiments, if a composition described herein comprises a fatty alcohol, a fatty acid, or both, the composition does not form an oil gel or oil diluted wax gel. For instance, the composition may be semi solid at rest and liquid on application of mechanical or shear forces such as light force generally used to spread a soft petrolatum. In the context of the present disclosure, the fatty alcohols, fatty acids, and waxes are selected to be compatible with tetracycline antibiotics, and in particular, with a doxycycline or with a minocycline.

As used herein the term “solubilized” is used interchangeably with the term “dissolved” and refers to an active pharmaceutical agent (API) or a fraction thereof that is dissolved in a volume fraction of a liquid substance (i.e., solvent). The composition is therefore substantially free of, essentially free of, or free of any emulsifying or silica-based solid particles derived from the active agent. In some embodiments, the API is solubilized in the polar solvent (e.g., polyol) and provided in a topical formulation as herein disclosed in a solubilized form.

The term “fully solubilized” refers to the entire API provided in the formulation being solubilized in the polar solvent and provided in a topical formulation as herein disclosed in a fully solubilized form.

The term “partially solubilized” or “partially dissolved” has the meaning of an API wherein a fraction thereof is solubilized and another fraction is suspended in the composition. In some embodiments, an API is partially solubilized and partially suspended in the herein disclosed compositions.

In one or more embodiments, the ratio of suspended to solubilized API in the formulations of the disclosure is between about 1:10 to about 10:1, between about 1:8 to about 8:1, between about 1:6 to about 6:1, between about 1:4 to about 4:1, between about 1:2 to about 2:1, or any intermediate value. In some embodiments, the ratio of solubilized vs. suspended API in the formulations of the disclosure is about 1:1, about 1:2, about 1:3, about 1:4, about 1:5, about 1:6, about 1:7, about 1:8, about 1:9, or about 1:10, or any intermediate value. In some embodiments, the ratio of suspended vs. solubilized API in the formulations of the disclosure is about 1:1, about 1:2, about 1:3, about 1:4, about 1:5, about 1:6, about 1:7, about 1:8, about 1:9, or about 1:10, or any intermediate value.

The terms “room temperature” and “ambient temperature” are used interchangeably and refer to USP Controlled room temperature defined as 20° C. to 25° C. In some embodiments, it is 20° C. In some embodiments, it is 21° C. In some embodiments, it is 22° C. In some embodiments, it is 23° C. In some embodiments, it is 24° C. In some embodiments, it is 25° C.

As used herein, “semi-solid waxy substance” or “solid waxy substance” includes any hydrophobic material which may be solid or semi-solid at room temperature or at body temperature. The term encompasses fatty alcohols, fatty acids and waxes which are solid or semi-solid at room temperature.

As used herein the terms “solid”, “semi-solid” and “liquid” refer to the physical state of a substance at room temperature.

The term “body temperature” as used herein, means normal body temperature of about 36° C. to about 38° C. In some embodiments, it is about 36° C. In some embodiment, it is about 37° C. In some embodiments, it is about 38° C.

The terms “surfactant,” “surface active agent,” and “emulsifier,” in the context used herein, refer to stand alone compounds used to reduce surface tension between two substances or phases, and which are also capable of stabilizing an emulsion of water and oil. Reduction of surface tension can be significant in foam technology in relation to the ability to create small stable bubbles. “Surfactant” and “emulsifier,” as used herein, do not include compounds which do not function effectively as standalone compounds for reducing surface tension between two substances or phases and which are not capable of stabilizing an emulsion of water and oil. For example, a surfactant or emulsifier as provided herein does not include fatty acids, does not include fatty alcohols, and does not include propoxylated lanolin oil derivatives. In the context of the present disclosure, fatty acids and fatty alcohols are defined as foam adjuvants or wax. Similarly, propoxylated lanolin oil derivatives in the context herein are defined as emollients.

The terms “standard surfactant,” “customary surfactant” or “stand alone surfactant” refer to customary non-ionic, ionic, anionic, cationic, zwitterionic, amphoteric and amphiphilic surfactants. Many standard surfactants are derivatives of fatty alcohols or fatty acids, such as ethers or esters formed from such fatty alcohols or fatty acids with hydrophilic moieties, such as polyethylene glycol (PEG). However, a native (non-derivatized) fatty alcohol or fatty acid, as well as waxes are not regarded as a standard surfactant in this application.

The term “co-surfactant” as used herein means a molecule which on its own is not able to form and stabilize satisfactorily an oil-in-water emulsion but aids a customary surfactant to improve emulsion stability. For instance, cetyl alcohol may be considered a co-surfactant as it is a waxy hydrophobic substance that can be emulsified with water using a surfactant. In one or more embodiments the compositions are substantially free, essentially free or free of co-surfactant.

As used herein, the term “de-minimis” amount of an ingredient as provided throughout the specification is intended to mean that the composition comprises an amount having no functional effect. For instance, when a composition comprising two phases comprises a “de-minimis” amount of a surface active agent, the amount of the surface active agent is insufficient to emulsify the two phases. In one or more embodiments, the composition comprises a de-minimis amount of one or more of a surfactant, a polymeric agent, a gelling agent, a film forming agent, a fatty acid, a fatty alcohol, a diester, a divalent metal cation, a cyclodextrin, a dehydrating agent, an antioxidant, a sulphite, a monohydric aliphatic alcohol, an aromatic alcohol, a molecule with SiOH groups, or water. In some embodiments, the “de-minimis” amount of an ingredient encompasses a trace amount of the ingredient, i.e., less than about 10 ppmw (parts per million by weight).

It should be noted that the term “substantially free of” an ingredient as provided throughout the specification is intended to mean that the composition comprises less than about 0.5% by weight, less than about 0.4% by weight, less than about 0.3% by weight, less than about 0.2% by weight, or less than about 0.1% by weight, of an ingredient unless specifically indicated otherwise. In some embodiments, a composition “substantially free of” an ingredient comprises less than 0.5% by weight, less than 0.4% by weight, less than 0.3% by weight, less than 0.2% by weight, or less than 0.1% by weight of the ingredient.

As used herein, the term “essentially free of” an ingredient as provided throughout the specification is intended to mean that the composition comprises less than about 0.05% by weight, less than about 0.01% by weight, or less than about 0.001% by weight of the ingredient, unless specifically indicated otherwise. In some embodiments, a composition “essentially free of” an ingredient comprises less than 0.05% by weight, less than 0.01% by weight, or less than 0.001% by weight of the ingredient.

As used herein, the term “free of” an ingredient as provided throughout the specification is intended to mean that the composition does not comprise the ingredient or comprises a trace amount of the ingredient, unless specifically indicated otherwise.

As used herein, the terms “substantially surfactant free” or “substantially surface active agent free” means the composition contains about or less than 0.5% by weight; about or less than 0.4% by weight; about or less than 0.3% by weight; about or less than 0.2% by weight; or about or less than 0.1% by weight of a surfactant.

As used herein, “effectively free of surfactant” or “effectively free of surface active agent” indicates less than about 0.08% by weight, less than about 0.05% by weight, less than about 0.01 % by weight, or less than about 0.001 % by weight of a surfactant.

The terms “surfactant-free” or “emulsifier-free” or “non-surfactant” refer to compositions which comprise no or trace levels of surfactants, emulsifiers, or surface active agents.

As used herein, the term “low water content” has the meaning of water content below about 5%, below about 4%, below about 3%, below about 2.5%, below about 2%, below about 1.5%, or below about 1% by weight of water. In some embodiments, the term has the meaning of below 5%, below 4%, below 3%, below 2.5%, below 2%, below 1.5%, or below 1% by weight of water.

As used herein, the terms “substantially waterless,” “substantially water free,” and “substantially non-aqueous,” all of which are used interchangeably herein, mean the composition contains about or less than 1.0%; about or less than 0.75%; about or less than 0.5% by weight; about or less than 0.4% by weight; about or less than 0.3% by weight; about or less than 0.2% by weight; or about or less than 0.1% by weight of water.

In one or more embodiments, the composition is “essentially water-free,” indicating that the composition comprises less than about 0.05% by weight, less than about 0.01% by weight, or less than about 0.001% by weight of water. In some embodiments, the term has the meaning of less than 0.05% by weight, less than 0.01% by weight, or less than 0.001% by weight of water.

The terms “waterless,” “water-free,” and “non-aqueous,” all of which are used interchangeably herein, refer to a composition that contains no free or unassociated or absorbed water, or negligible amounts thereof.

A composition may absorb water from the atmosphere. Also, when purchased from manufacturers, ingredients used to formulate a composition may contain associated or unfree water complexed with the other ingredients by covalent, electrostatic, or other types of interactions, or water may be absorbed from the atmosphere to interact with these ingredients after manufacture. For instance, a composition described herein may contain up to 1% by weight of water from these sources even though no free water was added to the composition. Thus, unless otherwise indicated, the amount of water encompassed by the terms “low water content,” “substantially free of,” “essentially free of,” or “free of” water added as a separate ingredient to the composition does not necessarily exclude the presence of further water in the final product from other sources, such as water absorbed by the composition from the atmosphere and/or water associated with one or more of the other ingredients used in the composition.

The term “physically stable” as used herein refers to a composition or formulation that exhibits no phase separation and or crystallization visible to the naked eye. In some embodiments, the herein disclosed compositions are physically stable for at least about 1 month, for at least about 2 months, for at least about 3 months, for at least about 4 months, for at least about 6 months, for at least about 12 months, for at least about 18 months, or for at least about 24 months at room temperature. In some embodiments, the herein disclosed compositions are physically stable for at least about 1 month, for at least about 2 months, for at least about 3 months, for at least about 4 months, for at least about 6 months, for at least about 12 months, for at least about 18 months, or for at least about 24 months, incubated at 25° C. In some embodiments, at least about 1 month, for at least about 2 months, for at least about 3 months, for at least about 4 months, for at least about 6 months, for at least about 12 months, for at least about 18 months, or for at least about 24 months, incubated at 25° C. In some embodiments, the compositions disclosed herein are physically stable for at least about 1 month, for at least about 2 months, for at least about 3 months, for at least about 4 months, for at least about 6 months, or for at least about 12 months, incubated at 30° C. In some embodiments, the herein disclosed composition is physically stable for at least about 1 month, for at least about 2 months, for at least about 3 months, for at least about 4 months, or for at least about 6 months, incubated at 40° C.

In some embodiments, the herein disclosed composition is physically stable after centrifugation at 1,000 rpm for at least about 1 minute, for at least about 2 minutes, for at least about 3 minutes, for at least about 4 minutes, for at least about 5 minutes, for at least about 10 minutes, for at least about 20 minutes, for at least about 30 minutes, or for at least about 60 minutes. In some embodiments, the herein disclosed composition is physically stable after centrifugation at 3,000 rpm for at least about 1 minute, for at least about 2 minutes, for at least about 3 minutes, for at least about 4 minutes, for at least about 5 minutes, for at least about 10 minutes, for at least about 20 minutes, for at least about 30 minutes, or for at least about 60 minutes. In some embodiments, the herein disclosed composition is physically stable after centrifugation at 10,000 rpm for at least about 1 minute, for at least about 2 minutes, for at least about 3 minutes, for at least about 4 minutes, for at least about 5 minutes, for at least about 10 minutes, for at least about 20 minutes, for at least about 30 minutes, or for at least about 60 minutes.

The term “non-grainy,” “non-grainy texture,” or “non-grainy feeling” refers to a composition that does not include aggregates, or is essentially free or is substantially free of solid aggregates (e.g., a solid substance, such as a wax or a solid fatty alcohol/acid). The grainy texture or lack of it is determined according to feel following discernment of the composition upon the skin or other body surface. In some embodiments, the herein disclosed compositions are smooth presenting no or essentially no grainy feeling.

By the term “phase separation,” it is meant that the admixture of polar and hydrophobic phases exhibits two visible phases as viewed by the naked eye.

As described herein, it is hypothesized that the polar phase is essentially or substantially homogeneously entrapped within the apolar or hydrophobic phase. According to some embodiments, the polar phase may form structures such as micelles, droplets, tubules, or the alike within the apolar phase. In some embodiments, a composition described herein appears without magnification as a single phase because the polar phase is homogenously or generally homogenously distributed throughout the hydrophobic phase.

By the term “two phase homogenous composition,” it is meant to refer to a composition initially comprising two visually separate phases, i.e., a polar phase and a hydrophobic phase, wherein following admixture of polar and hydrophobic phases, the resulting formulation exhibits a “substantially homogeneous entrapment.”

The terms “non-emulsion” and “non-classic dispersion” refer to a composition as herein disclosed that includes a polar phase and a hydrophobic phase, wherein the composition exhibits a substantially or essentially single phase in a composition free, substantially free, or essentially free of a surfactant. In contrast, the terms “emulsion” or “classic-emulsion” or “classic dispersion” generally refer to compositions that require surfactants to stabilize one phase dispersed in the other phase. The terms surfactant, surface active agent and emulsifier are used interchangeably herein.

By the term “a non-classical ointment” it is meant to refer to the herein disclosed compositions being in the form of an ointment that include a polar phase and a hydrophobic phase. It is to be noted that “classical ointments” include primarily hydrophobic substances (such as oils and waxes). Ointments are typically viscous, semi-solid preparations, most commonly a greasy, thick oil. The current “non-classical ointment” includes homogenous polar phase and a hydrophobic phase composition with a low water content, or that are substantially, essentially or waterless.

By “a low entropy composition” it is meant to refer to a composition in which the polar and hydrophobic phases once present in an essentially uniform distribution or entrapment of the polar phase in the hydrophobic phase, are able to remain so for a substantial or long period of time with no or little energy input.

The term “unstable” as used herein, means a compound, e.g., an active agent, which is degraded within less than a day, and in some cases, in less than an hour, upon exposure to air, light, skin, or water or a pharmaceutical excipient, under ambient conditions.

The terms “stable” or “chemically stable” as used herein, means a compound, e.g., an active agent, which is degraded upon exposure to air, light, skin, or water or pharmaceutical excipient, under ambient conditions, in an acceptable breakdown levels or below, or in comparable lower levels.

In some embodiments, by way of example the rate of appearance of a 4-epi breakdown product of a tetracycline antibiotic is less than about 1% per month at 25° C. For example, less than about 0.9% per month, less than about 0.8% per month, less than about 0.7% per month, less than about 0.6% per month, less than about 0.5% per month, less than about 0.4% per month, less than about 0.3%, less than about 0.2%, less than about 0.1%, or less than about 0.05% per month of the tetracycline antibiotic breaks down and produces a 4-epi breakdown product when incubated at 25° C. In some embodiments, the rate of appearance of an 4-epi breakdown product is less than about 2% per month, when incubated at 40° C. For example, less than about 1.8% per month, less than about 1.6% per month, less than about 1.4% per month, less than about 1.2% per month, less than about 1% per month, or less than about 0.8% per month of the tetracycline antibiotic breaks down and produces a 4-epi breakdown product, when incubated at 40° C.

In some embodiments, the predicted amount of 4-epi breakdown product at 25° C. following 6 months is about 1.5% or less, about 1.4% or less, about 1.3% or less, about 1.2% or less, about 1.1% or less, about 1% or less, about 0.9% or less, about 0.8% or less, about 0.7% or less, about 0.6% or less, about 0.5% or less, about 0.4% or less, about 0.3% or less, about 0.2% or less, or about 0.1% or less. In some embodiments, the predicted amount of 4-epi breakdown product at 25° C. following 24 months is about 6% or less, about 5% or less, about 4.5% or less, about 4% or less, about 3.5% or less, about 3% or less, about 2.5% or less, about 2% or less, about 1.5% or less, about 1% or less, or about 0.5% or less.

In some embodiments, the predicted amount of 4-epi breakdown product at 30° C. following 6 months is about 5% or less, about 4.5% or less, about 4.2% or less, about 4% or less, about 4% or less, about 3.9% or less, about 3.8% or less, about 3.7% or less, about 3.6% or less, or about 3.5% or less, 3.4% or less, about 3.3% or less, about 3.2% or less, about 3.1% or less, about 3% or less, about 2.9% or less, about 2.8% or less, about 2.7% or less, about 2.6% or less, or about 2.5% or less, about 2.4% or less, about 2.3% or less, about 2.2% or less, about 2.1% or less, about 2% or less, about 1.9% or less, about 1.8% or less, about 1.7% or less, about 1.6% or less, about 1.5% or less, about 1.4% or less, about 1.3% or less, about 1.2% or less, about 1.1% or less, or about 1% or less.

In some embodiments, the predicted amount of 4-epi breakdown product at 40° C. following 6 months is about 6% or less, about 5% or less, about 4.5% or less, about 4.2% or less, about 4% or less, about 4% or less, about 3.9% or less, about 3.8% or less, about 3.7% or less, about 3.6% or less, or about 3.5% or less, about 3% or less, about 2.5% or less, about 2% or less, about 1.5% or less, about 1% or less, or about 0.5% or less.

In various embodiments, tetracycline antibiotics degradation can be detected by measuring epimerization at the C-4 position (amine), resulting in the appearance of epimer-4 (referred as “epi-4” or “4-epi”), epimerization at the C-6 position (amine), resulting with epimer-6 (referred as “epi-6” or “6-epi”), appearance or appearance of other impurities (designated herein as total impurities, “TI”). The aforementioned degradation products can undergo additional epimerization to thereby form alternative degradation products. Tetracycline degradation may be assessed by measuring the degradants by liquid chromatography (epi-4 and/or epi-6 and/or total impurities) and/or monitoring the tetracycline content by liquid chromatography (referred to herein for doxycycline as a “DOX assay”) in the composition, and/or monitoring the color change over time of the tetracycline within the tested compositions.

As used herein, the term “TI value” means the level of total impurities other than epi-4 and epi-6, assessed by UHPLC (ultra-high performance liquid chromatography).

As used herein, the term “epi-4” or “4-epi” refers to tetracycline degradant epimer-4.

As used herein, the term “epi-6” or “6-epi” refers to tetracycline degradant epimer-6.

In some embodiments, the term “an acceptable breakdown level” of tetracycline means that less than about 6% of the tetracycline breaks down as 4-epi following 6 months of incubation at 40° C. or less than 6% of the tetracycline breaks down as 4-epi following 2 years of incubation at 25° C. Other embodiments are described herein.

As used herein, the term “cosmeceutical agent” refers to a cosmetic agent that has one or more medicinal, therapeutic, and/or drug-like benefits. Non-limiting examples include alpha hydroxy acids (AHAs), beta hydroxy acids (BHAs) and polyhydroxy acids (PHAs), vitamins such as vitamin A, vitamin B, vitamin B3, vitamin C, vitamin D, vitamin D3, vitamin E, vitamin derivatives, lipoic acid, salicylic acid, keratolytic agents, peeling agents, depigmenting (or bleaching) agents such as hydroquinone and kojic acid, botanical and marine extracts, UV filters, UV absorbers, lactic acid, retinol, retinoid and nicotinamide. In some embodiments, a cosmeceutical agent may be a pharmaceutical agent that is used at low doses to provide one or more benefits to skin and mucosa. In one or more embodiments, a cosmeceutical agent can manipulate and/or modulate the biological function of the skin, e.g., improve appearance of the skin, such as skin tone, texture, clarity and/or wrinkles by delivering nutrients essential for healthy skin.

As used herein, the term “an active agent stabilizer” has the meaning of an agent that contributes, affords, mediates or promotes stability of the active agent (such as an unstable tetracycline antibiotic, e.g., doxycycline) within the composition for prolonged time. In some embodiments, compositions comprising an active agent stabilizer present reduced measurement of at least one degradant of the active agent as compared to corresponding compositions without the stabilizer. In some embodiments, compositions comprising an active agent stabilizer present a higher measurement of the active agent as compared to corresponding compositions without the stabilizer. In some embodiments, compositions comprising an active agent stabilizer present a lower color score evaluation as compared to corresponding compositions without the stabilizer.

The term “breakable” refers to a property of a gel or foam wherein the gel or foam is stable upon dispensing from a container, yet breaks and spreads easily upon application of shear or mechanical force, which can be mild, such as a simple rub.

As used herein, the term “preventing” refers to avoiding or delaying the onset of a disorder or condition from occurring in a subject that has not yet been diagnosed as having the disorder or condition, but who may be susceptible to it.

As used herein, the term “treatment” refers to inhibiting the disorder or condition, arresting its development, relieving the disorder or condition, causing regression of the disorder or condition or reversing the progression of the disorder or condition, or relieving or reducing one or more symptoms of the disorder or condition.

It should be noted that the term “a method of preventing, treating a disease or a disorder” as provided throughout the specification is interchangeable with the term “use of the composition as a medicament for preventing or treating a disease.” It should be noted that the term “disease” is used interchangeably with the term “disorder.”

The term “hydrophobic solvent” refers to a substance that has a low or no solubility in water. In one embodiment, 100 to 1000 parts of water are needed to dissolve or render miscible 1 part of the hydrophobic solvent. In another embodiment, 1000 to 10,000 parts of water are needed to dissolve or render miscible 1 part of the hydrophobic solvent. In yet another embodiment, more than 10,000 parts of water are needed to dissolve or render miscible 1 part of the hydrophobic solvent.

The term “polyol” as used herein refers to an organic substance that contains at least two hydroxyl groups in its molecular structure.

According to some embodiments, the tetracycline antibiotics is in a solubilized form. According to some embodiments, the composition is free, or essentially free, or substantially free of a surface active agent. According to some embodiments, the composition is free, or essentially free, or substantially free of water. According to some embodiments, the composition is free, or essentially free, or substantially free of water of a dehydrating agent. According to some embodiments, the composition is free, or essentially free, or substantially free of a monohydric aliphatic alcohol. According to some embodiments, the monohydric aliphatic alcohol is selected from the group consisting of ethanol, isopropanol, propyl alcohol, tert-butyl alcohol, and any combination thereof. According to some embodiments, the dehydrating agent is ethyl acetate, acetic anhydride or ethanol. According to some embodiments, the composition is free or essentially free, or substantially free of a polymeric agent or water gelling agent or film forming agent.

In one or more embodiments there is provided a liquisoft complex composition comprising:
a. a polar phase;
b. a hydrophobic phase;
wherein the polar phase is pseudo-dispersed within the hydrophobic phase without a surfactant to interlink and stabilize the hydrophobic and polar phases.

In some embodiments the polar phase of the liquisoft composition is substantially homogeneously mixed with the hydrophobic phase. In some embodiments the hydrophobic phase of the liquisoft composition is a flowable solid or semi solid. In some embodiments the hydrophobic phase of the liquisoft composition forms an entrapment framework and the polar phase is substantially entrapped within the entrapment framework. In some embodiments the polar phase comprises a polyol. In some embodiments the polyol comprises a triol, in some the polyol comprises a triol and a diol, and in some the polyol comprises a triol and a monohydric alcohol. In some embodiments the triol comprises glycerol. In some embodiments the diol comprises a glycol. In some embodiments the monohydric alcohol comprises a short chain alcohol with a C1 to C6 carbon chain. In some embodiments the glycol comprises propylene or hexylene glycol.

In some embodiments the liquisoft composition further comprises a polymeric agent. In one or more embodiments the entrapment framework of the liquisoft composition comprises a wax or combinations of two or more waxes having a melting point above 25° C. and a hydrophobic solvent. In some embodiments the wax or combinations thereof have a melting point above 37° C. and in others a melting point above 42° C. In one or more embodiments the wax comprises one or more of a paraffin wax, a microcrystalline wax, a beeswax, or a hydrogenated oil. In some embodiments, the hydrophobic phase of the liquisoft composition includes a solid or semi-solid fatty alcohol or fatty acid with a carbon chain of less than C22.

In some embodiments the hydrophobic phase of the liquisoft composition comprises an oil, in some a petrolatum, and in some both. In some embodiments the oil is liquid at 25° C. and comprises one or more of a mineral oil, a plant oil, an oil rich in triglycerides, a medium chain triglyceride oil, or a soybean oil. In some embodiments the hydrophobic phase comprises a silicone oil. In some embodiments, the silicone oil comprises a cyclomethicone, a dimethicone or both. In some embodiments it comprises a thickened silicone in addition to the wax or to the wax and oil. In one or more embodiments the thickened silicone comprises one or more of a cyclopentasiloxane and dimethicone crosspolymer (ST-Elastomer 10), a stearoxytrimethylsilane and stearyl alcohol (Silky Wax 10), a dimethicone and polysilicone-11 (Gransil DMG5), a cyclopentasiloxane (and) polysilicone-11 (MGS-Elastomer 1100), a cyclopentasiloxane (and) petrolatum (and) polysilicone-11 (MGS-Elastomer 1148P), or a polymethylsilsesquioxane (MGS powder 3300). In some embodiments the entrapment framework comprises a combination of two or more thickened silicones.

In some embodiments the thickened silicone is about 5% to about 40% by weight of the composition. In some embodiments the ratio of wax to polyol is about or more than 0.5:1 or more than 1:1. In some embodiments, the ratio between the wax to the polyol is about or more than about 2:1. In some embodiments the ratio of the wax to the polyol is about or more than about 3:2. In some embodiments, the ratio of wax to polar solvent is between about 0.5:1 and about 8:1. In some embodiments the ratio of wax to polar solvent is between about 1:1 and about 4:1. In some embodiments the ratio of wax to polar solvent is between about 3:2 and about 4:1. In some embodiments the ratio of hydrophobic phase to polar phase is between about 1:1 and about 9:2. In some embodiments the ratio of hydrophobic phase to polar phase is between about 3:2 and about 4:1.

In some embodiments, the entrapment framework is complex and comprises multi-layers or levels. In one or more embodiments, the polar phase of the liquisoft composition comprises about 5% to about 45% by weight of the composition; and the hydrophobic phase comprises about 15% to about 95% by weight of the composition of at least one wax. In one or more embodiments the polar phase of the liquisoft composition comprises about 25% to about 45% by weight of the composition; and the hydrophobic phase comprises about 30% to about 75% by weight of the composition of at least one wax. In one or more embodiments the polar phase of the liquisoft composition comprises about 30% to about 40% by weight of the composition; and the hydrophobic phase comprises about 40% to about 70% by weight of the composition of at least one wax. In one or more embodiments the polar phase of the liquisoft composition comprises about 1% to about 45% by weight of the composition; and the hydrophobic phase comprises a) about 1% to about 50% by weight of the composition of at least one wax; and b) about 10% to about 80% by weight of the composition of at least one hydrophobic liquid solvent. In some embodiments the polar phase comprises about 5% to about 40% by weight of the composition; and the hydrophobic phase comprises a) about 5% to about 45% by weight of the composition of at least one wax; and b) about 10% to about 75% by weight of the composition of at least one hydrophobic liquid solvent. In some embodiments the polar phase comprises about 8% to about 35% by weight of the composition; and the hydrophobic phase comprising a) about 8% to about 43% by weight of the composition of at least one wax; and b) about 10% to about 70% by weight of the composition of at least one hydrophobic liquid solvent.

In some embodiments the polar phase comprises about 10% to about 30% by weight of the composition; and the hydrophobic phase comprises a) about 10% to about 42% by weight of the composition of at least one wax; and b) about 15% to about 65% by weight of the composition of at least one hydrophobic liquid solvent. In some embodiments the polar phase comprises about 15% to about 25% by weight of the composition; and the hydrophobic phase comprising a) about 35% to about 45% by weight of the composition of at least one wax; and b) about 20% to about 50% by weight of the composition of at least one hydrophobic liquid solvent. In some embodiments the polar phase comprises about 15% to about 35% by weight of the composition; and the hydrophobic phase comprises a) about 15% to about 43% by weight of the composition of at least one wax; and b) about 10% to about 60% by weight of the composition of at least one hydrophobic liquid solvent.

In some embodiments the polar phase comprises about 15% to about 41% by weight of the composition; and the hydrophobic phase comprises a) about 20% to about 45% by weight of the composition of at least one wax; and b) about 10% to about 40% by weight of the composition of at least one hydrophobic liquid solvent. In some embodiments the polar phase comprises about 18% to about 45% by weight of the composition; and the hydrophobic phase comprises a) about 20% to about 44% by weight of the composition of at least one wax; and b) about 10% to about 50% by weight of the composition of at least one hydrophobic liquid solvent. In some embodiments the polar phase comprises up to about 35% by weight of the composition of a polyol; and the hydrophobic phase comprises a) about 5% to about 45% by weight of the composition of at least one wax; and b) about 30% to about 75% by weight of the composition of at least one hydrophobic liquid solvent. In some embodiments the polar phase comprises up to about 28% by weight of the composition of a polyol; and the hydrophobic phase comprises a) about 15% to about 45% by weight of the composition of at least one wax; and b) about 30% to about 65% by weight of the composition of at least one hydrophobic liquid solvent.

In some embodiments the polar phase comprises up to about 25% by weight of the composition of a polyol; and the hydrophobic phase comprises a) about 5% to about 25% by weight of the composition of at least one wax; and b) about 40% to about 65% by weight of the composition of at least one hydrophobic liquid solvent. In some embodiments the polar phase comprises up to about 20% by weight of the composition of a polyol; and the hydrophobic phase comprises a) about 18% to about 42% by weight of the composition of at least one wax; and b) about 35% to about 63% by weight of the composition of at least one hydrophobic liquid solvent. In some embodiments the polar phase comprises about 15% to about 25% by weight of the composition of a polyol; and the hydrophobic phase comprises a) about 18% to about 42% by weight of the composition of at least one wax; and b) about 35% to about 63% by weight of the composition of at least one hydrophobic liquid solvent. In some embodiments the polar phase comprises about 15% to about 25% by weight of the composition of a polyol; and the hydrophobic phase comprises a) about 25% to about 45% by weight of the composition of at least one wax; and b) about 30% to about 55% by weight of the composition of at least one hydrophobic liquid solvent. In some embodiments, the composition is gel-like, and in some embodiments, it is ointment-like.

In one or more additional embodiments the liquisoft composition further comprises a propellant. In some embodiments the propellant is a liquefied or a compressed gas propellant. In some embodiments the propellant is present at a concentration of between about 4% to about 25% of the composition. In some embodiments when packaged in an aerosol container and pressurized with the propellant, the composition affords upon release from the container a foam. In some embodiments the foam breaks upon application of shear force. In some embodiments the foam is quick break and breaks without application of a shear force.

In one or more embodiments the composition is a pharmaceutical composition and includes one or more active pharmaceutical agents. In one or more embodiments, the composition is a cosmeceutical composition and includes one or more cosmeceutical agents. In some embodiments the active pharmaceutical agent or cosmeceutical agent is dissolved in one of the phases. In some embodiments there are two active pharmaceutical agents, or two active cosmeceutical agents, or one active pharmaceutical agent, and one active cosmeceutical agent with one dissolved in the polar phase, and one in the hydrophobic phase. In one or more embodiments the active pharmaceutical agent is dissolved in the polar phase. In one or more embodiments the cosmeceutical agent is dissolved in the polar phase.

In one or more embodiments the liquisoft composition is substantially free of water other than water that is associated with the active pharmaceutical agent or associated with the cosmeceutical agent. In one or more embodiments the concentration of active pharmaceutical agent in the polar phase or that of the cosmeceutical agent in the polar phase is higher than in the composition.

In one or more embodiments the active pharmaceutical agent/cosmeceutical agent is stable in the liquisoft composition. In some embodiments, its stability when concentrated in the polar solvent in the composition is improved compared to its stability in the polar solvent alone. In some embodiments the active pharmaceutical agent is a tetracycline antibiotic. In some embodiments it comprises a doxycycline and in some the doxycycline is doxycycline hyclate.

In one or more embodiments the active pharmaceutical agent is present in an effective pharmaceutical concentration for topical application to the skin or mucosa to treat a skin disorder involving a bacterial infection and or inflammation. In one or more embodiments the disorder is acne or rosacea. In one or more embodiments the polar solvent enhances penetration of the active pharmaceutical agent into the skin or mucosa.

In some embodiments the concentration of active pharmaceutical agent or in the liquisoft composition is about 0.5% to about 5% by weight of the composition. In some embodiments the concentration of active pharmaceutical agent in the polar phase is about 1.5% to about 25%. In some embodiments the concentration of active pharmaceutical agent in the liquisoft composition is about 1.5% to about 2.5% by weight of the composition. In some embodiments the concentration of active pharmaceutical agent in the polar phase is about 4.5% to about 13%. In some embodiments the concentration of cosmeceutical agent or in the liquisoft composition is about 0.1% to about 5% by weight of the composition. In some embodiments the concentration of cosmeceutical agent in the polar phase is about 0.3% to about 25%. In some embodiments the concentration of cosmeceutical agent in the liquisoft composition is about 0.5% to about 2.5% by weight of the composition. In some embodiments, the concentration of cosmeceutical agent in the polar phase is about 1.5% to about 13%.

In one or more embodiments, the liquisoft composition is substantially free or essentially free of metal cations and or is substantially free or essentially free of a sulfite antioxidant and or is substantially free or essentially free of a dehydrating agent comprising ethyl acetate, acetic anhydride or ethanol. In some embodiments it is free of one or more of a sulfite antioxidant, and a dehydrating agent comprising ethyl acetate, acetic anhydride or ethanol.

In one or more embodiments the liquisoft composition retains above about 90% of the active pharmaceutical agent initially present in the composition after storage of the composition at 25° C. for at least three months. In one or more embodiments the liquisoft composition retains above about 90% of the active pharmaceutical agent initially present in the composition after storage at 5° C. for at least three months. In one or more embodiments the active pharmaceutical agent of the liquisoft composition is a doxycycline and less than about 0.9% breaks down to 4-epi after storage at 25° C. for at least three months. In one or more embodiments the active pharmaceutical agent the liquisoft composition is a doxycycline and less than about 0.9% breaks down to 4-epi after storage at 5° C. for at least three months. In one or more embodiments the liquisoft composition retains above about 90% of the active pharmaceutical agent initially present in the composition after storage at 25° C. for at least six months.

In one or more embodiments the liquisoft composition retains above about 90% of the active pharmaceutical agent initially present in the composition after storage at 5° C. for at least six months. In one or more embodiments the active pharmaceutical agent of the liquisoft composition is a doxycycline and less than about 1.8% breaks down to 4-epi after storage at 25° C. for at least six months. In one or more embodiments active pharmaceutical agent of the liquisoft composition is a doxycycline and less than about 1.8% breaks down to 4-epi after storage at 5° C. for at least six months. In one or more embodiments the liquisoft composition retains above about 90% of the cosmeceutical agent initially present in the composition after storage at 25° C. for at least six months. In one or more embodiments the liquisoft composition retains above about 90% of the cosmeceutical agent initially present in the composition after storage at 5° C. for at least six months.

The liquisoft composition may be provided and combined in any one or more of the preceding embodiments. In any one or more of the preceding embodiments, the liquisoft composition is administered to the skin or mucosa to treat a disorder. In any one or more of the preceding embodiments, there is provided a liquisoft composition, for use in the treatment of a dermatological, an ophthalmological, a gynecological, or a mucosal disease or disorder.

In one or more embodiments there is further provided a novel method of increasing stability of an unstable active pharmaceutical agent, comprising: adding an unstable active agent in to either a polar phase or a hydrophobic phase in the liquisoft composition of any of the preceding embodiments at a concentration in which the active pharmaceutical agent is soluble but at a concentration that is less than that in which it recrystallizes at 25° C.; dissolving the agent in that phase with warming; and mixing the phase including the dissolved agent with the other phase at a temperature at which the hydrophobic phase is liquid and cooling with mixing to form a substantially homogenous composition; wherein the active pharmaceutical agent is at a concentration in the phase in which is dissolved of at least twice than that in the composition; and wherein its solubility in the phase in which it is dissolved in the composition is higher than that in the same solvent alone. In one or more embodiments its stability in the phase in which it is dissolved in the composition is higher than that in the same solvent alone. In one or more embodiments the higher its concentration in the phase in which it is dissolved in the composition the higher its stability. In some embodiments the concentration in the phase in which the active pharmaceutical agent is dissolved is at least four times than that in the composition, and in some embodiments it is at least five times than that in the composition. In some embodiments the active pharmaceutical agent is a doxycycline and it is dissolved in the polar phase, and in an embodiment the doxycycline is doxycycline hyclate.

In one or more embodiments there is provided a use of the composition of any one of the preceding embodiments in the manufacture of a medicament for treating a dermatological, an ophthalmological, a gynecological, or a mucosal disease or disorder. In one or more embodiments there is provided a method of treating or alleviating the symptoms of a dermatological, an ophthalmological, a gynecological, or a mucosal disorder, comprising: applying to a target area the liquisoft composition of any of the preceding embodiments, wherein the disorder includes at least one etiological factor selected from the group consisting of an infection, an inflammation, oxidative stress, neurodegeneration, and apoptosis. In one or more embodiments the disorder is an inflammation of skin, mucosa or eye, and or the disorder is an infection of skin, mucosa or eye. In some embodiments the disorder is an acne and/or a rosacea. In some embodiments, the disorder is an acne. In some embodiments, the disorder is a rosacea.

In one or more embodiments there is provided a liquisoft complex composition comprising:
a. a polar phase comprising a dissolved doxycycline; and
b. a flowable or semi-solid hydrophobic phase;
wherein the polar phase is pseudo-dispersed homogeneously within the hydrophobic phase without a surfactant to interlink and stabilize the hydrophobic and polar phases. In some embodiments the hydrophobic phase of the liquisoft composition forms an entrapment framework and the polar phase is substantially entrapped within the entrapment framework, wherein the entrapment framework comprises a wax or combinations of two or more waxes having a melting point above 37° C. and a hydrophobic solvent. In some embodiments the wax comprises one or more of a paraffin wax, a microcrystalline wax, a beeswax, and a hydrogenated oil. In some embodiments the hydrophobic solvent comprises an oil that is liquid at 25° C. In some embodiments the polar phase comprises a polyol, wherein the polyol comprises a triol, a triol and a diol, or a triol and a monohydric alcohol. In some embodiments the oil comprises one or more of a mineral oil, a plant oil, an oil rich in triglycerides, a medium chain triglyceride oil, and a soybean oil. In some embodiments the triol comprises glycerol. In some embodiments the diol, if present, comprises a glycol.

In some embodiments the monohydric alcohol, if present, comprises a short chain alcohol with a C1 to C6 carbon chain. In some embodiments the ratio of wax to polyol is about or more than 0.5:1. In some embodiments the ratio of hydrophobic phase to polar phase is about 1:1: to about 5:1. In some embodiments the hydrophobic phase of the liquisoft composition further comprises a petrolatum, a silicone oil, a thickened silicone or mixtures of any two or more thereof.

In some embodiments the polar phase of the liquisoft composition comprises up to about 35% by weight of the composition of a polyol; and the hydrophobic phase comprises a) about 5% to about 45% by weight of the composition of at least one wax; and b) about 30% to about 75% by weight of the composition of at least one hydrophobic liquid solvent. In some embodiments the polar phase of the liquisoft composition comprises about 15% to about 25% by weight of the composition of a polyol; and the hydrophobic phase comprises a) about 18% to about 42% by weight of the composition of at least one wax; and b) about 35% to about 63% by weight of the composition of at least one hydrophobic liquid solvent.

In some embodiments the liquisoft composition is substantially free of water other than water that is associated with the active pharmaceutical agent. In some embodiments the liquisoft composition is a gel or ointment like and when mixed with a liquefied or a compressed gas propellant in a canister can form a foam upon release, wherein the ratio of composition to propellant is between about 100:4 to about 100:25. In some embodiments the concentration of doxycycline in the polar phase is higher than in the composition.

In some embodiments the stability of doxycycline in the polar solvent in the composition is improved compared to its stability in the polar solvent alone. In some embodiments when stored at 25° C. for three months, the liquisoft composition retains above about 90% of the doxycycline initially present in the composition and less than about 0.9% breaks down to 4-epi; or wherein when stored at 5° C. for six months, the composition retains above about 90% of the doxycycline initially present in the composition and less than about 1.8% breaks down to 4-epi.

The liquisoft composition may be provided and combined in any one or more of the preceding embodiments. In any one or more of the preceding embodiments, doxycycline is doxycycline hyclate and is present in an effective pharmaceutical concentration for topical application to the skin or mucosa to treat a skin disorder involving a bacterial infection and or inflammation. In some embodiments the concentration of doxycycline hyclate is about 0.5% to about 3.5% in the composition and is about 1.4% to about 10% in the polar phase. In some embodiments the disorder comprises an acne or a rosacea.

In any one or more of the preceding embodiments, there is provided a liquisoft composition, for use in the treatment of a dermatological, an ophthalmological, a gynecological, or a mucosal disease or disorder, or there is provided its use in the manufacture of a medicament having activity for the treatment thereof.

In any one or more of the preceding embodiments, there is provided a method of stabilizing or increasing stability of a doxycycline, comprising: dissolving the doxycycline in a polar phase at a concentration in which the doxycycline is soluble but below the concentration in which it recrystallizes at 25° C.; and dispersing the polar phase comprising the dissolved doxycycline in a hydrophobic phase to form a substantially homogenous composition; wherein concentration of doxycycline in the polar phase is at least twice than that in the composition; and wherein the stability of doxycycline in the composition is higher compared to the stability of the same concentration of doxycycline in an equivalent polar phase that is not dispersed in a hydrophobic phase. In some embodiments the dispersion is a pseudo-dispersion and the polar phase is pseudo dispersed in the hydrophobic phase.

In some embodiments there is provided a method of stabilizing or increasing stability of a doxycycline, comprising: adding the doxycycline to the polar phase in a liquisoft composition at a concentration in which the doxycycline is soluble but below the concentration in which it recrystalizes at 25° C.; dissolving the doxycycline in the polar phase; mixing the polar phase comprising the dissolved doxycycline with the hydrophobic phase at a temperature at which the hydrophobic phase is liquid; and cooling with mixing to form a substantially homogenous composition; wherein the concentration of doxycycline in the polar phase is at least twice than that in the composition; and wherein its stability in the composition is higher than the stability of the same concentration of doxycycline in an equivalent polar phase that is not pseudo-dispersed within the hydrophobic phase. In some embodiments the doxycycline is doxycycline hyclate.

In some embodiments there is provided a method of treating or alleviating the symptoms of a dermatological, an ophthalmological, a gynecological, or a mucosal disorder, comprising: applying to a target area a liquisoft composition, wherein the disorder includes at least one etiological factor selected from the group consisting of an infection, an inflammation, oxidative stress, neurodegeneration, and apoptosis. In any one or more of the preceding embodiments, the disorder is an inflammation of skin, mucosa or eye and or wherein the disorder is an infection of skin, mucosa or eye, wherein the polar phase of the liquisoft composition comprises about 15% to about 25% by weight of the composition; and wherein the hydrophobic phase comprises a) about 35% to about 45% by weight of the composition of at least one wax; and b) about 20% to about 50% by weight of the composition of at least one hydrophobic liquid solvent. In some embodiments, the disorder is an acne or a rosacea. In some embodiments, the disorder is an acne. In some embodiments, the disorder is a rosacea.

### Properties of the Disclosed Compositions

In various embodiments, a composition comprising a polar phase and a hydrophobic phase disclosed herein exhibits one or more favorable properties such as (1) efficient entrapment of the polar phase within the hydrophobic phase, (2) homogeneous dispersion of the entrapped polar phase throughout the hydrophobic phase, (3) increased solubility for the active ingredient(s), e.g., tetracycline antibiotics, in the polar phase, (4) reduced and/or delayed degradation of the active ingredient, (5) delivery of active agent into the skin or mucosa with little or no systemic penetration and suitable for application to skin or mucosa. In some embodiments, the formulation provides good properties for all of the listed parameters. In some embodiments, the formulation provides for physical and chemical stability of the active agent in the formulation (e.g., an unstable agent such as doxycycline) for at least about 1, 2, 3, 4, 5, 6, or more months.

In certain embodiments, a composition disclosed herein comprises a polar phase entrapped in a hydrophobic phase. The entrapment of the polar phase can be assessed e.g. by visual inspection with the naked eye for absence of phase separation and/or by adding a dye that is only soluble in the polar phase and examination under a light microscope. For instance, when the polar phase remains entrapped in the hydrophobic phase, no visible phase separation may be seen by naked eyes and therefore the composition may appear homogenous. In some embodiments, the composition is substantially free of, essentially free of, or free of surfactants (including surface acting solid particles) that are traditionally used to generate an oil-in-water or water-in-oil emulsion and yet, the polar phase remains entrapped in the hydrophobic phase. In some embodiments, the polar phase remains substantially entrapped in the hydrophobic phase for a prolonged period, e.g., 1 week, 1 month, 6 months, 1 year, or more, when incubated at about 5° C., In some embodiments at about 25° C.; in some embodiments at about 30° C.; in some embodiments at about 35° C.; or in some embodiments at about 40° C. For instance, the polar phase may remain entrapped in the hydrophobic phase for at least about 1 week, for at least about 2 weeks, for at least about 3 weeks, for at least about 1 month, for at least about 2 months, for at least about 3 months, for at least about 4 months, for at least about 6 months, for at least about 12 months, for at least about 18 months, or for at least about 24 months, when incubated at about 5° C., 25° C., 30° C., 35° C., or 40° C.

In some embodiments, the entrapped polar phase is homogenously dispersed throughout the hydrophobic phase. The homogeneity of the entrapped polar phase can be assessed visually, by the naked eye or under a light microscope or by sampling a portion of the composition and determining the concentration of the entrapped polar phase. As defined above, when the entrapment is “generally homogenous,” the concentration of the entrapped substance is generally the same throughout the composition, demonstrating variations of about ± 10% or less, ± 8% or less, or ± 5% or less, ± 3% or less ± 2% or less, or ± 1% or less, or ± 0.8% or less, or ± 0.6% or less, in the concentration of the entrapped substance between different samplings from the composition. When, the entrapment is “substantially homogenous,” the concentration of the entrapped substance is substantially the same within the composition demonstrating variations of about ± 0.5% or less, ± 0.4% or less, ± 0.3% or less, ± 0.2% or less, ± 0.1% or less, or ± 0.08% or less in the concentration of the entrapped substance between different samplings. When, the entrapment is “essentially homogenous” the concentration of the entrapped substance is essentially the same within the composition demonstrating variations of about ± 0.05% or less, ± 0.01% or less, or ± 0.001% or less between samplings. When the entrapment is “completely homogenous,” the concentration of the entrapped polar phase is to the naked eye effectively the same throughout the composition.

In some embodiments, the polar phase of a composition described herein provides increased solubility for the active agent, e.g., a tetracycline antibiotic, as compared to a composition having a polar phase alone. For instance, when a polar phase is entrapped in a hydrophobic phase as described herein, an active agent, e.g., tetracycline antibiotic, may be solubilized in the polar phase at a significantly higher concentration than the saturating concentration of the active agent in a polar phase not entrapped in a hydrophobic phase. In some embodiments, the polar phase described herein is capable of solubilizing an active agent, e.g., a tetracycline antibiotic, at a concentration at least about 2%, 5%, 10%, 20%, 30%, 40%, 50%, 60%, 70%, 80%, 90%, 100% or higher than a saturating concentration of the active agent in a polar phase alone.

In some embodiments, the composition described herein slows down the degradation of an active agent in the composition. In some embodiments, the active agent is a tetracycline antibiotic. The degradation of tetracycline antibiotic may be assessed by assessing the amount of tetracycline degradant epimer-4 (“epi-4”) in the composition. The rate of degradation is calculated by measuring the amount of epi-4 in the composition, plotted against time. The degradation of tetracycline antibiotic may also be assessed by monitoring color change of the composition over time. Color change may be quantified by comparing the composition’s original color value (e.g., light yellow) and the value assessed later. A color scoring may use a scale ranging from 0 to 3 (0= light/pale yellow, 1= yellow, 2= dark yellow to light brown, and 3= brown to black). In some embodiments, a composition described herein slows down the rate of degradation of tetracycline antibiotic by at least about 5%, 10%, 20%, 30%, 40%, 50%, 60%, 70%, 80%, 90%, or 100% as compared to the rate of degradation measured in a composition comprising a polar phase alone.

In some embodiments, a composition capable of delivering an active agent to skin and/or mucosa does not systematically penetrate into the blood stream in significant amounts or amounts that can generate unwanted systemic side effects. The penetration level of the compositions described herein may be assessed, e.g., using Franz diffusion cells in vitro. For example, a composition comprising an active agent is applied onto a portion of porcine ear skin for 24 hours using Franz diffusion cells. Following incubation, the skin piece is stripped from the cell and the amount of the active agent present in each sublayer of the skin piece (e.g., stratum comeum-1 external layer, stratum corneum-2 internal layer, and viable skin containing the epidermis and dermis) is analyzed. The amount of the active agent is also measured in the receiver compartment of the cell. If the amount of the active agent present in the receiver compartment is relatively lower than that present in the skin pieces, it may indicate that the composition is capable of delivering the active agent into the skin, but not through the skin and therefore has a lower risk of systemic penetration. In some embodiments, when a composition comprising an active agent as described herein is assessed by Franz diffusion cells, the amount of the active agent present in the skin piece is at least at least about 10%, 20%, 30%, 40%, 50%, 60%, 70%, 80%, 90%, or 100% higher as compared to the amount of the active agent present in the receiver compartment of the cell.

In some embodiments, a composition described herein possesses one or more functional properties described above. The functional properties may be achieved by adjusting various factors, e.g., types of polar or hydrophobic ingredients used in the composition, the amount of each ingredient, and/or the ratio among the ingredients.

In some embodiments, the polar phase of the composition comprises a polar solvent. In some embodiments, the polar solvent is a polyol. In some embodiments, the polyol is glycerin. Glycerin in the composition may be present at about 5% to about 40% by weight of the composition, at about 15% to about 30% by weight of the composition, about 15% to about 25% by weight of the composition, about 15% to about 20% by weight of the composition, or any percentage in between. In some embodiments, glycerin is present in the composition at about 5%, about 10%, about 15%, about 20%, about 25%, or about 30% by weight of the composition. In some embodiments, glycerin is present in the composition about 20% by weight of the composition. In one or more embodiments glycerol is a prime component to solubilize an active pharmaceutical ingredient such as doxycycline. In some embodiments glycerol is present in an effective amount so that all the active pharmaceutical ingredient, such as doxycycline is solubilized. In some embodiments the amount solubilized is directly proportional to the amount of glycerol. In some embodiment, the polar phase further comprises one or more polyols in addition to glycerin, e.g., glycerin and propylene glycol. Without being bound to any theory, mixing propylene glycol and glycerin may improve the entrapment of the resulting polar phase in the hydrophobic phase beyond that provided by propylene glycol alone, while also taking advantage of the superior tetracycline solubility benefits of propylene glycol over glycerin. This may be beneficial to increase the overall solubility of tetracycline antibiotic in the composition, since propylene glycol has a higher solubility of tetracycline antibiotic as compared to glycerin alone. In some embodiments, the composition comprises about 0.1, 0.5, 1, 2, 3, 4, 5, 6, 7, 8, 9, 10, 15, 20, 25% or more or any ranges between any two figures listed by weight of the composition of propylene glycol. In some other embodiments, the composition comprises less than about 0.1, 0.5, 1, 2, 3, 4, 5, 6, 7, 8, 9, 10, 15, 20, 25% or more or any ranges between any two figures listed by weight of the composition of propylene glycol.

In some embodiments, the hydrophobic phase in the composition comprises a hydrophobic solvent and a wax, or a hydrophobic solvent, a wax and a thickened silicone, or a wax and a thickened silicone. The hydrophobic phase may be present at about 40% to about 90%, about 60% to about 85%, about 70% to about 85%, or about 75% to about 85% by weight of the composition, or any percentage in between. In some embodiments, the hydrophobic phase is present in the composition at about 50%, about 60%, about 65%, about 70%, about 75%, about 80%, about 85% or about 90% by weight of the composition or any ranges between any two figures listed. In some embodiments, the wax comprises a petroleum wax. In some embodiments, the wax comprises a micro-crystalline wax. In some embodiments, the wax comprises a mineral wax. In some embodiments the wax comprises a beeswax. In some embodiments, the melting point of the wax is below about 65° C., e.g., between about 64° C. and about 42° C. In some embodiments, the melting point of the wax is below about 42° C., e.g., between about 41° C. and about 25° C. In some embodiments, the wax comprises a mixture of two or more waxes in order to achieve a certain melting temperature. In some embodiments, the wax is a paraffin wax. Paraffin wax may be present at about 25%, about 30%, about 35%, about 40%, about 45%, or about 50% by weight of the composition. Paraffin wax may be present at about 40% by weight of the composition. In some embodiments, paraffin wax is paraffin wax 42-44, paraffin wax 51-53, or paraffin wax 57-60. In some embodiments, paraffin wax is paraffin wax 57-60.

In some embodiments, the hydrophobic solvent comprises a mineral oil and/or a medium chain triglyceride oil. In some embodiments, the hydrophobic solvent is a mineral oil. Mineral oil may be present in the composition at about 2% to about 75%. In one or more embodiments, the composition comprises a hydrophobic solvent in a concentration of about 27% to about 73% by weight, about 27% to about 35% by weight, about 35% to about 40% by weight, about 40% to about 45% by weight, about 45% to about 50% by weight, about 50% to about 55% by weight, about 55% to about 60% by weight, about 60% to about 65% by weight, about 65% to about 70% by weight, and about 70% to about 73% by weight of the composition. In one or more embodiments, the composition comprises a hydrophobic solvent in a concentration of about 27% by weight, or about 30% by weight, or about 35% by weight, or about 40% by weight, or about 45% by weight, or about 50% by weight, or about 55% by weight, or about 60% by weight, or about 65% by weight, or about 70% by weight, or about 73% by weight of the composition or any ranges between any two figures listed.

In one or more embodiments, the mineral oil may be present in the composition at about 25%, or at about 37% by weight of the composition, or at a range between them.

In some embodiments, a composition described herein comprises (i) a polar phase comprising a polar solvent and (ii) a hydrophobic phase comprising a hydrophobic solvent, and a wax or a hydrophobic solvent, a wax and a thickened silicone, or a wax and a thickened silicone. In some embodiments, the ratio between the hydrophobic phase and polar phase is about 4.5:1, 4:1, 3.5:1, 3:1, 2.5:1, 2:1, 1.5:1, or 1:1. In some embodiments, the ratio between the hydrophobic phase and polar phase is about 3.5:1. In some embodiments, the ratio between the wax and polar solvent is about 1:1, 1.5:1,1.8:1, 2:1, or 2.5:1. In some embodiments, the ratio between the wax and polar solvent is about 2: 1. In some embodiments, the ratio between the wax and hydrophobic solvent is about 0.5:1, 0.75:1, 1:1, or 1:1.5. In some embodiments, the ratio between the wax and hydrophobic solvent is about 1:1. In some embodiments, the ratio between the polar solvent and hydrophobic solvent is about 1:1, 1:1.2, 1:1.5, 1:1.8, or 1:2. In some embodiments, the ratio between the polar solvent and hydrophobic solvent is about 1:1.8.

In some embodiments, the polar solvent is glycerin. In some embodiments, the polar solvent also comprises propylene glycol. In some embodiments, the hydrophobic solvent comprises a mineral oil. In some embodiments, the hydrophobic solvent comprises a plant oil. In some embodiments, the wax comprises a paraffin wax.

In some embodiment, the composition comprises about 15-25% by weight of glycerin and optionally up to about 10% by weight of propylene glycol, about 30-40% by weight of mineral oil, and about 30-50% by weight of paraffin wax 57-60. In some embodiment, the composition comprises about 20% by weight of glycerin, about 37% by weight of mineral oil, and about 40% by weight of paraffin wax 57-60.

### Polar Phase

In various embodiments, the polar phase disclosed herein comprises one or more polar (or hydrophilic) solvent. The polar phase is provided to solubilize the API. In accordance with those embodiments, the API is solubilized within the polar phase, which is entrapped within the hydrophobic phase.

In some embodiments, the polar phase is present in the composition at about 10% to about 75% by weight of the composition, about 15% to about 70% by weight of the composition, about 15% to about 65% by weight of the composition, about 15% to about 60% by weight of the composition, about 1% to about 40% by weight of the composition, about 5% to about 35% by weight of the composition, about 15% to about 30% by weight of the composition, about 15% to about 25% by weight of the composition, about 15% to about 20% by weight of the composition, or any percentage in between. In some embodiments, the polar solvent is present in the composition at about 15%, about 20%, about 25%, about 30%, about 35%, about 40%, about 45%, about 50%, about 55%, about 60%, about 65%, about 70%, or about 75% by weight of the composition. In one or more embodiments the composition comprises a polar solvent in a concentration of about 5% to about 35% by weight of the composition. For e.g. about 5% to about 10% by weight, about 10% to about 15% by weight, about 15% to about 20% by weight, about 20% to about 25% by weight, about 25% to about 30% by weight or about 30% to about 35% by weight of the composition.

### Polyols

Polyols are examples of polar solvents that can be contained in the carriers provided herein. A polyol is an organic substance that contains at least two hydroxy groups in its molecular structure.

In one or more embodiments, the carrier comprises at least one diol (i.e., a compound that contains two hydroxy groups in its molecular structure). In one or more embodiments, the carrier comprises at least one monohydric alcohol (i.e., a compound that contains one hydroxy group in its molecular structure). In one or more embodiments, the monohydric alcohol is a propanol. In one or more embodiments, the monohydric alcohol comprises one or more of methanol, ethanol, propanol, butanol, pentanol and isopropanol. In one or more embodiments, the carrier is substantially free, essentially free or free of a diol. In one or more embodiments, the diol is a glycol. In one or more embodiments, the glycol is propylene glycol and/or hexylene glycol. Examples of diols include propylene glycol (e.g., 1,2-propylene glycol and 1,3-propylene glycol), butanediol (e.g., 1,2-butanediol, 1,3-butanediol, 2,3-butanediol and 1,4-butanediol), butanediol (e.g., 1,3-butanediol and 1,4-butenediol), butynediol, pentanediol (e.g., pentane-1,2-diol, pentane-1,3-diol, pentane-1,4-diol, pentane-1,5-diol, pentane-2,3-diol and pentane-2,4-diol), hexanediol (e.g., hexane-1,6-diol hexane-2,3-diol and hexane-2,56-diol), hexylene glycol, octanediol (e.g., 1,8-octanediol), neopentyl glycol, 2-methyl-1,3-propanediol, diethylene glycol, triethylene glycol, tetraethylene glycol, dipropylene glycol, and dibutylene glycol.

In one or more embodiments, the carrier contains at least one triol (a compound that contains three hydroxy groups in its molecular structure), such as glycerin, butane-1,2,3-triol, butane-1,2,4-triol, and hexane-1,2,6-triol.

In one or more embodiments, a polyol refers to a mixture of polyols. In one or more embodiments, the mixture of polyols may contain at least one diol and at least one triol. In one or more embodiments, the mixture of polyols may contain at least two diols or at least triols. In one or more embodiments, the polyol is selected from glycerin, hexylene glycol, and propylene glycol. In one or more embodiments, the polyol is glycerin. In yet one or more embodiments, the polyol is hexylene glycol. In yet one or more embodiments, the polyol is propylene glycol. In yet one or more embodiments, the polyol is propylene glycol and glycerin. In yet one or more embodiments, the polyol is hexylene glycol and glycerin. In yet one or more embodiments, the polyol is a combination of propylene glycol and glycerin.

In some embodiments, the polar phase comprises glycerin. In some embodiments, glycerin is present in the composition at about 15% to about 45% by weight of the composition, about 15% to about 35% by weight of the composition, about 1% to about 40% by weight of the composition, about 5% to about 35% by weight of the composition, about 5% to about 40% by weight of the composition, about 5% to about 35% by weight of the composition, about 15% to about 30% by weight of the composition, about 15% to about 25% by weight of the composition, about 15% to about 20% by weight of the composition, or any percentage in between. In some embodiments, glycerin is present in the composition at about 18% to about 22% by weight of the composition. In some embodiments, glycerin is present in the composition at about 20% by weight of the composition.

In some embodiments, the polar phase comprises propylene glycol. In some embodiments, the polar phase comprises hexylene glycol. In some embodiments, propylene glycol or hexylene glycol may be present in the composition at about 10% to about 35% by weight of the composition, about 15% to about 30% by weight of the composition, about 15% to about 25% by weight of the composition, about 15% to about 20% by weight of the composition, or any percentage in between.

In one or more embodiments, the amount of glycerin is about or below about 35%, about or below about 30%, about or below about 25%, about or below about 20%, about or below about 15%, about or below about 10%, or about or below about 5%.

In an exemplary embodiment, the polyol is a combination of propylene glycol and glycerin. In yet one or more embodiments, the propylene glycol is present at about 15% to about 30% and glycerin is present at about 15% to about 35% by weight. In yet one or more embodiments, the propylene glycol is present at about 20% to about 27% and glycerin is present at about 20% to about 30% by weight.

In a further exemplary embodiment, the polyol is a combination of hexylene glycol and glycerin. In yet one or more embodiments, the hexylene glycol is present at about or more than about 21.5% and glycerin is present at about or more than about 29% by weight.

Another class of polyols includes polyethylene glycols (“PEGs”). Exemplary PEGs include, without limitation, PEG 200, PEG 300, PEG 400, PEG 600, PEG 1000, PEG 4000, PEG 6000, and PEG 8000. The carrier according to the present invention can contain a single PEG or a mixture of two or more PEGs. In some embodiments, the polyol comprises a liquid PEG having a molecular weight of about 600 or less. In some embodiments, the polyol comprises a solid PEG having a molecular weight of about 1000 or more. Solid PEGs can contribute to providing an ointment type composition and to an increased viscosity. In one or more embodiments the composition is substantially free, essentially free, or free of a PEG.

In one or more embodiments, the polar phase may comprise a polar humectant or a polar moisturizer such as a polyol, a polyethylene glycol, a polypropylene glycol, urea, and/or lactic acid.

### Aprotic Polar Solvents

An aprotic solvent is an organic solvent that does not contain an O—H or N—H bond; or does not exchange protons with a substance dissolved in it. In the context herein, the aprotic polar solvent is a solvent with a comparatively high relative permittivity (or dielectric constant), greater than about 15, and a sizable permanent dipole moment, that cannot donate suitably labile hydrogen atoms to form strong hydrogen bonds; and it is miscible in water. Examples of aprotic polar solvents, suitable according to the present invention include, but are not limited to dimethyl sulfoxide (DMSO), dimethylformamide (DMF), acetonitrile, acetone, methyl ethyl ketone, 1,4-Dioxane and tetrahydrofuran (THF). Additional non-limiting examples include N-methylpyrrolidone, pyridine, piperidine, dimethyl ether, hexamethylphosphorotriamide, dimethylformamide, methyl dodecyl sulfoxide, N-methyl-2-pyrrolidone and 1-methyl-2-pyrrolidinone) and azone (1-dodecylazacycloheptan-2-one).

### Hydrophobic Phase

The herein disclosed compositions may optionally include a hydrophobic phase within which the polar phase is stabilized and entrapped. In some embodiments, the hydrophobic phase is present in the composition at about 20% to about 95% by weight of the composition, about 20% to about 90%, about 30% to about 95%, about 35% to about 90%, about 40% to about 90%, about 60% to about 85%, about 70% to about 85%, or about 75% to about 85% by weight of the composition, or any percentage in between. In some embodiments, the hydrophobic phase is present in the composition at about 50%, about 60%, about 70%, about 75%, about 80% or about 85% by weight of the composition.

In one or more embodiments, the hydrophobic phase may comprise a hydrophobic emollient. In one or more embodiments, the hydrophobic emollient includes or is selected from avocado oil, isopropyl myristate, a mineral oil, a capric triglyceride, a capryllic triglyceride, isopropyl palmitate, isopropyl isostearate, diisopropyl adipate, diisopropyl dimerate, a maleated soybean oil, octyl palmitate, cetyl lactate, cetyl ricinoleate, tocopheryl acetate, an acetylated lanolin alcohol, cetyl acetate, phenyl trimethicone, glyceryl oleate, tocopheryl linoleate, wheat germ glycerides, arachidyl propionate, myristyl lactate, decyl oleate, ricinoleate, isopropyl lanolate, pentaerythrityl tetrastearate, neopentylglycol dicaprylate/dicaprate, isononyl isononanoate, isotridecyl isononanoate, myristyl myristate, triisocetyl citrate, octyl dodecanol, unsaturated or polyunsaturated oils, an olive oil, a corn oil, a soybean oil, a canola oil, a cottonseed oil, a coconut oil, a sesame oil, a sunflower oil, a borage seed oil, a syzigium aromaticum oil,a hempseed oil, a herring oil, a cod-liver oil, a salmon oil, a flaxseed oil, a wheat germ oil, a evening primrose oil, an essential oil, a silicone oil, dimethicone, cyclomethicone, polyalkyl siloxane, polyaryl siloxane, polyalkylaryl siloxane, a polyether siloxane copolymer, a poly(dimethylsiloxane)-(diphenyl-siloxane), and a mixture of any two or more thereof.

In one or more embodiments, the ratio of the hydrophobic phase to polar phase is between about 9:1 to 1:9, 8:1 to 1:8, 7:1 to 1:7, 6:1 to 1:6, 5:1 to 1:5, 4:1 to 1:4, 3:1 to 1:3, or 2:1 to 1:2. In one or more embodiments, the ratio of the hydrophobic phase to polar phase is between about 1:1 to 10:1, 1:1 to 9:1, 1:1 to 8:1, 1:1 to 7:1, 1:1 to 6:1, 1:1 to 5:1, 1:1 to 4:1, 1:1 to 3:1, or 1:1 to 2:1. In one or more embodiments, the ratio between the hydrophobic phase comprising a liquid oil and a wax, to hydrophilic phase in the herein disclosed compositions is more than about 1.5:1. For e.g. more than about 1.6:1, more than about 1.7:1, more than about 1.8:1, more than about 1.9:1, about or more than about 2:1, about or more than about 2.5:1, about or more than about 3:1, about or more than about 3.5:1, about or more than about 4:1, about or more than about 5:1, about or more than about 6:1, about or more than about 7:1, about or more than about 8:1, about or more than about 9:1, about or more than about 10:1. In one or more embodiments, the ratio of the hydrophobic phase to polar phase is between about 1.85:1 to about 18.88:1, about 1.85:1 to about 5.5:1, about 1.85:1 to 3.88:1, about 2.9:1 to about 5.5:1, or about 3.88:1.

In one or more embodiments the hydrophobic phase comprises a thickened silicone. A thickened silicone is a mixture of silicone oils and high viscosity silicone, which acts as a thickening agent. Examples of thickened silicones include but are not limited to cyclopentasiloxane and dimethicone crosspolymer (ST-Elastomer 10), stearoxytrimethylsilane and stearyl alcohol (Silky Wax 10), dimethicone and polysilicone-11 (Gransil DMG5), Cyclopentasiloxane (and) Polysilicone-11 (MGS-Elastomer 1100), Cyclopentasiloxane (and) Petrolatum (and) Polysilicone-11 (MGS-Elastomer 1148P) and polymethylsilsesquioxane (MGS powder 3300).In one or more embodiments, the thickened silicone has a viscosity of about 40,000 cPs to about 1,500 ,000 cPs. For example, about 40,000 cPs to about 100,000 cPs, or about 50,000 cPs to about 90,000 cPs, or about 40,000 cPs to about 85,000 cPs. For example, about 300,000 cPs to about 1,300 ,000 cPs, or about 400,000 cPs to about 1,200 ,000 cPs, or about 500,000 cPs to about 1,000 ,000 cPs. For example, about 50,000 cPs, or about 60,000 cPs, or about 70,000 cPs, or about 80,000 cPs, or about 90,000 cPs, or about 100,000 cPs, or about 150,000 cPs, or about 200,000 cPs, or about 250,000 cPs, or about 300,000 cPs, or about 350,000 cPs, or about 400,000 cPs, or about 450,000 cPs, or about 500,000 cPs, or about 550,000 cPs, or about 600,000 cPs, or about 650,000 cPs, or about 700,000 cPs, or about 750,000 cPs, or about 800,000 cPs, or about 850,000 cPs, or about 900,000 cPs, or about 950,000 cPs, or about 1,000 ,000 cPs, or about 1,100 ,000 cPs, or about 1,200 ,000 cPs, or about 1,300 ,000 cPs, or about 1,400 ,000 cPs, or about 1,500 ,000 cPs, or about any range between any of the figures described herein.

The hydrophobic phase may include mixtures of two or more hydrophobic substances. In certain embodiments, the hydrophobic phase comprises a mixture of one or more liquid hydrophobic solvents and an additional hydrophobic substance, which is not a liquid at ambient temperature (such as a wax).

### Waxes

In some embodiments, the herein disclosed formulations may comprise a wax. In some embodiments, the wax is solid or semi solid at room temperature. In some embodiments, the wax is solid or semi solid at body temperature.

Exemplary waxes include, but are not limited to, a plant wax, an animal wax, a petroleum, a petroleum derived wax, a mineral wax, a vegetable wax, an albacer wax, an atlasenewax, a cardis wax, a ceramid, a beeswax, a BASF wax, a carnauba wax, a chinese wax, a cotton wax, a bayberry wax, a carnauba wax, a castor wax, a cuban palm wax, a duroxon wax, an emulsifying wax, an esparto wax, a fat wax, a flax wax, a fischer-tropsch wax, a fir wax, a flexo wax, a flower wax, a glyco wax, a japan wax, a jojoba oil, a lanolin wax, a palm wax, a rice bran wax, a rice-oil wax, a shellac wax, a soy wax, an ucuhuba wax, a hydrogenated oil, a hydrogenated castor oil, a hydrogenated cottonseed oil, a hydrogenated jojoba oil, microcrystalline wax, a mink wax, a mixture of saturated n- and isoalkanes, a montan wax, a naphthene, an ouricury wax, an oxazoline wax, an ozokerite, a paraffin wax, a paraffin 58-62° C. wax, paraffin 57-60° C. wax, paraffin 51-53° C. wax, paraffin 42-44° C. wax, a polyethylene, a PEG-6 beeswax, a polyolefin, a polymekon wax, a retamo wax, a rezo wax, a sandy wax, a soy wax, a spent grain wax, a stearyl dimethicone, a sugarcane wax, a mineral wax, or mixtures and any two or more thereof.

In one or more embodiments, the wax is a hydrocarbon-based wax. In one or more embodiments, the wax is selected from a beeswax, a paraffin wax, a microcrystalline wax, or a combination thereof. In an exemplary embodiment, the wax is a combination of two or more type of waxes. For example, the wax is a combination of three or more, or four or more waxes. In an exemplary embodiment, the wax includes beeswax and paraffin wax. In one or more embodiments, the wax comprises a combination of a paraffin wax and a microcrystalline wax. In one or more embodiments, the wax comprises a combination of a paraffin wax and an emulsifying wax. In one or more embodiments, the wax comprises a microcrystalline wax and a beeswax. In one or more embodiments, the wax comprises a microcrystalline wax and an emulsifying wax. In some embodiments the wax is a combination of at least two waxes having a different average of particle or crystal sizes, which can contribute to or improve composition stability over that provided by individual waxes alone.

In one or more embodiments, the wax is substantially free, essentially free or free of hydrogenated castor oil. In one or more embodiments, the wax is substantially free, essentially free or free of a saturated vegetable oil wax. In one or more embodiments, the wax is substantially free, essentially free or free of a saturated oil wax. In one or more embodiments, the wax is substantially free, essentially free or free of a non-hydrocarbon-based wax.

In one or more embodiments, the ratio between the wax and polar solvent in the herein disclosed compositions is more than about 0.88:1, or about or more than about 2:1.In some embodiments, the ratio between the wax and polar solvent in the herein disclosed compositions is more than about 0.5:1. For e.g. about or more than 0.6:1, about or more than 0.7:1, about or more than about 0.8:1, about or more than about 0.9:1, about or more than about 1:1 about or more than about 1:1.2. In some embodiments, the ratio between the wax and polar solvent in the herein disclosed compositions is about or more than about 1.5:1, about or more than about 2:1, about or more than about 2.5:1, about or more than about 3:1, about or more than about 3.5:1, about or more than about 4:1, about or more than about 4.5:1, about or more than about 5:1.

In some embodiments, the wax is a paraffin wax. In some embodiments, the paraffin wax is selectedfrom paraffin 57-60° C. wax, 56-58° C. wax, paraffin 52-54° C. wax, paraffin 51-53° C. wax, 46-48° C. wax, paraffin 42-44° C. wax and mixtures of any two or more thereof.

In some embodiments, the wax is solid or semi solid at room temperature having a melting point of at least about 125° F., at least about 127° F., at least about, 130° F., at least about 135° F., at least about 140° F., at least about 145° F., at least about 150° F., at least about 155° F., at least about 160° F., or at least about 165° F.

In some embodiments, the wax is solid or semi solid at room temperature having a melting point of at least about 25° C., or at least about 30° C., or at least about, 35° C., or at least about 40° C., or at least about 42° C., or at least about 44° C., or at least about 45° C., or at least about 50° C., or at least about 55° C., or at least about 60° C., or at least about 65° C., or at least about 70° C.

In some embodiments, the wax has a melting point range of 125° F. to 165° F.; 135° F. to 150° F.; or 150° F. to 165° F. or any other similar or relative range(s) or mixtures thereof. In some embodiments, the wax has a melting point range of 25-70° C.; 30-65° C.; 35-65° C.; or 40-65° C. or any other similar or relative range(s) or mixtures thereof.

In some embodiments, the wax has a melting point ranges of 125° F. to 135° F.; 127° F. to 130° F.; 130° F. to 135° F.; 135° F. to 145° F.; 140° F. to 145° F.; 150° F. to 155° F.; 150° F. to 165° F.; 160° F. to 165° F.; or such as 43-46° C.; 46-53° C.; 48-50° C.; 52-54° C.; 53-55° C.; 54-57° C.; 54-58° C.; 58-60° C.; 59-61° C.; 60-62° C.; 62-66° C.; 62-65° C.; 65-68° C.; or any other similar or relative range(s) or mixtures thereof.

In some embodiments, the wax can be a combination of a high melting point wax and a medium or low melting point wax, with the proviso that the average melting point of the wax combination is below 65° C., below 60° C., below 59° C., or below 58° C.

In some embodiments, the wax is present in the composition at above about 1% and below about 60%. In some embodiments, the wax is present in the composition at about 5% to about 65% by weight of the composition, about 10% to about 60%, about 15% to about 60%, about 20% to about 60%, about 25% to about 60%, about 25% to about 55%, about 25% to about 50%, about 30% to about 55%, about 30% to about 50%, about 5% to about 40%, about 3% to about 45%, about 5% to about 35%, or about 10% to about 40%, by weight of the composition, or any percentage in between. In some embodiments, the wax is present in the composition at about 25%, about 30%, about 35% or about 40% by weight of the composition. In one or more embodiments, the composition comprises a wax in a concentration of about 5% to about 50% by weight of the composition. For e.g. about 5% to about 10% by weight, about 10% to about 15% by weight, about 15% to about 20% by weight, about 20% to about 25% by weight, about 25% to about 30% by weight, about 30% to about 35% by weight, about 35% to about 40% by weight, about 40% to about 45% by weight, about 45% to about 50% by weight, about 5% to about 15% by weight, about 15% to about 25% by weight, about 25% to about 35% by weight, about 35% to about 45% by weight, and about 40% to about 50% by weight of the composition. In one or more embodiments the composition comprises a wax in a concentration of about 5% by weight of the composition or about 10% by weight, or about 15% by weight, or about 20% by weight, or about 25% by weight, or about 30% by weight, or about 35% by weight, or about 40% by weight, or about 45% by weight, or about 50% by weight of the composition.

In one or more embodiments, the composition comprises more than about 5% by weight of wax. For example, more than about 10% by weight of wax, more than about 15% by weight of wax, more than about 20% by weight of wax, more than about 25% by weight of wax, more than about 30% by weight of wax, more than about 35% by weight of wax, more than about 40% by weight of wax, more than about 45% by weight of wax, more than about 50% by weight of wax, or more than about 55% by weight of wax. In one or more embodiments, the composition comprises about 10% to about 55% wax by weight of the composition. For example, about 10% to about 20%, about 20% to about 30%, about 30% to about 40%, about 40% to about 50% or about 45% to about 55% of wax by weight of the composition. In one or more embodiments, the ratio between the wax and the hydrophobic solvent is about 1:10 to about 1:0.1. For e.g. about 1:9, or about 1:8, or about 1:7, or about 1:6, or about 1:5, or about 1:4, or about 1:3, or about 1:2, or about 1:1, or about 1:0.5, or about 1:0.3. In one or more embodiments, the ratio between the wax and the hydrophobic solvent is about 1:3 to about 1:0.6. In one or more embodiments, the ratio between the wax and the hydrophobic solvent is about 1:3 or more than about 1:3. In one or more embodiments, the ratio between the wax and the hydrophobic solvent is about 1:8 to about 1:0.6. In one or more embodiments, the ratio between the wax and the hydrophobic solvent is about 1:8 or more than about 1:8. In certain embodiments,, the ratio of the wax to hydrophobic solvent to is between about 0.07:1 to about 1.22:1, about 0.44:1 to about 1:1.22, about 0.15:1 to about 1.22:1, about 0.93:1 to about 1.22:1, or about 1.06:1. In other embodiments, the ratio between the wax and the hydrophobic solvent in the composition is less than about 1.8:1. For example, less than about 1.44:1, or is about 1.22:1 or less than about 1.22:1.

In some embodiments, the hydrophobic phase comprises a solid or semi solid fatty alcohol or fatty acid. In some embodiments, the hydrophobic phase comprises a long chain fatty alcohol, a long chain fatty acid or mixtures of both. In some embodiments, the composition is substantially free of a fatty acid and/or a fatty alcohol. In some embodiments, the composition is essentially free of a fatty acid and/or a fatty alcohol. In some embodiments, the composition is free of a fatty acid and/or a fatty alcohol. In some embodiments, the composition is substantially free of a solid or semi solid fatty acid and/or a fatty alcohol. In some embodiments, the composition is essentially free of a solid or semi solid fatty acid and/or a fatty alcohol. In some embodiments, the composition is free of a solid or semi solid fatty acid and/or a fatty alcohol. Unless otherwise stated the terms solid and semi-solid refer to specific ingredients or the composition at room temperature. In one or more other embodiments, the herein disclosed formulations comprise one or more fatty alcohols having less than 22 carbon atoms in the backbone. For example, the one or more fatty alcohols include 20 or less than 20, 18 or less than 18 carbon atoms in the backbone chain. In one or more embodiments, the herein disclosed formulations comprise one or more fatty alcohols having between 14 and 20 or between 14 and 18, or between 16 and 18, or between 16 and 22 carbon atoms in the backbone chain. In one or more embodiments, the fatty alcohol is myristyl alcohol and/or stearyl alcohol. In one or more embodiments, the fatty alcohol is substantially free, essentially free or free of behenyl alcohol. In one or more embodiments, the fatty alcohol comprises a combination of fatty alcohols comprising a fatty alcohol having between 14 and 20 carbon atoms in its backbone and a fatty alcohol having at least 20 carbon atoms in its backbone. In one or more embodiments, the fatty alcohol comprises a combination of any of the fatty alcohols mentioned herein. See, e.g., the section below describing fatty alcohols. In one or more embodiments, the fatty alcohol comprises a combination with any of the fatty acids mentioned herein, for example stearic acid.

In one or more embodiments, hydrophobic phase comprises a combination of a hydrophobic solvent comprising a liquid oil, at least one wax, including a fatty alcohol and or a fatty acid and may also comprise semi-solid hydrocarbon like petrolatum and or a thickened silicone. In one or more embodiments the hydrophobic phase is suitable to be applied topically to the skin or mucosa and may include an active pharmaceutical agent. In one or more embodiments the hydrophobic phase is combined with a polar phase to form a homogenous composition suitable to be applied topically to the skin or mucosa and the polar phase may include an active pharmaceutical agent.

In one or more embodiments, the ratio between the wax to polar solvent is about 0.25:1 to about 8:1. For example, about 0.25:1 to about 2:1, about 0.5:1 to about 2.66:1, or about 1.6:1 to about 2:1. In an exemplary embodiment, the ratio between the wax to polar solvent is about 2:1.

### Fatty Alcohols

A long chain fatty alcohol as used herein has above 8 carbon atoms in its carbon chain. A long chain fatty alcohol as used herein may have according to some embodiments 8 to 22 or 14 to 22 carbon atoms in its carbon chain. In some embodiments, the long chain fatty alcohol has 16 to 22 carbon atoms in its carbon chain. In some embodiments, the long chain fatty alcohol is selected from fatty alcohols having 15 or more carbons in their carbon chain, such as cetyl alcohol and stearyl alcohol (or mixtures thereof, i.e., cetostearyl alcohol). Other examples of long chain fatty alcohols are myristyl alcohol (C14), arachidyl alcohol (C20), and behenyl alcohol (C22). In some embodiments, the fatty alcohol is cetyl alcohol, stearyl alcohol, behenyl alcohol, myristyl alcohol, or mixtures of two or more thereof. In some embodiments, the long chain fatty alcohol is cetostearyl alcohol. In some embodiments, the fatty alcohol chain is saturated. In some embodiments, it can be unsaturated. In some embodiments, the long chain fatty alcohol is branched. In some embodiments, the long chain fatty alcohol is a mixture of two or more fatty alcohols. In one or more further embodiments, the composition comprises a medium chain fatty alcohol having a chain length of C14 or below C14, for example, a fatty alcohol having C14 to C8 carbon atoms in its carbon chain. In one or more still further embodiments, the composition can comprise very long chain fatty alcohols having a carbon chain of more than C22, for example, in a fatty alcohol having C24 to C50 carbon atoms in its carbon chain. In certain embodiments the wax comprises a fatty alcohol. For e.g. stearyl alcohol. In one embodiment, the ratio of the wax (for e.g. fatty alcohol): hydrophobic solvent is between about 0.3:1 to about 1:1. For e.g. about 0.5:1, or about 0.6:1, or about 0.7:1, or about 0.8:1, or about 0.9:1.

In one embodiment, the ratio of the wax (e.g., fatty alcohol): hydrophobic solvent is between about 0.25:1 to about 1.2:1, or between about 0.5:1 and about 1:1, or between about 0.35:1 to about 1.06:1, or between about 0.6:1 and about 1:1.

In one or more embodiments the amount of fatty alcohol is between about 5% to about 45%, or is between about 15% to about 45%, or is between about 10% to about 40%, or. For e.g. about 5%, or about 8%, or about 12%, or about 15%, or about 18%, or about 20%, or about 25%, or about 30%, or about 35%, or about 40%, or between any range between any of the aforesaid amounts. In one or more embodiments the amount of fatty alcohol is between about 20% to about 40%.

In one or more embodiments, the fatty alcohol comprises a solid fatty alcohol. In one or more embodiments, the fatty alcohol comprises a liquid fatty alcohol. In one or more embodiments, the liquid fatty alcohol comprises octyl dodecanol. In one or more embodiments, the liquid fatty alcohol comprises oleyl alcohol. In one or more embodiments, the fatty alcohol is substantially free, essentially free or free of solid fatty alcohols. In one or more embodiments, the fatty alcohol is substantially free, essentially free or free of liquid fatty alcohols. In one or more embodiments, the fatty alcohol is substantially free, essentially free or free of octyl dodecanol. In one or more embodiments, the fatty alcohol is substantially free, essentially free or free of oleyl alcohol.

### Fatty Acids

In some embodiments, the fatty acid can be a straight chain fatty acid, a saturated fatty acid, an unsaturated fatty acid, a hydroxyl fatty acid, or a branched fatty acid.

In some embodiments, the fatty acid is a fatty acid having 16 or more carbons in its carbon chain, such as hexadecanoic acid (C16) stearic acid (C18), arachidic acid (C20), behenic acid (C22), octacosanoic acid (C28), as well as fatty acids with longer carbon chains (up to C50), or mixtures thereof. In some embodiments, the fatty acid is selected from fatty alcohols having 14 or less carbons in their carbon chain, such as myristic acid, myristoleic acid, and lauric acid.

In some embodiments, the composition is free, or essentially free, or substantially free of solid fatty alcohols or solid fatty acids. In some embodiments, the composition is free, or essentially free, or substantially free of fatty alcohols having 15 or more carbons in its carbon chain or having 15 to 22 carbons in its carbon chain. In some embodiments, the composition is free, or essentially free, or substantially free of fatty acids having 16 or more carbons in its carbon chain or having 16 to 24 carbons in its carbon chain. Whilst certain formulations can be stable without apparent phase separation in the absence of fatty alcohols and/or fatty acids, the presence of small quantities may aid further stabilization of certain formulations, e.g., formulations comprising a mixture of diols and triols (see Examples). In some other embodiments, the fatty alcohol or fatty acid may be present in the composition at a low concentration of between about 0.1% to about 5% by weight of the composition. In yet other embodiments, the fatty alcohol or fatty acid may be present in the composition at a higher concentration of between about 5% to about 60%, about 10% to about 50%, about 15% to about 50%, about 20% to about 50%, about 25% to about 50%, about 30% to about 50%, or about 35% to about 50% by weight of the composition. When present in higher concentrations, some of the fatty alcohols and/fatty acids are preferably liquid or liquid mixtures so that solid fatty acids and/or solid fatty alcohols are dissolved or dispersed in liquid fatty acids and or liquid fatty alcohols. According to some embodiments, the fatty alcohol and/or fatty acid is present in the composition at a concentration of at least about 15%, at least about 20%, at least about 25% at least about 30%, at least about 35%, at least about 37%, at least about 38%, at least about 39%, or at least about 40% by weight of the composition. According to some embodiments, the fatty alcohol or fatty acid is present in the composition at a concentration of about 35% w/w, about 37% w/w, about 38% w/w, about 39% w/w, or about 40% w/w.

### Hydrophobic Solvent

In some embodiments, the herein disclosed formulations comprise a hydrophobic solvent. In one or more embodiments the hydrophobic solvent is liquid. In some embodiments the hydrophobic solvent may be an oil. In some embodiments the hydrophobic solvent is a mixture of a liquid oil and a solid oil and or a semi-solid oil. In one or more embodiments, the mixture of oils remains as a liquid. Exemplary oils include, without limitation, a mineral oil, a hydrocarbon oil, an ester oil, an ester of a dicarboxylic acid, a triglyceride oil, an oil of plant origin, a plant oil rich in triglycerides, an oil from animal origin, an unsaturated or polyunsaturated oil, a diglyceride, a PPG alkyl ether, an essential oil, a silicone oil, liquid paraffin, a paraffin, a polyalphaolefin, a polyolefin, polyisobutylene, a synthetic isoalkane, isohexadecane, isododecane, alkyl benzoate, alkyl octanoate, C12-C15 alkyl benzoate, C12-C15 alkyl octanoate, arachidyl behenate, arachidyl propionate, benzyl laurate, benzyl myristate, benzyl palmitate, bis (octyldodecyl stearoyl) dimer dilinoleate, butyl myristate, butyl stearate, cetearyl ethylhexanoate, cetearyl isononanoate, cetyl acetate, cetyl ethylhexanoate, cetyl lactate, cetyl myristate, cetyl octanoate, cetyl palmitate, cetyl ricinoleate, decyl oleate, diethyleneglycol diethylhexanoate, diethyleneglycol dioctanoate, diethyleneglycol diisononanoate, diethyleneglycol diisononanoate, diethylhexanoate, diethylhexyl adipate, diethylhexyl malate, diethylhexyl succinate, diisopropyl adipate, diisopropyl dimerate, diisopropyl sebacate, diisosteary dimer dilinoleate, diisostearyl fumerate, dioctyl malate, dioctyl sebacate, dodecyl oleate, ethylhexyl palmitate, ester derivatives of lanolic acid, ethylhexyl cocoate, ethylhexyl ethylhexanoate, ethylhexyl hydroxystarate, ethylhexyl isononanoate, ethylhexyl palmytate, ethylhexyl pelargonate, ethylhexyl stearate, hexadecyl stearate, hexyl laurate, isoamyl laurate, isocetyl behenate, isocetyl lanolate, isocetyl palmitate, isocetyl stearate, isocetyl salicylate, isocetyl stearate, isocetyl stearoyl stearate, isocetearyl octanoate, isodecyl ethylhexanoate, isodecyl isononanoate, isodecyl oleate, isononyl isononanoate, isodecyl oleate, isohexyl decanoate, isononyl octanoate, isopropyl isostearate, isopropyl lanolate, isopropyl laurate, isopropyl myristate, isopropyl palmitate, isopropyl stearate, isostearyl behenate, isosteary citrate, isostearyl erucate, isostearyl glycolate, isostearyl isononanoate, isostearyl isostearate, isostearyl lactate, isostearyl linoleate, isostearyl linolenate, isostearyl malate, isostearyl neopentanoate, isostearyl palmitate, isosteary salicylate, isosteary tartarate, isotridecyl isononanoate, isotridecyl isononanoate, lauryl lactate, medium chain triglycerides (MCT), myristyl lactate, myristyl myristate, myristyl neopentanoate, myristyl propionate, octyldodecyl myristate, neopentylglycol dicaprate, octyl dodecanol, octyl stearate, octyl palmitate, octyldodecyl behenate, octyldodecyl hydroxystearate, octyldodecyl myristate, octyldodecyl stearoyl stearate, oleyl erucate, oleyl lactate, oleyl oleate, petrolatum, propyl myristate, propylene glycol myristyl ether acetate, propylene glycol dicaprate, propylene glycol dicaprylate, maleated soybean oil, stearyl caprate, stearyl heptanoate, stearyl propionate, tocopheryl acetate, tocopheryl linoleate, glyceryl oleate, tridecyl ethylhexanoate, tridecyl isononanoate, triisocetyl citrate, an alexandria laurel tree oil, an avocado oil, an apricot stone oil, a barley oil, a borage seed oil, a calendula oil, a canelle nut tree oil, a canola oil, caprylic/capric triglycerides, a castor oil, a coconut oil, a corn oil, a cotton oil, a cottonseed oil, an evening primrose oil, a flaxseed oil, a groundnut oil, a hazelnut oil, glycereth triacetate, glycerol triheptanoate, glyceryl trioctanoate, glyceryl triundecanoate, a hempseed oil, a jojoba oil, a lucerne oil, a maize germ oil, a marrow oil, a millet oil, a neopentylglycol dicaprylate/dicaprate, an olive oil, a palm oil, a passionflower oil, pentaerythrityl tetrastearate, a poppy oil, propylene glycol ricinoleate, a rapeseed oil, a rye oil, a safflower oil, a sesame oil, a shea butter, a soya oil, a soybean oil, a sweet almond oil, a sunflower oil, a sysymbrium oil, a syzigium aromaticum oil, a tea tree oil, a walnut oil, wheat germ glycerides, a wheat germ oil, a PPG-2 butyl ether, a PPG-4 butyl ether, a PPG-5 butyl ether, a PPG-9 butyl ether, a PPG-12 butyl ether, a PPG-14 butyl ether, a PPG-15 butyl ether, a PPG-15 stearyl ether, a PPG-16 butyl ether, a PPG-17 butyl ether, a PPG-18 butyl ether, a PPG-20 butyl ether, a PPG-22 butyl ether, a PPG-24 butyl ether, a PPG-26 butyl ether, a PPG-30 butyl ether, a PPG-33 butyl ether, a PPG-40 butyl ether, a PPG-52 butyl ether, a PPG-53 butyl ether, a PPG-10 cetyl ether, a PPG-28 cetyl ether, a PPG-30 cetyl ether, a PPG-50 cetyl ether, a PPG-30 isocetyl ether, a PPG-4 lauryl ether, a PPG-7 lauryl ether, a PPG-2 methyl ether, a PPG-3 methyl ether, a PPG-3 myristyl ether, a PPG-4 myristyl ether, a PPG-10 oleyl ether, a PPG-20 oleyl ether, a PPG-23 oleyl ether, a PPG-30 oleyl ether, a PPG-37 oleyl ether, a PPG-40 butyl ether, a PPG-50 oleyl ether, a PPG-11 stearyl ether, a herring oil, a cod-liver oil, a salmon oil, a cyclomethicone, a dimethyl polysiloxane, a dimethicone, an epoxy-modified silicone oil, a fatty acid-modified silicone oil, a fluoro group-modified silicone oil, a methylphenylpolysiloxane, phenyl trimethicone, a polyether group-modified silicone oil, and mixtures of any two or more thereof.

In one or more embodiments, the oil is a combination of two or more, three or more or four or more oils. In one or more embodiments, the oil is a hydrocarbon oil. In some embodiments, the oil is or comprises a mineral oil. In one or more embodiments, the oil is selected from mineral oil, octyl dodecanol, caprylic/capric triglycerides, a silicone, a cyclomethicone, a dimethicone, petrolatum and mixtures of any two or more thereof. In one or more embodiments, the oil comprises a combination of mineral oil, octyl dodecanol, and caprylic/capric triglycerides. In one or more embodiments, the oil comprises a combination of mineral oil, cyclomethicone, and caprylic/capric triglycerides. In one or more embodiments, the oil comprises a cyclomethicone and or a dimethicone. In one or more embodiments, the oil comprises petrolatum. In one or more embodiments, the oil comprises a combination of mineral oil and medium chain triglycerides (MCT). In one or more embodiments, the oil is a combination of mineral oil and isopropyl myristate. In one or more embodiments, in cases where the composition includes one type of an oil, the oil is substantially free, essentially free or free of isopropyl myristate. In one or more embodiments, the oil is substantially free, essentially free or free of a soybean oil. In one or more embodiments, the oil is substantially free, essentially free or free of a silicone oil. In one or more embodiments, the oil is substantially free, essentially free or free of a cyclomethicone oil. In one or more embodiments, the oil is substantially free, essentially free or free of a dimethicone oil.

In one or more embodiments, the oil is substantially free, essentially free or free of oil esters such as isopropyl myristate, isopropyl palmitate, and/or isopropyl stearate.

The oil may be present in the composition at a concentration of between about 1% to about 70%, about 1% to about 10%, about 10% to about 20%, about 20% to about 30%, about 30% to about 40%, about 40% to about 50%, about 50% to about 60%, about 5% to about 70%, about 10% to about 70%, about 10% to about 60%, about 15% to about 70%, about 20% to about 70%, about 25% to about 70%, about 30% to about 70%, about 30% to about 75%, or about 35% to about 70% by weight of the composition. According to some embodiments, the oil is present in the composition at a concentration of at least about 15%, at least about 20%, at least about 25% at least about 30%, at least about 35%, at least about 37%, at least about 38%, at least about 39%, at least about 40%, at least about 50%, at least about 55%, at least about 60%, or at least about 70%, by weight of the composition. According to some embodiments, the oil is present in the composition at a concentration of about 35% w/w, about 37% w/w, about 38% w/w, about 39% w/w, about 40% w/w, about 50% w/w, about 55% w/w, about 60% w/w, or about 70% w/w.

In one or more embodiments, the hydrophobic liquid solvent may include a fatty alcohol being liquid at room temperature. In one or more embodiments, the hydrophobic liquid solvent may be a fatty acid being liquid at room temperature. In one or more embodiments, the herein disclosed compositions are substantially free, essentially free or free of a fatty alcohol and/or fatty acid being liquid at room temperature. In one or more embodiments, the hydrophobic liquid solvent may contain a short chain fatty acid, a short chain fatty alcohol, or mixtures thereof, each having up to 8 carbon atoms in its carbon chain. An exemplary liquid fatty acid includes, without limitation, an oleic acid and/or isostearic acid. An exemplary liquid fatty alcohol includes, without limitation, oleyl alcohol, and/or isostearyl alcohol.

In one or more embodiments, the herein disclosed compositions are substantially free, essentially free or free of a long chain liquid fatty alcohol with a hydroxyl group in the middle of the carbon chain, such as octyl dodecanol. In one or more embodiments, the herein disclosed compositions are substantially free, essentially free or free of a long chain liquid fatty alcohol with a double bond in the middle of the carbon chain, such as oleyl alcohol.

The liquid fatty acid or fatty alcohol may be present in the composition at a concentration of between about 1% to about 50%, about 5% to about 50%, about 5% to about 40%, about 15% to about 45%, about 20% to about 45%, about 25% to about 50%, about 30% to about 50%, or about 35% to about 50% by weight of the composition. According to some embodiments, the oil is present in the composition at a concentration of at least about 15%, at least about 20%, at least about 25%, at least about 30%, at least about 35%, at least about 37%, at least about 38%, at least about 39%, or at least about 40% by weight of the composition. According to some embodiments, the oil is present in the composition at a concentration of about 35% w/w, about 37% w/w, about 38% w/w, about 39% w/w, or about 40% w/w.

In one or more embodiments, the ratio of the hydrophobic solvent to wax is between about 9:1 to 1:9, about 8:1 to 1:8, about 7:1 to 1:7, about 6:1 to 1:6, about 5:1 to 1:5, about 4:1 to 1:4, about 3:1 to 1:3, or about 2:1 to 1:2. In one or more embodiments, the ratio of the hydrophobic solvent to polar solvent is between about 1.28:1 to about 10.88:1, about 1.88:1 to about 10.88:1, about 1.28:1 to about 3.38:1, about 2:1 to about 3.63:1, about 1.3:1 to about 2.84:1, or about 1.88:1. In one or more embodiments, the composition comprises a hydrophobic solvent in a concentration of about 27% to about 73% by weight of the composition. For e.g. about 27% to about 35% by weight, about 35% to about 40% by weight, about 40% to about 45% by weight, about 45% to about 50% by weight, about 50% to about 55% by weight, about 55% to about 60% by weight, about 60% to about 65% by weight, about 65% to about 70% by weight, and about 70% to about 73% by weight of the composition. In one or more embodiments, the composition comprises a hydrophobic solvent in a concentration of about 27% by weight of the composition or about 30% by weight, or about 35% by weight, or about 40% by weight, or about 45% by weight, or about 50% by weight, or about 55% by weight, or about 60% by weight, or about 65% by weight, or about 70% by weight, or about 73% by weight of the composition.

In some embodiments, the hydrophobic phase comprises a combination of two or more liquid oils. In some embodiments, the hydrophobic solvent comprises a combination of a mineral oil and one or additional hydrophobic solvents. In some embodiments, the hydrophobic solvent comprises a combination of a mineral oil and one or more of caprylic/capric triglycerides (MCT), octyl dodecanol (OD), soybean oil, isopropyl myristate and cyclomethicone. In certain embodiments, the composition is substantially free, essentially free or free of one or more of soybean oil, isopropyl myristate, dimethicone and cyclomethicone. In some embodiments, the ratio between the oil and polar solvent in the herein disclosed compositions is more than about 0.5:1. For example, more than about 0.8:1, or more than about 1:1. In some embodiments, the ratio between the oil and polar solvent in the herein disclosed compositions is about or more than about 1.5:1, about or more than about 2:1, about or more than about 2.5:1, about or more than about 3:1, about or more than about 3.5:1, about or more than about 4:1, about or more than about 4.5:1, about or more than about 5:1. In some embodiments, the ratio between the oil and polar solvent in the herein disclosed compositions is more than about 0.88:1, or about or more than about 1.88:1.

In certain embodiments, the hydrophobic phase comprises a petrolatum. In certain embodiments, the hydrophobic phase comprises a combination of a liquid oil and a petrolatum. In some embodiments, the hydrophobic phase comprises a combination of a mineral oil and a petrolatum; in some embodiments comprises a combination of a plant oil with a petrolatum; and in some embodiments comprises a combination of a plant and a mineral oil with a petrolatum.

### Active Pharmaceutical Agents

In various embodiments, the active pharmaceutical agent is fully or partially solubilized in the composition or in at least one phase of the composition, or is present as a solubilized form. In some embodiments, more than about 30% of the active agent is solubilized in the composition. In some embodiments, more than about 40%, more than about 50%, more than about 60%, more than about 70%, more than about 80%, more than about 85%, more than about 90%, more than about 95%, more than about 97%, more than about 98%, or more than about 99% of the active agent is solubilized in the composition. In certain embodiments, all the active ingredient is solubilized. In one or more embodiments, a portion of the active agent is suspended in the formulation. In some embodiments, the suspended active ingredient is micronized before being suspended. In one or more embodiments, the majority of the micronized particles are between about 18 microns and 1 micron, between about 15 microns and 1.5 microns, between about 13 microns and 2 microns, between about 11 microns and 2.5 microns, between about 9 microns and 3 microns, between about 8 microns and 3.5 microns, or between about 7 microns and 4 microns. In one or more embodiments, the majority of the particles are less than about 18 microns, less than about 15 microns, less than about 13 microns, less than about 11 microns, less than about 9 microns, less than about 7 microns, or less than about 5 microns. In one or more embodiments, the majority of the particles are about 18 microns, about 15 microns, about 13 microns, about 11 microns, about 10 microns, about 9 microns, about 8 microns, about 7 microns, about 6 microns, about 5 microns, about 4 microns, about 3 microns, or about 2 microns. In one or more embodiments, the majority of the particles are more than about 1.5 microns, more than about 2 microns, more than about 2.5 microns, more than about 3 microns, more than about 3.5 microns, or more than about 4 microns. The term “majority,” as used herein, refers to about 50% or more, about 60% or more, about 65% or more, about 70% or more, about 75%, more about 80% or more, about 85% or more, or about 90% or more of the total amount.

In some embodiments, the active agent is suspended in the polar phase of the composition described herein. In some embodiments, the active agent is solubilized in the polar phase. In some embodiments, the active agent is suspended in the hydrophobic phase of the composition described herein. In some embodiments, the active agent is solubilized in the hydrophobic phase. In some embodiments, part of the active agent is solubilized in the hydrophobic phase while the rest being suspended in the polar phase. In some embodiments, part of the active agent is solubilized in the polar phase while the rest being suspended in the hydrophobic phase.

In some embodiments, the active agent is present in the composition in a concentration of about 0.01% to about 10%, about 0.01% to about 8%, about 0.01% to about 6%, about 0.01% to about 4%, about 0.01% to about 3%, about 0.1% to about 10%, about 0.1% to about 8%, about 0.1% to about 6%, about 0.1% to about 4%, or about 1% to about 5% by weight of the composition. For example, the active agent is about 0.1%, about 0.5%, about 0.75%, about 1%, about 1.1%, about 1.2%, about 1.3%, about 1.4 %, about 1.5%, about 1.6 %, about 1.7%, about 1.8%, about 1.9%, about 2%, about 2.1%, about 2.2%, about 2.3%, about 2.4%, about 2.5%, about 2.6%, about 2.7%, about 2..8%, about 3%, about 4%, about 5%, about 6%, about 7%, about 8%, about 9%, about 10%, about 14%, about 18%, or about 22% by weight of the composition. In some embodiments, the active agent is about 2.33% by weight of the composition. In some embodiments, the active agent is about 2% by weight of the composition.

In one or more embodiments, the active agent is a single agent or comprises a combination of two or more active agents.

In one or more embodiments, the active agent comprises an antibacterial agent. In certain embodiments, the antibacterial active agent is a tetracycline antibiotic. In one or more embodiments, the tetracycline antibiotic is oxytetracycline, demeclocycline, doxycycline, lymecycline, meclocycline, methacycline, minocycline, rolitetracycline, chlorotetracycline, tigecycline, or a mixture of two or more thereof. In one or more embodiments, the tetracycline is minocycline or a salt thereof. In one or more embodiments, the tetracycline is minocycline hydrochloride. In one or more embodiments, the tetracycline is doxycycline or a salt thereof. In one or more embodiments, the tetracycline is doxycycline hyclate. In one or more embodiments, the tetracycline is doxycycline monohydrate. In one or more embodiments, the tetracycline antibiotic is present in a free base form, a hydrate form, a salt form, or a complex form. In one or more embodiments, the tetracycline is soluble or is partially soluble in the composition. In one or more embodiments, a part of the tetracycline is suspended in the composition. In one or more embodiments, properties or uses discovered for doxycycline or minocycline compositions can be applicable to other tetracycline antibiotic compositions.

In one or more embodiments, a composition provided herein comprises one or more active agents selected from, but not limited to, one or more of lysine, an active herbal extract, an acaricides, an age spot and keratose removing agent, an allergen, an alpha hydroxyl acid, an analgesic agent, an antiacne agent, an antiallergic agent, an antiaging agent, an antibacterial agent, an antibiotic, an antiburn agent, an anticancer agent, an antidandruff agent, an antidepressant, an antidermatitis agent, an antiedemic anent, an antifungal agent, an antihistamine, an antihelminth agent, an antihyperkeratolyte agent, an anti-infective agent, an antiinflammatory agent, an antiirritant, an antilipemic agent, an antimicrobial agent, an antimycotic agent, an antioxidant, an antiparasitic agent, an antiproliferative agent, an antipruritic agent, an antipsoriatic agent, an antirosacea agent, an antiseborrheic agent, an antiseptic agent, an antiswelling agent, an antiviral agent, an anti-wart agent, an anti-wrinkle agent, an antiyeast agents, an astringent, a beta-hydroxy acid, benzoyl peroxide, a topical cardiovascular agent, a chemotherapeutic agent, a corticosteroid, an immunogenic substance, a dicarboxylic acid, a disinfectant, a fungicide, a hair growth regulator, a haptene, a hormone, a hydroxy acid, an immunosuppressant, an immunoregulating agent, an immunomodulator, an insecticide, an insect repellent, a keratolytic agent, a lactam, a local anesthetic agent, a lubricating agent, a masking agent, a metals, a metal oxide, a mitocide, a neuropeptide, a non-steroidal anti-inflammatory agent, an oxidizing agent, a pediculicide, a peptide, a protein, a photodynamic therapy agent, a radical scavenger, a refatting agent, a retinoid, a sanative, a scabicide, a self-tanning agent, a skin protective agent, a skin whitening agent, a steroid, a steroid hormone, a vasoconstrictor, a vasodilator, a vitamin, a vitamin A, a vitamin A derivative, a vitamin B, a vitamin B derivative, a vitamin C, a vitamin C derivative, a vitamin D, a vitamin D derivative, a vitamin D analog, a vitamin F, a vitamin F derivative, a vitamin K, a vitamin K derivative, a wound healing agent and a wart remover, an androgen, an anti-hyperkeratosis agent, an estrogen, an immunostimulent, a pesticide, a progesterone, an azole, metronidazole, a sedative, a vaso active agent and mixtures of any two or more active agents.

In some embodiments, a composition provided herein comprises a tetracycline antibiotic and at least one additional active agent, for example, a tertracycline antibiotic and a retinoid, a tetracycline antibiotic and a steroid, or a tetracycline antibiotic and a retinoid and a steroid. In one or more embodiments the retinoid is adapalene or tazarotene.

In some embodiments, and as shown herein below in the examples section, a certain amount of a polar solvent is required to solubilize a certain amount of an active agent without re crystallization and precipitation. In an exemplary embodiment, 2.33% of doxycycline hyclate may be dissolved in 20% glycerin and 1.16% doxycycline hyclate may be dissolved in 10% glycerin. Thus, in one or more embodiments, the ratio between the polar solvent to active agent is about 6:1 to about 10:1. In one or more embodiments, the ratio between the polar solvent to active agent is about 8.58:1, or about 8.6:1. In one or more embodiments, the ratio between the polar solvent to active agent is about 7:1 or about 8:1 or about 9:1. in one or more embodiments, the ratio between the polar solvent to active agent is at least about 6:1, or at least about 7:1, or at least about 8:1, or at least about 9:1, or at least about 10:1. In one or more embodiments, the active agent remains stable in the composition for at least 6 months. In one or more embodiments, after storage at 5° C. for at least six months, the composition retains above about 90% of the active agent initially present in the composition, or in more embodiments above 92% or above 94% or above 95% or above 97%. In one or more embodiments, after storage at 25° C. for at least six months, the composition retains above about 90% of the active agent initially present in the composition, or in more embodiments above 92% or above 94% or above 95% In one or more embodiments, the active agent remains stable in the composition for at least 3 months at 5° C. or at 25° C. and the composition retains above about 90% of the active agent initially present in the composition, or in more embodiments above 92% or above 94% or above 95%. In one or more embodiments, the active agent remains stable in the composition for at least 2 months at 5° C. or at 25° C. and the composition retains above about 90% of the active agent initially present in the composition. In one or more embodiments, after storage at 40° C. for at least 2 months, the composition retains above about 90% of the active agent initially present in the composition.

### Surface-Active Agents

In various embodiments, the compositions of the invention are substantially free of, essentially free of, or completely free of surfactants.

Generally, surfactants are known to possess irritation potential. One way to try and reduce or minimize potential irritation and drying of the skin or mucosa due to surfactants and their repeated use, especially when formulations are to be left on the skin or mucosa rather than being washed off, is to use essentially or primarily nonionic surfactants at concentrations preferably below 5%.

Non-limiting examples of classes of non-ionic surfactants that according to the present invention include: (i) polyoxyethylene sorbitan esters (polysorbates), such as polysorbate 20, polysorbate 40, polysorbate 60 and polysorbate 80; (ii) sorbitan esters, such as sorbitan monolaurate and sorbitan monooleate; (iii) polyoxyethylene fatty acid esters, such as, PEG-8 stearate, PEG-20 stearate, PEG-40 stearate, PEG-100 stearate, PEG-150 distearate, PEG-8 laurate, PEG-10 laurate, PEG-12 laurate, PEG-20 laurate, PEG-8 oleate, PEG-9 oleate, PEG-10 oleate, PEG-12 oleate, PEG-15 oleate and PEG-20 oleate;. (iv) PEG-fatty acid diesters; (v) polyethylene glycol (PEG) ethers of fatty alcohols; (vi) glycerol esters, such as glyceryl monostearate, glyceryl monolaurate, glyceryl monopalmitate and glyceryl monooleate; (vii) PEG-fatty acid mono- and di-ester mixtures; (viii) polyethylene glycol glycerol fatty acid esters; (ix) propylene glycol fatty acid esters; (x) mono- and diglycerides; (xi) sugar esters (mono-, di- and tri-esters of sucrose with fatty acids) and (xii) PEG alkyl phenols.

In some embodiments, the surfactant is a glycerol ester. In some embodiments, the composition is substantially free, essentially free, or free of glycerol esters and/or polyglycerol esters.

In some embodiments, the surfactant is a silica having silanol groups. In some embodiments, the composition is substantially free, essentially free, or free of silica having silanol groups.

In certain embodiments, the composition is free or substantially free of an ionic surfactant. In certain embodiments, the composition is free or substantially free of a zwitterionic surfactant. In certain embodiments, the composition is free or substantially free of a non-ionic surfactant. In some embodiments, a formulation as provided herein is substantially surfactant free, i.e., comprising less than 0.5% by weight; about or less than 0.4% by weight; about or less than 0.3% by weight; about or less than 0.2% by weight; or about or less than 0.1% by weight of a surfactant. In some embodiments, a formulation as provided herein is essentially free of surfactant, i.e., comprising less than about 0.05% by weight, less than about 0.01% by weight, or less than about 0.001% by weight of a surfactant. In some embodiments, a formulated as provided herein is free of surfactant, i.e., containing no surfactant or only a trace amount of surfactant.

A surfactant (also referred as emulsifier) has three primary roles: (i) to reduce as much as possible the interfacial tension between the two liquid phases, (ii) to cover as efficiently as possible the dispersed droplets and prevent interaction with other droplets, and (iii) to desorb (opposite of absorb) from the interface as little as possible by anchoring deeply and efficiently into the dispersed phase and to dangle into the continuous phase so that the repulsive forces will be contained. In other words, once the emulsifier has been adsorbed at the interface, it can remain there with high residence to stabilize the emulsion even when the repulsive forces of the continuous and dispersed phases are maximal.

Emulsions are best stabilized when the droplets dispersed in the continuous phase are fully covered by surfactant. Only surfactants that cover the interface and reside statistically most of their time around the droplets are considered efficient emulsifiers. Fatty alcohols do not meet this criterion as they have low residence around the droplets since they can reversibly move in and out of the interface and have a tendency to desorb more than they absorb.

While the term “surface active agent” or “surfactant” is sometimes used loosely in the art and some publications and may refer to compounds that have a supportive role, such as co-surfactants, substances that cannot function as surfactants on their own are not considered to be surfactants for the purposes described herein. Thus, in the context herein, a fatty alcohol is not regarded as a surfactant, and likewise, a fatty acid is not regarded as a surfactant. In contrast, however, an ether or an ester formed from either a fatty acid or a fatty alcohol can be a surfactant. In addition, quaternary ammonium compounds and ions are regarded as surfactants in the present application. In certain circumstances, substances can be converted into a surfactant, for example, adding a base, such as, triethanolamine to a fatty acid like stearic acid. Such combinations of ingredients are regarded as surfactants even though the individual ingredients may not be considered customary surfactants on their own.

### Water

In some embodiments, the compositions provided herein are substantially waterless, essentially waterless, or free of water. In some embodiments, a composition as provided herein has a low water content.

In some embodiments the only water present is that associated with the active agent.

In some embodiments, the composition has a low water content, comprising preferably below about 5%, below about 4%, below about 3%, below about 2.5%, below about 2%, below about 1.5%, or below about 1% by weight of water.

In some embodiments, the composition is substantially free of water or substantially waterless, comprising preferably less than about 0.5% by weight; about or less than 0.4% by weight; about or less than 0.3% by weight; about or less than 0.2% by weight; or about or less than 0.1% by weight of water.

In some embodiments, the composition as provided herein is essentially water free or essentially free of water, comprising preferably less than about 0.05% by weight; about or less than 0.01% by weight; about or less than 0.005% by weight.

In some embodiments, the composition as provided herein is free of water or waterless, meaning no water. In some embodiments, the composition is essentially or substantially free of water.

It will be understood by a person of the art that the waterless solvents and substances miscible with them can be hydrophilic and can therefore contain water in an associated or unfree or absorbed form and may absorb water from the atmosphere. The ability to do reflects the hygroscopic capacity of these other ingredients. In some embodiments these composition ingredients are pretreated to reduce, remove. minimize or eliminate any residual or associated or absorbed water. Some of water appears to originate from the polar solvent, e.g. glycerin, which is known to be hygroscopic. This could be minimized or eliminated if manufacture was carried out under closed conditions, e.g., under vacuum and/or nitrogen.

### Dehydrating Agents Free

In some embodiments, a formulation as provided herein is substantially free of, essentially free of, or free of a dehydrating agent.

As used herein, the term “dehydrating agent” refers to any agent, such as a solvent that removes water from a material. The dehydrating agent may be a C₁₋₆ alkyl alcohol, ethyl acetate, or acetic anhydride. The dehydrating agent may be a monohydric alcohol. Exemplary C₁₋₆ alkyl alcohols include, but are not limited to, one or more of methanol, ethanol, n-propyl alcohol, isopropyl alcohol, n-butyl alcohol, t-butyl alcohol, 2-butanol, iso-butanol, pentanol, hexanol, and cyclohexanol.

### Anti-Oxidants

According to some embodiments, disclosed herein are compositions substantially free or essentially free or free of an antioxidant. According to other embodiments, a composition can comprise an anti-oxidant. Exemplary antioxidants include, without limitation, butylated hydroxyanisole (BHA), butylated hydroxytoluene (BHT), tertiary butyl hydroquinone, propyl gallate, α-tocopherol, sodium metabisulfite, and the like. Further exemplary antioxidants include sulfur-containing antioxidants such as sodium metabisulfite, glutathione, N-acetylcysteine, thioproline, and taurine. Further exemplary antioxidants include an antioxidant selected from the list consisting of a sulfite compound, BHT, sodium selenite, DL-alpha tocopherol, a combination of dithioerythreitol and DL-alpha tocopherol, and sodium erythorbate. Sulfurous acid salts and organic esters (referred to collectively as “sulfites”) are also effective antioxidants, such as bisulfites, pyrosulfites, metabisulfites, and sulfites.

### Active Agent Stabilizer

In one or more embodiments, the compositions are free of, essentially free of, or substantially free of divalent metal cations. In one or more embodiments, pharmaceutical compositions described herein remain stable without such cations.

In one or more embodiments a chelator may be added to the carriers and compositions in an effective amount to chelate any divalent metal cations.

In some alternative embodiments, the compositions optionally may include at least one divalent metal cation. The divalent metal cation may be, for example a magnesium, a calcium, a copper, an iron, a zinc, or a source thereof. The compositions of the invention, as provided herein, may comprise one or more divalent metal cations.

In some embodiments, the herein disclosed compositions may include a source of a divalent metal cation. As used herein the term “source of divalent metal cation” includes, for example, a salt (e.g., a salt of divalent cation). In accordance with those embodiments, the divalent cation may be provided within the composition in the form of a salt. Exemplary divalent salts include, without limitation, metal Cl₂ anhydrous, metal Cl₂, metal oxide, metal sulfate, metal salicylate, and hydrates and combinations thereof.

In some embodiments, the divalent cation, source thereof, or salt thereof may be present in the formulation at about 0.01% to about 10%, about 0.05% to about 8%, about 0.1% to about 8%, about 0.1% to about 5%, about 0.1% to about 4%, about 0.1% to about 3%, about 0.1% to about 2.5%, about 0.5% to about 8%, about 0.5% to about 5%, about 0.5% to about 4%, about 0.5% to about 3%, about 0.5% to about 2.5%, or about 0.5% to about 2% by weight of the composition. In some embodiments, the divalent cation is present in the composition less than or at about 0.001%, about 0.05%, about 0.01%, about 0.05%, about 0.1%, about 0.5%, at about 0.1%, about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, or at about 10% by weight of the composition, or at any range in between any of the amounts listed herein.

In some embodiments, the divalent metal cation (first and/or second) may be present in the formulation at a molar ratio between the divalent metal cation and the tetracycline antibiotics of about 1:10 to 10:1, about 1:8 to 8:1, 1:6 to 6:1, 1:5 to 5:1, 1:4 to 4:1, 1:3 to 3:1, 1:2 to 2:1 or 1:1.5 to 1.5:1, or at any range in between any of the amounts listed herein.

### Sugars

In some embodiments, a disclosed formulation optionally comprises a sugar such as an oligosaccharide. In some embodiments, the sugar moiety may contribute to stabilize an active agent such as a tetracycline antibiotic in part due to the formation of a structure or complex between the sugar and the active agent.

According to some embodiments, the sugar is cyclodextrin. As used herein the term “cyclodextrin” may refer to structurally related cyclic oligomaltoses, which form a new group of pharmaceutical excipients. These are torus-shaped molecules with a hydrophilic outer surface and a lipophilic central cavity. Cyclodextrins are capable of forming water-soluble inclusion complexes with a wide variety of lipophilic water-insoluble drugs by taking up a whole drug molecule, or some part of it, into the cavity. Cyclodextrin molecules are relatively large (molecular weight ranging from almost 1000 to over 1500), with a hydrated outer surface, and under normal conditions, cyclodextrin molecules will only permeate the skin barrier with considerable difficulty. It is generally believed that the cyclodextrin molecules act as true carriers by keeping lipophilic drug molecules in solution and deliver them to the skin surface where they partition from the cyclodextrin cavity into the skin. Thus, in some embodiments, cyclodextrins may act as therapeutic enhancers.

In some embodiments, cyclodextrin is selected from a gamma cyclodextrin, a hydroxy propyl-γ-cyclodextrin, and a hydroxy propyl-β- cyclodextrin. In some embodiments, the composition described herein comprises a gamma cyclodextrin.

In some embodiments, cyclodextrin is present in the formulation at about 0.1% to about 10% by weight of the formulation, about 0.1% to about 5% by weight of the formulation, about 0.1% to about 8% by weight of the formulation, about 1% to about 10% by weight of the formulation, about 2% to about 10% by weight of the formulation, about 4% to about 10% by weight of the formulation, about 3% to about 10% by weight of the formulation, or about 5% to about 10% by weight of the formulation. In some embodiments, cyclodextrin is present in the formulation at or at about 0.1%, at about 0.5%, at about 1%, at about 1.5%, at about 2%, at about 2.5%, at about 3%, about 3.5%, at about 4%, at about 4.5%, at about 5%, at about 5.5%, at about 6%, at about 6.5%, at about 7%, at about 7.5%, at about 8%, at about 8.5%, at about 9%, at about 9.5%, at about 10% by weight of the carrier, or any range between any of the amounts listed herein.

In one or more embodiments, the compositions are substantially free of, essentially free of, or free of sugars. In one or more embodiments, the compositions are substantially free of, essentially free of, or free of oligosaccharides. In one or more embodiments, the compositions are substantially free of, essentially free of, or free of cyclodextrins.

### Polymeric Agents

In some embodiments, the compositions provided herein may optionally comprise a polymeric agent. The polymeric agent may stabilize the composition and/or control drug residence in the target organ. Polymeric agents can be classified as described below. A given polymer may belong to more than one class.

In one or more embodiments, the polymeric agent is a gelling agent. A gelling agent controls the residence of a therapeutic composition in the target site of treatment by increasing the viscosity of the composition, thereby limiting the rate of its clearance from the site. Many gelling agents known in the art possess mucoadhesive properties. The gelling agent can be a natural gelling agent or a synthetic gelling agent. The gelling agent can be an inorganic gelling agent.

Exemplary gelling agents that can be used in accordance with one or more embodiments of the present invention include, but not limited to, naturally-occurring polymeric materials, such as locust bean gum, sodium alginate, sodium caseinate, egg albumin, gelatin agar, carrageenin gum, sodium alginate, xanthan gum, quince seed extract, tragacanth gum, guar gum, starch, chemically modified starches and the like, semi-synthetic polymeric materials such as cellulose ethers (e.g., hydroxyethyl cellulose, methyl cellulose, carboxymethyl cellulose, hydroxy propylmethyl cellulose), guar gum, hydroxypropyl guar gum, soluble starch, cationic celluloses, cationic guars, and the like, and synthetic polymeric materials, such as carboxyvinyl polymers, polyvinylpyrrolidone, polyvinyl alcohol, polyacrylic acid polymers, polymethacrylic acid polymers, polyvinyl acetate polymers, polyvinyl chloride polymers, polyvinylidene chloride polymers and the like. Mixtures of two or more of the above compounds are contemplated.

In one or more embodiments, the herein disclosed compositions include a hydroxypropyl cellulose (Klucel EF). In one or more embodiments, the herein disclosed compositions are substantially free, essentially free or free of a hydroxypropyl cellulose (Klucel EF).

Further exemplary gelling agents include the acrylic acid/ethyl acrylate copolymers and the carboxyvinyl polymers sold, for example, by the B.F. Goodrich Company under the trademark of Carbopol® resins. These resins consist essentially of a colloidal water-soluble polyalkenyl polyether crosslinked polymer of acrylic acid crosslinked with from 0.75% to 2% of a crosslinking agent such as polyallyl sucrose or polyallyl pentaerythritol. Examples include Carbopol® 934, Carbopol® 940, Carbopol® 950, Carbopol® 980, Carbopol® 951 and Carbopol® 981. Carbopol® 934 is a water-soluble polymer of acrylic acid crosslinked with about 1% of a polyallyl ether of sucrose having an average of about 5.8 allyl groups for each sucrose molecule.

In one or more embodiment, the composition of the present invention includes at least one polymeric agent, which is a water-soluble cellulose ether. Preferably, the water-soluble cellulose ether is selected from the group consisting of methylcellulose, hydroxypropyl cellulose, hydroxypropyl methylcellulose (Methocel), hydroxyethyl cellulose, methylhydroxyethylcellulose, methylhydroxypropylcellulose, hydroxyethylcarboxymethylcellulose, carboxymethylcellulose and carboxymethylhydroxyethylcellulose. More preferably, the water-soluble cellulose ether is selected from the group consisting of methylcellulose, hydroxypropyl cellulose and hydroxypropyl methylcellulose (Methocel). In one or more embodiments, the composition includes a combination of a water-soluble cellulose ether; and a naturally-occurring polymeric materials, selected from the group consisting of a xanthan gum, guar gum, carrageenan gum, locust bean gum and tragacanth gum.

In some embodiments, the gelling agent includes inorganic gelling agents, such as silicone dioxide (fumed silica).

Mucoadhesive/bioadhesion has been defined as the attachment of synthetic or biological macromolecules to a biological tissue. Mucoadhesive agents are a class of polymeric biomaterials that exhibit the basic characteristic of a hydrogel, i.e., swell by absorbing water and interacting by means of adhesion with the mucous that covers epithelia. Compositions of the present invention may contain a mucoadhesive macromolecule or polymer in an amount sufficient to confer bioadhesive properties. The bioadhesive macromolecule enhances the delivery of biologically active agents on or through the target surface. The mucoadhesive macromolecule may be selected from the group consisting of an acidic synthetic polymer, preferably including at least one acidic group per four repeating or monomeric subunit moieties, such as poly(acrylic)- and/or poly(methacrylic) acid (e.g., Carbopol®, Carbomer®), poly(methylvinyl ether/maleic anhydride) copolymer, a mixture thereof and copolymers; acidic synthetically modified natural polymers, such as carboxymethylcellulose (CMC); neutral synthetically modified natural polymers, such as (hydroxypropyl)methylcellulose; basic amine-bearing polymers such as chitosan; acidic polymers obtainable from natural sources, such as alginic acid, hyaluronic acid, pectin, gum tragacanth, and karaya gum; and neutral synthetic polymers, such as polyvinyl alcohol or their mixtures. An additional group of mucoadhesive polymers includes natural and chemically modified cyclodextrin, especially hydroxypropyl-β-cyclodextrin. Such polymers may be present as free acids, bases, or salts, usually in a final concentration of about 0.01% to about 0.5% by weight.

A suitable bioadhesive macromolecule is the family of acrylic acid polymers and copolymers, (e.g., Carbopol®). These polymers contain the general structure —[CH₂—CH(COOH)—]ₙ. Hyaluronic acid and other biologically-derived polymers may be used.

Exemplary bioadhesive or mucoadhesive macromolecules have a molecular weight of at least 50 kDa, or at least 300 kDa, or at least 1,000 kDa. Favored polymeric ionizable macromolecules have not less than 2 mole percent acidic groups (e.g., COOH, SO₃H) or basic groups (NH₂, NRH, NR₂), relative to the number of monomeric units. The acidic or basic groups can constitute at least 5 mole percent, or at least 10 mole percent, or at least 25, at least 50 more percent, or even up to 100 mole percent relative to the number of monomeric units of the macromolecule.

Another group of mucoadhesive agent includes inorganic gelling agents such as silicon dioxide (fumed silica), including but not limited to, AEROSIL 200 (DEGUSSA).

Many mucoadhesive agents are known in the art to also possess gelling properties.

In some embodiments, the gelling agent may be a film forming component. The film forming component may include at least one water-insoluble alkyl cellulose or hydroxyalkyl cellulose. Exemplary alkyl cellulose or hydroxyalkyl cellulose polymers include ethyl cellulose, propyl cellulose, butyl cellulose, cellulose acetate, hydroxypropyl cellulose, hydroxybutyl cellulose, and ethylhydroxyethyl cellulose, alone or in combination. In addition, a plasticizer or a cross linking agent may be used to modify the polymer’s characteristics. For example, esters such as dibutyl or diethyl phthalate, amides such as diethyldiphenyl urea, vegetable oils, fatty acids and alcohols such as oleic and myristyl acid may be used in combination with the cellulose derivative.

In one or more embodiments, the composition of the present invention includes a phase change polymer, which alters the composition behavior from fluid-like prior to administration to solid-like upon contact with the target mucosal surface. Such phase change results from external stimuli, such as changes in temperature or pH and exposure to specific ions (e.g., Ca²⁺).

Non-limiting examples of phase change polymers include poly(N-isopropylamide) and Poloxamer 407®.

The polymeric agent, if present, may be provided in an amount in the range of about 0.01% to about 5% by weight of the composition. In one or more embodiments, the polymeric agent may be present in an amount of less than about 1% w/w of the composition. In one or more embodiments, it may be present in an amount of about 1%, about 0.9%, about 0.8%, about, 0.7%, about 0.6%, about 0.5%, about 0.4%, about 0.3%, about 0.2%, about, 0.1%, or about 0.05% by weight of the composition.

In one or more embodiments, the herein disclosed compositions may include a hydroxypropyl cellulose (Klucel EF) in an amount in the range of about 0.01% to about 5% by weight of the composition. For example, about 0.1% to about 1%, or about 0.5% by weight of the composition.

In one or more embodiments, the compositions provided herein are substantially free of a polymeric agent. In one or more embodiments, the compositions provided herein are essentially free of a polymeric agent. In one or more embodiments, the compositions provided herein are free of a polymeric agent. In one or more embodiments, the compositions provided herein are substantially free, essentially free, or free of a gelling agent. In one or more embodiments, the compositions provided herein are substantially free, essentially free, or free of a film forming agent. In one or more embodiments, the compositions provided herein are substantially free, essentially free, or free of a glyceryl polyacrylate. In some embodiments, a composition has less than 5%, 4%, 3%, 2%, 1%, 0.5%, 0.4%, 0.3%, 0.2%, 0.1%, 0.05%, 0.04%, 0.03%, 0.02%, or 0.01%, by weight of the composition of a polymeric agent, a water gelling agent, and/or a film forming agent. In some embodiments, where a polymeric agent, a water gelling agent, and/or a film forming agent present at an amount of 5% by weight of the composition is not be able to gel water it is not considered water gelling agent herein.

### Penetration Enhancers

As used herein, the term “skin penetration enhancer”, also interchangeably referred herein as a “penetration enhancer,” is an organic solvent, typically soluble in both water and oil, that may facilitate the penetration of an active agent into the skin. Exemplary penetration enhancers include, but are not limited to, one or more of glycerol (glycerin), propylene glycol, hexylene glycol, diethylene glycol, propylene glycol n-alkanols, terpenes, di-terpenes, tri-terpenes, terpen-ols, limonene, terpene-ol, 1-menthol, dioxolane, ethylene glycol, hexylene glycol, other glycols, sulfoxides, such as dimethylsulfoxide (DMSO), dimethylformamide, methyl dodecyl sulfoxide, dimethylacetamide, dimethylisosorbide, monooleate of ethoxylated glycerides (with 8 to 10 ethylene oxide units), azone (1-dodecylazacycloheptan-2-one), 2-(n-nonyl)-1,3-dioxolane, esters, such as isopropyl myristate/palmitate, ethyl acetate, butyl acetate, methyl propionate, capric/caprylic triglycerides, octylmyristate, dodecyl-myristate; myristyl alcohol, lauryl alcohol, lauric acid, lauryl lactate ketones; amides, such as acetamide oleates such as triolein; various alkanoic acids such as caprylic acid; lactam compounds, such as azone; alkanols, such as dialkylamino acetates, and a mixture of two or more thereof. In some embodiments, skin penetration enhancers remain in association with an active agent during skin penetration and/or provide for increased physical and/or chemical stability of the active agent.

Polyols and hydrophobic oils may act as penetration enhancers. In some embodiments, the disclosed formulations comprising a polyol and a hydrophobic oil may optionally comprise one or more penetration enhancers that are not polyol or hydrophobic oils and supplement or enhance the penetration of an active agent.

The penetration enhancer, if present in addition to or supplementary to the polyol and hydrophobic liquid oil, may be provided in an amount in the range of about 0.01% to about 25% by weight of the composition. In one or more embodiments, it may be present in an amount of about 5% to about 15% w/w of the composition. In one or more embodiments, it may be present in an amount of about 24%, about 22%, about 20%, about 18%, about 16%, about 14%, about 12%, about 11%, about 10%, about 9%, about 8%, about 7%, about 6%, about 5%, about 4%, about 3%, about 2%, or about 1% by weight of the composition.

In one or more other embodiments, the compositions are substantially free of, essentially free of, or free of penetration enhancers that are not polyols and/or are not hydrophobic non-ester oils. In one or more other embodiments, the compositions are substantially free of, essentially free of, or free of penetration enhancers that are not glycerol and/or are not a mineral oil. In one or more other embodiments, the compositions are substantially free of, essentially free of, or free of penetration enhancers that are not propanol and are not hydrophobic non-ester oils.

### pH Adjusters

In some embodiments, the compositions may optionally comprise one or more pH adjusters or pH agents, which can include any pharmaceutically acceptable composition, compound, or agent, suitable for adjusting the pH of the presently described topical pharmaceutical compositions without negatively affecting any property thereof. The pH adjusters or pH agents may without being bound by any theory help modulate the artificial pH of the formulation and prevent or retard breakdown of active agents that that can be caused or catalyzed or encouraged, or accelerated, by small impurities present in excipients.

Suitable pH adjusters can include any pharmaceutically acceptable acid or base. In some embodiments, the pH adjustor comprises an acidic pH adjuster, such as a hydroxy acid. In some embodiments, the pH adjustor comprises a basic pH adjuster. For example, in some embodiments, the pH adjuster is an amine base. Exemplary amine bases are known in the chemical and pharmaceutical arts and include, for example, triethanolamine (i.e., Trolamine). In some embodiments, a basic pH adjuster is a hydroxide. In some embodiments, the hydroxide is in the form of a salt of an alkali or alkaline earth metal. For example, a hydroxide salt can be selected from sodium hydroxide, potassium hydroxide, and calcium hydroxide. In some embodiments, a pH adjuster is a carbonate. In some embodiments, the carbonate is in the form of a salt of an alkali or alkaline earth metal. For example, a carbonate salt can be selected from sodium carbonate, potassium carbonate, and calcium carbonate.

In some embodiments, the described pH adjusters, if present, may be present in an amount of about 0.01 % to about 1% by weight of the composition. In one or more embodiments, the pH adjusters are present in an amount of about 1%, about 0.9%, about 0.8%, about, 0.7%, about 0.6%, about 0.5%, about 0.4%, about 0.3%, about 0.2%, about 0.1%, about 0.05%, or about, 0.01% by weight of the composition.

In some embodiments, the compositions described herein are substantially free of pH adjusters or pH agents. In some embodiments the compositions are essentially free of pH adjusters or pH agents. In some embodiments, the compositions are free of pH adjusters or pH agents.

### Additional Components

In some embodiments, a composition disclosed herein includes one or more optional additional components. Such additional components include but are not limited to bulking agents, diluents, humectants, moisturisers, keratolytic agents, preservatives, pH preservatives, pH adjusters, protectants, skin penetration or permeation enhancers, solubilizers, penetration enhancers, sunscreens, sun blocking agents, and viscosity modifiers. As is known to one skilled in the art, in some instances a specific additional component may have more than one activity, function or effect.

### Foam Formulations

### Propellants

According to some embodiments, the herein disclosed compositions may be provided as foams. In accordance with those embodiments, the compositions comprise a propellant. The herein disclosed compositions, when packaged in an aerosol container and pressurized with a propellant, form a foam upon release from the container. Such foam may break upon application of shear force.

Suitable propellants include volatile hydrocarbons such as butane, propane, isobutane or mixtures thereof. In some embodiments, a hydrocarbon mixture AP-70 is used. In some embodiments, a lower pressure hydrocarbon mixture AP-46 is used. Both contain butane, propane, and isobutane although in different proportions. AP-46 is composed of about 16% w/w of propane, about 82% w/w of isobutane and about 2% w/w of propane. AP-70 is composed of about 50% w/w of propane, about 20% w/w of isobutane and about 30% w/w of propane. Hydrofluorocarbon (HFC) propellants are also suitable as propellants in the context disclosed herein. Exemplary HFC propellants include 1,1,1,2-tetrafluorethane (Dymel 134), and 1,1,1,2,3,3,3 heptafluoropropane (Dymel 227). Dimethyl ether is also useful. In some embodiments, the use of compressed gases (e.g., air, carbon dioxide, nitrous oxide, and nitrogen) is also possible.

In some embodiments, a combination of at least two propellants, selected from HFC, hydrocarbon propellants, dimethyl ether and compressed gases may be used in the formulations provided herein.

Any concentration of the propellant, which affords administrable foam is useful in accordance with the present invention. In some embodiments, in preparing the formulations all the ingredients other than propellant are combined to be 100% and the propellant is added thereafter. For example, if all the ingredients other than propellant amount to 100 grams by weight and 10 grams of propellant are added then the ratio of all ingredients other than propellant, to propellant is 100:10. Likewise, if the carrier amounts to 100 grams and 10 grams of propellant is added then the ration of carrier to propellant is 100:10. In some embodiments, all % values are provided on a weight (w/w) basis, based on the composition without propellant. In some embodiments, the propellant is present in the foam formulations between about 3% and about 28% by weight of the formulation, or between about 4% and 25% or between about 5% and about 20%, or between about 6% and 18%. In some embodiments, the propellant is present in the foam formulations at about 5% and about 15% by weight of the formulation, about 5% to about 10% by weight of the formulation, about 10% to about 15% by weight of the formulation, or about 6% to about 12% by weight of the formulation. In some embodiments, the propellant is present at about 5%, at about 6%, at about 7%, at about 8%, at about 9%, at about 10%, at about 11%, at about 12%, at about 13%, at about 14%, or at about 15% by weight of the formulation.

In some embodiments, the pressure of the propellant in the aerosol is less than about 125 psi, less than about 110 psi, less than about 100 psi, less than about 90 psi, less than about 80 psi, less than about 75 psi, less than about 70 psi, less than about 60 psi, or less than about 50 psi, less than about 40 psi, less than about 30 psi, or less than about 20 psi. In some embodiments, the pressure of the propellant in the aerosol is about 20 psi to about 70 psi, e.g., about 20 psi, about 25 psi, about 30 psi, about 35 psi, about 40 psi, about 45 psi, about 50 psi, about 55 psi, about 60 psi, about 65 psi, or about 70 psi. In some embodiments, it is between about 30 psi to about 90 psi. In some embodiments, it is between about 40 psi to about 80 psi. In some embodiments, it is present in any range between any of the amounts listed herein.

In some embodiments, the propellant can also be used to expel formulation using a bag in can system or a can in can system as will be appreciated by someone skilled in the art. In some embodiments, part of the propellant system is in the formulation and part of the propellant system is separated from the formulation. In this way it is possible to reduce the amount of propellant in the formulation but still provide good expulsion from the canister, where the foamable formulation is expelled quickly but without jetting or noise.

In some embodiments, the propellant is used in a canister with a valve suitable to forming a spray so that upon release from the canister the contents are expelled as a spray. In some other embodiments the composition is expelled as a spray that produces a fine or thin foam type coating on the skin or mucosa, for example somewhat resembling a hoar frost. As would be appreciated by one skilled in the art higher amounts of propellant are generally used to generate a spray. For example in excess of about 25%, about 30%, about 35%, about 40% about 45%, about 50%, about 55%, about 60% about 65%, about 70%, about 75%, about 80% by weight of the composition or at a range amount between any of the herein mentioned figures. In some embodiments, the propellant is about 70% to about 98% by weight of the composition, or about 25% to about 80% by weight of the composition, or about 6% to about 10% by weight of the composition.

### Foam Application

A foam formulation as provided herein is very easy to use. When applied onto the afflicted body surface of mammals, i.e., humans or animals, it is in a foam state, allowing free application without spillage. Upon further application of a mechanical force, e.g., by rubbing the composition onto the body surface, it spreads on the surface and is absorbed. In one or more embodiments, the rubbing action can be gentle and a simple rub may suffice. In one or more embodiments, the foam will collapse on the application of a mechanical or shear force. A low, mild or gentle force may be sufficient to facilitate the foams collapse. In an embodiment, most or all of the foam collapses immediately on application of a mechanical or shear force. In one or more embodiments, the foam spreads and collapses on application of a shear or mechanical force. In one or more embodiments, the absorption of the formulation is rapid. In one or more embodiments, the majority of the formulation is absorbed in less than 60 seconds, in less than 50 seconds, in less than 40 seconds, in less than 30 seconds, in less than 20 seconds, in less than 10 seconds, in less than 8 seconds, in less than 6 seconds, in less than 5 seconds, in less than 4 seconds, in less than 3 seconds, in less than 2 seconds, in less than 1 second, or about instantly. In one or more embodiments, the hydrophilic phase of the formulation is absorbed in less than 60 seconds, in less than 50 seconds, in less than 40 seconds, in less than 30 seconds, in less than 20 seconds, in less than 10 seconds, in less than 8 seconds, in less than 6 seconds, in less than 5 seconds, in less than 4 seconds, in less than 3 seconds, in less than 2 seconds, in less than 1 second, or about instantly. In illustrative embodiments, foam formulations described herein facilitate easy treatment with a combination of at least one or more the following characteristics: freely spreadable, rapidly absorbable, and low density. Other desirable characteristics include:

Uniformity: The composition should be formulated so that it is and can remain uniform or substantially so over time. This property is of particular importance when the product is intended to be a pharmaceutical product with a shelf life of about a year, or about 15 months, or about 18 months or about two years. In some embodiments, the formulation is shaken before use and is readily reforms a homogenous composition upon shaking so the composition is uniform when dispensed.

Flowability: The composition, when placed in an aerosol container and pressurized should be flowable such that it can be expelled through the canister valve. It should preferably also be shakable inside the container. These requirements create a formulation challenge, because low or non-viscous flowable and shakable compositions are prone to undergo phase separation or precipitation.

Quality: Upon release from the can, the composition should generate a foam of good or excellent quality having low density and small bubble size.

Stability/Breakability: The fine balance between stability and breakability of the foam coming out of the container is very delicate: on one hand the foam should preferably not be “quick breaking”, i.e., it should be at least short term stable upon release from the pressurized container and not break as a result of exposure to skin temperature; by short term stable is meant that upon exposure to a temperature of 36° C., it has a collapse time of 60 seconds or more than 60 seconds, or more than 90 seconds, or more than 120 seconds, or more than 150 seconds, or more than 180 seconds, or more than 120 seconds, and on the other hand, it should be “breakable”, i.e., it should spread easily, break down and absorb into the skin or membrane upon application of mild shear force.

Skin Feeling: To ensure patient compliance the skin feeling after application should be pleasant, and greasy residues should be minimal. The use of skin feeling agents can help ameliorate the presence of substantial wax in the compositions. Likewise forming soft waterless or substantially waterless gel and ointment carriers and compositions and introducing thickened silicones can aid skin feeling.

Non-irritating: The above requirements should be achieved with the awareness that formulation excipients, especially surfactants, can be irritating, and should preferably be eliminated from the composition or reduced as much as possible.

Delivery: The composition should also be designed to ensure delivery of a therapeutic agent into the target site of treatment.

In one or more embodiments, the compositions include permeation enhancers e.g., alcohols, fatty alcohols, glycols and flexible vesicles which have an advantage in penetrating into and through the skin and mucosal membranes.

Solubility: In an embodiment the therapeutic agent is soluble in the composition.

In some embodiments, the foam formulation has an acceptable shelf-life of at least one year, or at least 15 months, or at least 18 months, or at least two years at ambient temperature. One of ordinary skill in the art would appreciate that propellants tend to impair stability. Addition of propellant can dilute the formulation and upon release of the formulation the position can reverse. Dissipation of propellant from the formulation on release can increase the formulation concentration. Dilution and concentration increase can affect stability. The foam formulations herein are surprisingly stable, even in the absence of surfactants and even on dilution and concentration.

In one or more embodiments the foam formulations provided herein can provide desirable texture; form bubble structures that do not break immediately upon contact with a surface, spread easily on the treated area and allow the active agent to be absorbed on the skin or mucosa. In one or more embodiments the foams do not exhibit rapid drainage, so that the oils present in the foam do not immediately drain onto the skin.

A foam formulation as provided herein should also be free flowing, e.g., to allow it to flow through the aperture of the container and aerosol container, and create an acceptable foam.

Upon release from an aerosol container, the foam formulations provided herein form an expanded breakable foam suitable for topical administration. In some embodiments, the foam is a breakable foam that is thermally stable upon dispensing, for example, as selected by a collapse time of about 60 secs or more; and yet breaks easily upon application of shear force.

Thus, in one or more embodiments, there is provided a foam formulation that provides a good to excellent breakable foam. In some embodiments, the foam displays a collapse time of about 60 seconds or more, or of about 90 seconds or more, or of about 120 seconds or more, or of about 150 seconds or more, or of about 180 seconds or more at 36° C. In other words, the foam is thermally stable on exposure to a body surface at normal body temperature.

In some embodiments, the foam displays a collapse time of about 60 seconds or less, or of about 50 seconds or more, or of about 40 seconds or more, or of about 30 seconds or more at 36° C.

Breakable foam that is thermally stable, yet breaks under shear force or a mechanical force such as a simple or gentle rub is desirable. Shear-force breakability of the foam is clearly advantageous over thermally-induced breakability. Thermally sensitive foams start to collapse immediately upon exposure to skin temperature and, therefore, cannot be effectively applied on the hand and afterwards delivered to the afflicted area.

The collapse time of foam represents its tendency to be temperature-sensitive and its ability to be at least stable in the short term so as to allow a user sufficient time to comfortably handle and apply the foam to a target area without being rushed and or concerned that it may rapidly collapse, liquefy and or disappear. Collapse time, as an indicator of thermal sensitivity, is examined by dispensing a given quantity of foam and photographing sequentially its appearance with time during incubation at 36° C.

In one or more embodiments, the foam formulation as provided herein is thermally stable on exposure to a body surface at normal body temperature.

In one or more embodiments, the foam formulation as provided herein is thermally liable on exposure to a body surface at normal body temperature.

Foam quality can be graded as follows:

Grade E (excellent): very rich and creamy in appearance, does not show any bubble structure or shows a very fine (small) bubble structure; does not rapidly become dull; upon spreading on the skin, the foam retains the creaminess property and does not appear watery.

Grade G (good): rich and creamy in appearance, very small bubble size, “dulls” more rapidly than an excellent foam, retains creaminess upon spreading on the skin, and does not become watery.

Grade FG (fairly good): a moderate amount of creaminess noticeable, bubble structure is noticeable; upon spreading on the skin the product dulls rapidly and becomes somewhat lower in apparent viscosity.

Grade F (fair): very little creaminess noticeable, larger bubble structure than a “fairly good” foam, upon spreading on the skin it becomes thin in appearance and watery.

Grade P (poor): no creaminess noticeable, large bubble structure, and when spread on the skin it becomes very thin and watery in appearance.

Grade VP (very poor): dry foam, large very dull bubbles, difficult to spread on the skin.

Topically administrable foams are typically of quality grade E or G, when released from the aerosol container. Smaller bubbles are indicative of a more stable foam, which does not collapse spontaneously immediately upon discharge from the container. The finer foam structure looks and feels smoother, thus increasing its usability and appeal.

In some embodiments, topical application of an agent allows the agent to pass through the skin or mucosa and enter the blood system and effect delivery systemically.

As used herein, the term “easy spreading” or “easily spreads” means that the foam composition spreads on and over a skin or mucosal surface with a low or minimum of force such as a gentle rub or spreading motion of the hand. In some embodiments, a low, mild, or gentle force may be sufficient to facilitate the foams collapse and spreading. In some embodiments, the foam formulations provided herein are capable of spreading farther across the site of application as compared to gel and cream formulations. For example, a provided foam formulation provided herein spreads about twice as far as the same amount of a gel or cream formulation. In some embodiments, a provided foam formulation spreads about three times as far; or about four times as far; or about five times as far; or about six times as far; or about ten times as far as the same amount of a gel or cream formulation. Without being bound by any theory, the increased spreading of a provided foam formulation distributes the active agent to a larger surface area with a lesser quantity of the active agent relative to other formulations.

As used herein, the term “breakable foam” refers to a property of a gel or foam wherein the gel or foam is stable upon dispensing from a container, yet breaks and spreads easily upon application of shear or mechanical force, which can be mild, such as a simple rub.

As used herein, the term “thermally stable” means that the foam upon application onto a warm skin or body surface at about 35-37° C. (about 35 or about 36 or about 37° C.) does not immediately collapse. In one or more embodiments, the collapse time of the foam is more than about 30 seconds or more than about one minute or more than about two minutes, or more than about three minutes. In one or more limited embodiments, collapse time can be a little shorter than 30 seconds, but not less than about 20 seconds. In one or further or alternative embodiments, the collapse time is measured by introducing a sample of foam into an incubator at 36° C. and the collapse time of the foam is more than 30 seconds or more than about one minute or more than about two minutes, or more than about three minutes.

As used herein, the term “quick breaking” means the foam, spray or gel is not stable upon dispensing from a container and quickly breaks and collapses.

As used herein, the terms “quickly absorbed” or “readily absorbed” means that the composition enters onto and into an area of skin, mucosa or eye, often forming a thin coating on the surface.

### Exemplary Compositions and Pharmaceutical Formulations

The present disclosure provides compositions comprising a polar phase and an apolar phase, where mixing the two phases produces a physically stable homogenous composition. When provided as carriers for tetracycline antibiotics, the tetracycline antibiotics are partially or fully solubilized in the polar phase and exhibits chemical stability of the tetracycline antibiotic for a prolonged duration.

In one or more embodiments, the present invention provides homogenous two-phase formulations comprising a waxy hydrophobic phase and a polar phase. In one or more embodiments, the hydrophobic phase includes a paraffin wax and the polar phase is substantially free, essentially free or free of a diol. In one or more embodiments, the hydrophobic phase includes a paraffin wax and the polar phase includes a monohydric alcohol, and/or a triol. In one or more embodiments, the ratio between the wax and polar solvent is about or more than about 2:1. In one or more embodiments, provided herein are homogenous two-phase formulations comprising a monohydric alcohol or triol and a paraffin wax, wherein the ratio between the wax and polar solvent is about or more than about 2:1. In some embodiments, the herein disclosed compositions comprise up to about 70% by weight of the composition of a wax, and up to about 40% by weight of the composition of a polyol. In an exemplary embodiment, the composition comprises up to about 70% by weight of the composition of a paraffin wax, and up to about 40% by weight of the composition of a polyol, wherein the polyol is substantially free, essentially free or free of a diol. In an exemplary embodiment, the composition comprises about 66.67% by weight of the composition of a paraffin wax, and about 33.33% by weight of the composition of a polyol, wherein the polyol is substantially free, essentially free or free of a diol.

In one or more embodiments, the present invention provides homogenous two-phase formulations wherein the hydrophobic phase includes a wax and a hydrophobic solvent, and the polar phase includes a monohydric alcohol, and/or a triol. In one or more embodiments, the hydrophobic phase includes a hydrocarbon-based wax and a hydrocarbon-based hydrophobic solvent, and the polar phase includes a monohydric alcohol, and/or a triol. In one or more embodiments, the hydrophobic phase includes a paraffin wax and a mineral oil, and the polar phase includes a propanol. In an exemplary embodiment, the paraffin wax is about 5% to about 40% by weight of the composition, the mineral oil is about 30% to about 75% by weight of the composition and the propanol is about 5% to about 35% by weight of the composition. In yet a further exemplary embodiment, the paraffin wax is about 40%, the mineral oil is about 37% to about 40%, and the propanol is about 20% by weight of the composition. In one or more embodiments, the present invention provides homogenous two-phase formulations wherein the hydrophobic phase includes a wax and a hydrophobic solvent, and the polar phase includes a triol. In one or more embodiments, the hydrophobic phase includes a hydrocarbon-based wax and a hydrocarbon-based hydrophobic solvent, and the polar phase includes a triol. In one or more embodiments, the hydrophobic phase includes a paraffin wax and a mineral oil, and the polar phase includes a glycerin. In an exemplary embodiment, the paraffin wax is about 5% to about 40% by weight of the composition, the mineral oil is about 30% to about 50% by weight of the composition and the glycerin is about 25% to about 55% by weight of the composition. In yet a further exemplary embodiment, the paraffin wax is about 20%, the mineral oil is about 45%, and the glycerin is about 35% by weight of the composition.

In an exemplary embodiment, the paraffin wax is about 30% to about 50% by weight of the composition, the mineral oil is about 10% to about 50% by weight of the composition and the glycerin is about 10% to about 50% by weight of the composition.

In an exemplary embodiment, the paraffin wax is about 5% to about 20% by weight of the composition, the mineral oil is about 40% to about 70% by weight of the composition and the glycerin is about 10% to about 30% by weight of the composition. In one or more embodiments, the composition is introduced into a pressurized canister, which is then crimped. In one or more embodiments, a propellant is added to the composition. In one or more embodiments, upon release of the composition, an excellent foam is produced, having a collapse time of above 180 seconds at 36° C. temperature.

In an exemplary embodiment, the paraffin wax is about 15% to about 50% by weight of the composition, the mineral oil is about 10% to about 55% by weight of the composition and the glycerin is about 3% to about 20% by weight of the composition. In one or more embodiments, the composition further comprises an active agent. In some embodiments, the active agent is a tetracycline antibiotic, in one or more embodiments the active agent is a doxycycline. In one or more embodiments the doxycycline is about 0.1% to about 5% by weight of the composition.

In one or more embodiments, the present invention provides homogenous two-phase formulations wherein the hydrophobic phase includes a wax and a hydrophobic solvent, and the polar phase includes a triol. In one or more embodiments, the composition further comprises a polymeric agent. In one or more embodiments, the hydrophobic phase includes a hydrocarbon-based wax and a hydrocarbon-based hydrophobic solvent, the polar phase includes a triol and the polymeric agent comprises a cellulose. In one or more embodiments, the hydrophobic phase includes a paraffin wax and a mineral oil, the polar phase includes a glycerin and the polymeric agent includes a hydroxypropyl cellulose (Klucel EF). In an exemplary embodiment, the paraffin wax is about 30% to about 50% by weight of the composition, the mineral oil is about 30% to about 50% by weight of the composition, the glycerin is about 15% to about 30% by weight of the composition and the hydroxypropyl cellulose is about 0.01% to about 5% by weight of the composition. In yet a further exemplary embodiment, the paraffin wax is about 40%, the mineral oil is about 37%, the glycerin is about 20% and the hydroxypropyl cellulose is about 0.5% by weight of the composition.

In one or more embodiments, the present disclosure provides homogenous two-phase formulations wherein the hydrophobic phase includes a wax and a hydrophobic solvent, and the polar phase includes a triol. In one or more embodiments, the hydrophobic phase includes a hydrocarbon-based wax and a hydrophobic solvent comprising a hydrocarbon-based solvent and a non-hydrocarbon-based solvent, and the polar phase includes a triol. In one or more embodiments, the hydrophobic phase includes a paraffin wax, a mineral oil and a medium chain triglycerides (MCT oil), and the polar phase includes glycerin. In an exemplary embodiment, the paraffin wax is about 5% to about 40% by weight of the composition, the mineral oil is about 10% to about 30% by weight of the composition, the MCT oil is about 25% to about 45% by weight of the composition and the glycerin is about 20% to about 30% by weight of the composition. In yet a further exemplary embodiment, the paraffin wax is about 27%, the mineral oil is about 16% or about 21%, the NCT oil is about36% or about 31% and the glycerin is about 21% by weight of the composition.

In one or more embodiments, the present disclosure provides homogenous two-phase formulations wherein the hydrophobic phase includes a hydrocarbon-based wax and a hydrocarbon-based hydrophobic solvent, and the polar phase includes a triol. In one or more embodiments, the hydrophobic phase includes a paraffin wax and a mineral oil, and the polar phase includes glycerin. In an exemplary embodiment, the paraffin wax is about 5% to about 50% by weight of the composition, the mineral oil is about 25% to about 75% by weight of the composition and the glycerin is about 5% to about 35% by weight of the composition. In yet a further exemplary embodiment, the paraffin wax is about 40%, the mineral oil is about 37% to about 40%, and the glycerin is about 20% by weight of the composition. In an exemplary embodiment, the ratio between the wax to polar solvent is between about 0.25:1 to about 8:1 or about 0.25:1 to about 2.5:1. In an exemplary embodiment, the ratio between the wax to hydrophobic solvent is between about 0.07: 1 to about 1.22:1 or about 0.07:1 to about 1.8:1. In an exemplary embodiment, the ratio between the hydrophobic solvent to the polar solvent is between about 1.28: 1 to about 10.88:1 or about 3.63:1 to about 1.38:1.

In one or more embodiments, the present disclosure provides homogenous two-phase formulations wherein the wax is selected from a beeswax, a microcrystalline wax, an emulsifying wax, or a combination thereof, the hydrocarbon-based oil is a mineral oil, and the polar phase is glycerin. In an exemplary embodiment, the wax is about 5% to about 45%, the mineral oil is about 30% to about 75% and the glycerin is about 5% to about 35% by weight of the composition.

In one or more embodiments, the present disclosure provides homogenous two-phase formulations wherein the wax is a microcrystalline wax, a paraffin wax, or a combination thereof, the oil is selected from mineral oil, octyl dodecanol, caprylic/capric triglycerides, cyclomethicone or a combination thereof and the polar phase includes glycerin. In an exemplary embodiment, the wax is about 7% to about 60%, the one or more oils is about 30% to about 80% and the glycerin is about 10% to about 20% by weight of the composition. In an exemplary embodiment, the wax is a microcrystalline wax, the oil is a combination of mineral oil, octyl dodecanol, and caprylic/capric triglycerides, and the polar phase is glycerin. In an exemplary embodiment, the wax is about 7% to about 60%, the mineral oil is about 1.5% to about 50%, the octyl dodecanol is about 12%, the caprylic/capric triglycerides is about 20% and the glycerin is about 10% by weight of the composition.

In an exemplary embodiment, the wax is a microcrystalline wax, the oil comprises a combination of mineral oil, caprylic/capric triglycerides, and cyclomethicone and the polar phase is glycerin. In an exemplary embodiment, the microcrystalline wax is about 40%, the mineral oil is about 17.67%, the caprylic/capric triglycerides is about 15%, the cyclomethicone is about 5% and the glycerin is about 20% by weight of the composition. In yet a further exemplary embodiment, the wax is microcrystalline wax, a paraffin wax, or a combination thereof and is present in an amount of about 40%, the oil is a combination of mineral oil, caprylic/capric triglycerides and octyl dodecanol, wherein the mineral oil is about 16% to about 18%, the octyl dodecanol is about 0% to about 12%, the caprylic/capric triglycerides is about 15% to about 20% and the glycerin is about 10% to about 20% by weight of the composition.

In one or more embodiments, the present disclosure provides homogenous two phase formulations wherein the hydrophobic phase includes an isopropyl myristate, and paraffin wax and the polar phase includes a triol. In one or more embodiments, the hydrophobic phase includes a paraffin wax and isopropyl myristate, and the polar phase includes glycerin. In an exemplary embodiment, the paraffin wax is about 30% to about 50%, the isopropyl myristate is about 30% to about 50% and the glycerin is about 15% to about 25% by weight of the composition. In yet a further exemplary embodiment, the paraffin wax is about 40%, the isopropyl myristate is about 37% to about 40%, and the glycerin is about 20% by weight of the composition. In one or more embodiments, the present invention provides homogenous two-phase formulations wherein the hydrophobic phase includes a soybean oil, a dimethicone, a cyclomethicone, an isopropyl myristate, a mineral oil or a combination of any two or more thereof, and a paraffin wax and the polar phase includes a triol. In one or more embodiments, the polar phase includes glycerin. In an exemplary embodiment, the paraffin wax is about 30% to about 50%, the mineral oil, isopropyl myristate, soybean oil, dimethicone or cyclomethicone is about 17% to about 50% and the glycerin is about 15% to about 25% by weight of the composition. In yet a further exemplary embodiment, the paraffin wax is about 40%, the isopropyl myristate, or the mineral oil, or the soybean oil, or the dimethicone, or the cyclomethicone is about 17% to about 40%, and the glycerin is about 20% by weight of the composition.

In one or more embodiments, the present disclosure provides homogenous two-phase formulations wherein the hydrophobic phase includes a paraffin wax, petrolatum with or without mineral oil, and the polar phase includes glycerin. In an exemplary embodiment, the paraffin wax is about 30% to about 50%, the petrolatum with or without mineral oil is about 20% to about 40%, and the glycerin is about 15% to about 25% by weight of the composition. In an exemplary embodiment, the paraffin wax is about 40%, the petrolatum with or without mineral oil is about 35% to about 40%, and the glycerin is about 20% by weight of the composition. In an exemplary embodiment, the petrolatum is about 35% to about 40%, or when provided with mineral oil (at about 17% to about 18%), it is about 20% by weight.

In one or more embodiments, the present invention provides homogenous two-phase formulations wherein the hydrophobic phase includes a solid fatty alcohol and a liquid fatty alcohol and/or a liquid fatty acid, and the polar phase includes a triol and a diol. In one or more embodiments, the hydrophobic phase includes stearyl alcohol and oleyl alcohol or oleic acid, and the polar phase includes a glycerin and a propylene glycol. In some embodiments, the formulation further comprises a cetostearyl alcohol. In an exemplary embodiment, the stearyl alcohol is about 20% to about 40% by weight of the composition, the oleyl alcohol and/or oleic acid is about 5% to about 25% by weight of the composition, the cetostearyl alcohol is about 5% to about 10% by weight of the composition, the glycerin is about 15% to about 45% by weight of the composition and the propylene glycol is about 20% to about 30% by weight of the composition.

In one or more embodiments, the present invention provides homogenous two-phase formulations wherein the hydrophobic phase includes a solid fatty alcohol and a liquid fatty alcohol and the polar phase includes a triol and a diol. In one or more embodiments, the hydrophobic phase includes stearyl alcohol and oleyl alcohol and the polar phase includes a glycerin and a hexylene glycol. In an exemplary embodiment, the stearyl alcohol is about 30% to about 40% by weight of the composition, the oleyl alcohol is about 10% to about 20% by weight of the composition, the glycerin is about 20% to about 40% by weight of the composition and the hexylene glycol is about 15% to about 30% by weight of the composition.

In one or more embodiments, the present disclosure provides homogenous two-phase formulations wherein the hydrophobic phase includes a solid fatty alcohol and a hydrophobic solvent and the polar phase includes a triol. In one or more embodiments, the hydrophobic phase includes stearyl alcohol and mineral oil and the polar phase includes a glycerin. In an exemplary embodiment, the stearyl alcohol is about 15% to about 45% by weight of the composition, the mineral oil is about 30% to about 60% by weight of the composition and the glycerin is about 20% by weight of the composition.

In one or more embodiments, the present disclosure provides homogenous two-phase formulations wherein the hydrophobic phase includes a solid fatty alcohol, and a hydrocarbon-based oil, and the polar phase includes a triol. In an exemplary embodiment, the fatty alcohol includes about or less than about 20 or 18 carbon atoms in the backbone chain. In one or more embodiments, the fatty alcohol is selected from myristyl alcohol and stearyl alcohol, or a combination thereof. In one or more embodiments, the fatty alcohol is myristyl alcohol and/or stearyl alcohol, the oil is a mineral oil and the polar phase is glycerin. In an exemplary embodiment, the myristyl alcohol and/or stearyl alcohol is about 30% to about 50%, the mineral oil is about 35% to about 40%, and the glycerin is about 15% to about 25%. In an exemplary embodiment, the myristyl alcohol and/or stearyl alcohol is about 40%, mineral oil is about 37% to about 38%, and the glycerin is about 20% by weight of the composition.

In one or more embodiments, the present disclosure provides homogenous two phase formulations wherein the hydrophobic phase includes a wax, a hydrophobic solvent, and a thickened silicone and the polar phase includes a triol. In one or more embodiments, the hydrophobic phase includes a paraffin wax, a microcrystalline wax, a mineral oil, octyldodecanol, caprylic/capric triglycerides (MCT oil), a thickened silicone comprising one or more of cyclopentasiloxane and dimethicone crosspolymer (ST-Elastomer 10), stearoxytrimethylsilane and stearyl alcohol (Silky Wax 10), dimethicone and polysilicone-11 (Gransil DMG5), Cyclopentasiloxane (and) Polysilicone-11 (MGS-Elastomer 1100), Cyclopentasiloxane (and) Petrolatum (and) Polysilicone-11 (MGS-Elastomer 1148P) and polymethylsilsesquioxane (MGS powder 3300) and the polar phase includes a glycerin. In an exemplary embodiment, the paraffin wax is about 5% to about 20% by weight of the composition, the microcrystalline wax is about 10% to about 30% by weight of the composition, the mineral oil is about 5% to about 20% by weight of the composition, the octyldodecanol is about 5% to about 20% by weight of the composition, the MCT oil is about 10% to about 30% % by weight of the composition, the thickened silicone is about 10% to about 30% by weight of the composition and the glycerin is about 5% to about 20% by weight of the composition. In yet a further exemplary embodiment, the paraffin wax is about 10% by weight of the composition, the microcrystalline wax is about 15% or about 20% by weight of the composition, the mineral oil is about 9% or about 14% by weight of the composition, the octyldodecanol is about 10% by weight of the composition, the MCT oil is about 20% by weight of the composition, the thickened silicone is about 20% by weight of the composition and the glycerin is about 10% by weight of the composition.

In one or more embodiments, the composition is free, essentially free or substantially free of a wax.

In one or more embodiments, the present disclosure provides homogenous two-phase formulations wherein the hydrophobic phase includes a thickened silicone and a wax, and the polar phase includes a triol. In one or more embodiments, the thickened silicone comprises one or more of cyclopentasiloxane and dimethicone crosspolymer (ST-Elastomer 10), stearoxytrimethylsilane and stearyl alcohol (Silky Wax 10), dimethicone and polysilicone-11 (Gransil DMG5), Cyclopentasiloxane (and) Polysilicone-11 (MGS-Elastomer 1100), Cyclopentasiloxane (and) Petrolatum (and) Polysilicone-11 (MGS-Elastomer 1148P) and polymethylsilsesquioxane (MGS powder 3300); the triol comprises glycerin and the wax comprises paraffin wax, microcrystalline wax or mixtures thereof. In an exemplary embodiment, the dimethicone and polysilicone-11 (Gransil DMG5), cyclopentasiloxane and dimethicone crosspolymer (ST-Elastomer 10), or mixtures thereof is about 60% to about 80% by weight of the composition; the paraffin wax, microcrystalline wax or mixtures thereof is about 5% to about 35% by weight of the composition and the glycerin is about 10% by weight of the composition.

In some embodiments, the herein disclosed compositions comprise:
up to about 40% by weight of the composition of a polyol;
about 5% to about 45% by weight of the composition of a wax; and
about 15% to about 75% by weight of the composition of a hydrophobic solvent.

In some embodiments, the herein disclosed composition comprises:
about 20% by weight of the composition of a polyol;
about 20% to about 40% by weight of the composition of a wax; and
about 40% to about 60% by weight of the composition of a hydrophobic solvent.

In some embodiments, the herein disclosed composition comprises:
up to about 25% by weight of the composition of a polyol;
about 35% to about 45% by weight of the composition of a wax; and
about 30% to about 45% by weight of the composition of a hydrophobic solvent.

In some embodiments, the herein disclosed composition comprises:
up to about 25% by weight of the composition of a polyol;
about 5% to about 15% by weight of the composition of a wax; and
about 60% to about 75% by weight of the composition of a hydrophobic solvent.

In one or more embodiments, the wax is a paraffin wax 57-60, the hydrophobic liquid substance is a mineral oil or a mineral oil and a medium chain triglycerides oil, and the polyol is glycerin.

In some embodiments, the herein disclosed composition comprises:
about 15% to about 65% by weight of the composition of a polyol;
about 20% to about 45% by weight of the composition of a wax; and
about 10% to about 55% by weight of the composition of a hydrophobic liquid solvent.

In some embodiments, the herein disclosed composition comprises:
about 15% to about 45% by weight of the composition of a polyol;
about 20% to about 40% by weight of the composition of a wax; and
about 10% to about 30% by weight of the composition of a hydrophobic liquid solvent.

In one or more embodiments, the polyol is selected from the group consisting of: glycerin, propylene glycol, hexylene glycol, and a mixture thereof, the wax is stearyl alcohol, and wherein the hydrophobic solvent is oleic acid, oleyl alcohol or a combination thereof.

In some embodiments, the herein disclosed composition comprises:
about 15% to about 25% by weight of the composition of a polyol;
about 25% to about 45% by weight of the composition of a wax; and
about 30% to about 55% by weight of the composition of a hydrophobic liquid solvent.

In one or more embodiments, the polyol is glycerin, the fatty alcohol is stearyl alcohol and the hydrophobic liquid solvent is a mineral oil.

In one or more of the embodiments described herein, the composition further comprises an active agent. In some embodiments the active agent is solubilized in the polar solvent. In some embodiments, the active agent is a tetracycline antibiotic. In one or more embodiments the active agent is a doxycycline.

### Methods of Use

Provided herein are methods for treating a disorder in a subject, the method comprising topically administering to a subject in need thereof a therapeutically effective amount of a formulation provided herein.

In some embodiments, provided herein are methods for treating a condition, disease or disorder in a subject. Exemplary conditions, diseases or disorders include, but are not limited to, one or more of dermatological pain, dermatological inflammation, acne, acne vulgaris, inflammatory acne, non-inflammatory acne, acne fulminans, nodular papulopustular acne, acne conglobata, dermatitis, bacterial skin infections, fungal skin infections, viral skin infections, parasitic skin infections, skin neoplasia, skin neoplasms, pruritis, cellulitis, acute lymphangitis, lymphadenitis, erysipelas, cutaneous abscesses, necrotizing subcutaneous infections, scalded skin syndrome, folliculitis, furuncles, hidradenitis suppurativa, carbuncles, paronychial infections, rashes, erythrasma, impetigo, ecthyma, yeast skin infections, warts, molluscum contagiosum, trauma or injury to the skin, post-operative or post-surgical skin conditions, scabies, pediculosis, creeping eruption, eczemas, psoriasis, pityriasis rosea, lichen planus, pityriasis rubra pilaris, edematous, erythema multiforme, erythema nodosum, granuloma annulare, epidermal necrolysis, sunburn, photosensitivity, pemphigus, bullous pemphigoid, dermatitis herpetiformis, keratosis pilaris, callouses, corns, ichthyosis, skin ulcers, ischemic necrosis, miliaria, hyperhidrosis, moles, Kaposi’s sarcoma, melanoma, malignant melanoma, basal cell carcinoma, squamous cell carcinoma, poison ivy, poison oak, contact dermatitis, atopic dermatitis, rosacea, purpura, moniliasis, candidiasis, baldness, alopecia, Behcet’s syndrome, cholesteatoma, Dercum disease, ectodermal dysplasia, gustatory sweating, nail patella syndrome, lupus, hives, hair loss, Hailey-Hailey disease, chemical or thermal skin burns, scleroderma, aging skin, wrinkles, sun spots, necrotizing fasciitis, necrotizing myositis, gangrene, scarring, and vitiligo; and wherein the active agent is suitable for treating said disorderm or is selected from the group consisting of chlamydia infection, gonorrhea infection, herpes, human papillomavirus (HPV), genital warts, bacterial vaginosis, candidiasis, chancroid, granuloma Inguinale, lymphogranuloma venereum, mucopurulent cervicitis (MPC), molluscum contagiosum, nongonococcal urethritis (NGU), trichomoniasis, vulvar disorders, vulvodynia, vulvar pain, yeast infection, vulvar dystrophy, vulvar intraepithelial neoplasia (VIN), contact dermatitis, pelvic inflammation, endometritis, salpingitis, oophoritis, genital cancer, cancer of the cervix, cancer of the vulva, cancer of the vagina, vaginal dryness, dyspareunia, anal and rectal disease, anal abscess/fistula, anal cancer, anal fissure, anal warts, hemorrhoids, anal itch, pruritus ani, fecal incontinence, constipation, polyps of the colon and rectum; and wherein the active agent is suitable for treating said disorder.

In some embodiments, the present invention provides the use of the herein disclosed topical formulations in the manufacture of a medicament for or having an activity for the treatment, reduction or amelioration of a condition, a disease or disorder in a subject.

In some embodiments, provided herein is the use of a topical formulation to treat, ameliorate, reduce a condition, a disease or disorder in a subject by topically administering the formulation to the subject in need thereof, wherein the formulation can be applied to skin, to a mucosa, to a body cavity surface, to hair or to a nail surface.

### Methods of Application

In some embodiments, provided herein are methods of application of one or more formulations described herein on a skin and/or mucosa and/or body cavity and/or hair and/or hair follicle and/or nail surface. In one or more embodiments, the surface or a part thereof or an adjacent area is infected (hereinafter “target surface”). In one or more embodiments, the infection is microbial. In one or more embodiments, the infection is a fungal, or a yeast, or a bacterial, or a viral infection or any two or more thereof.

The herein disclosed compositions may present the form of a soft wax, an oleaginous composition, an ointment, a gel, or a foam.

In one or more embodiments, the method involves releasing the composition from a tube or canister or applicator, directly onto the surface. In one or more embodiments, the method then involves spreading the composition onto the surface using a simple mechanical force, such as a gentle rub across the target area or surface.

In one or more embodiments, the method involves releasing the composition from a canister or aerosol directly or by means of an applicator attached to the aerosol onto the surface. In one or more embodiments, the method then involves spreading the composition onto the surface. In one or more embodiments, the spread composition is absorbed. In one or more embodiments, the composition is released onto an intermediate surface such as the fingers or hand before being transferred and applied to a target surface.

The provided formulations can be topically administered. A sufficient amount of the formulation can be collapsed and spread by application of a mechanical or shear force to the composition and gentle rubbing onto the affected area and surrounding skin, for example, in an amount sufficient to cover an affected area plus a margin of healthy skin or tissue surrounding the affected area, for example, a margin of about 0.5 inches. In other embodiments of the invention there are provided formulations can be applied not only to the affected area and the surrounding skin but also to larger amounts of healthy areas, in order to prevent proliferation of the bacterial, viral or fungal infection.

The formulations provided herein can be applied to any body surface, including for example, skin surface (e.g., glabrous skin), scalp, eyebrows, eyelashes, bearded areas, nail surface, nail bed, nail matrix, and nail fold areas. In one or more embodiments, the formulations can be applied to a body cavity and a cavity surface. In one or more embodiments the formulations can be applied to an area of hairy skin, e.g., on the scalp, on the face, on the torso, on an arm and on a leg and can be applied to large areas including the entire scalp.

In some embodiments, the provided formulations can be applied in a single, one-time application, once a week, once a bi-week, once a month, from one to four times daily, or from one to three times daily, for a period of time sufficient to alleviate symptoms or clear any infection. For example, for a period of time of one week, from 1 to 12 weeks or more, from 1 to 10 weeks, from 1 to 8 weeks, from 2 to 12 weeks, from 2 to 10 weeks, from 2 to 8 weeks, from 2 to 6 weeks, from 2 to 4 weeks, from 4 to 12 weeks, from 4 to 10 weeks, from 4 to 8 weeks, from 4 to 6 weeks. The formulations provided herein can be administered, for example, at a frequency of once per day, twice per day, or three times per day. The formulations provided herein can be topically administered once per day for a period of time from 1 week to 8 weeks, from 1 week to 4 weeks, for 1 week, for 2 weeks, for 3 weeks, for 4 weeks, for 5 weeks, for 6 weeks, for 7 weeks, or for 8 weeks.

In some embodiments, after an initial treatment period of a few days of daily application, the formulations could be used less frequently than daily due to a degree of accumulation of the API in the targeted skin structures (hair follicles and shafts and the epidermis extending between the hair openings).

The provided formulations can be applied in a therapeutically effective amount, for example, an amount sufficient to cover an affected area plus a margin of healthy skin or tissue surrounding the affected area, for example, a margin of about 0.5 inches.

In some embodiments, a formulation is applied at a frequency of from one to four times daily, including for example, once daily, twice daily, three times daily, or four times daily, one a daily or weekly basis, or on a monthly or every other month schedule, for a period of time sufficient to alleviate symptoms or clear the fungal infection. For example, for a period of time from 1 to 52 weeks.

### Processes of Manufacturing

In some embodiments, provided herein are processes for formulating a topical carrier formulation comprising the steps of:
a) admixing a wax (e.g., paraffin wax 51-53, paraffin wax 57-60,) or a combination thereof with a hydrophobic liquid solvent such as an oil (e.g., mineral oil or medium-chain triglycerides) or a liquid fatty alcohol or liquid fatty acid (e.g., oleyl alcohol or oleic acid) and optionally other hydrophobic ingredients if present e.g. a solid fatty alcohol or solid fatty acid (e.g. stearyl alcohol or stearic acid respectively) while heating to about 60° C. to complete dissolution; if wax with a higher melting point is used then the formulation is heated to or above the melting point of that wax so the wax phase is dissolved;
b) heating a polar solvent (e.g., glycerin or propylene glycol or propanol or combinations thereof) to about 60° C. in a separate second vessel;
c) adding the polar phase to the hydrophobic phase while vigorously mixing at 60° C. -65° C.;
d) allowing the composition to cool down to room temperature while vigorously mixing; and optionally
e) filling the composition to an appropriate tube or container (e.g., an aerosol canister).

In some embodiments, provided herein are processes for formulating a topical formulation comprising an API, wherein the API is solubilized or partially solubilized in the polar phase, the processes comprise the steps of:
a) admixing a wax or a combination of waxes (e.g., paraffin wax 51-53 or paraffin wax 57-60 or a combination thereof) with a hydrophobic liquid solvent such as an oil (e.g., mineral oil or medium-chain triglycerides) or a liquid fatty alcohol or liquid fatty acid (e.g., oleyl alcohol or oleic acid) and optionally other hydrophobic ingredients if present e.g. a solid fatty alcohol or solid fatty acid (e.g. stearyl alcohol or stearic acid respectively) while heating to about 60° C. to complete dissolution; if wax with a higher melting point is used then the formulation is heated to or above the melting point of that wax so the wax phase is dissolved;
b) admixing in a separate second vessel a tetracycline with a polar solvent (e.g., glycerin or propylene glycol or propanol or combinations thereof) while heating to about 35° C. to complete dissolution;
c) heating the admixture of the second vessel to about 60° C.;
d) optionally heating the polar phase to about 60° C.;
e) adding the polar phase to the hydrophobic phase while vigorously mixing at 60° C. -65° C.;
f) allowing the composition to cool down to room temperature while vigorously mixing; and optionally
g) filling the composition to an appropriate tube or container, optionally an aerosol canister.

In some embodiments, provided herein are processes for formulating a topical formulation comprising an API, wherein the API is partially solubilized and partly suspended, the processes comprise the steps as aforesaid save that if an excess of API is added it can be added to one or both of the phases, before they are admixed or after they are admixed.

In some embodiments, the temperature of the heating and combining of the phase can be adjusted to accommodate the temperature sensitivity and/or melting points of the API and/or other ingredients such as waxes.

In some embodiments, provided herein is a method of stabilizing an active agent in glycerol. In some embodiments, provided herein is a method of stabilizing an active agent in glycerol in a metal salt free, anti-oxidant free composition. In some embodiments, described herein is a method of stabilizing an active agent in a two phase homogenous composition In some embodiments, the concentration of the active agent in glycerol is from about 0.1% to about 25%, or about 0.5% to about 10%, or about 1% to about 15%, or about 1% to about 20% or about 5% to about 15%, or about 1% to about 25%, and the concentration of active agent in the total composition is from about 0.1 to about 10%, or about 0.1% to about 8%, or about 0.1% to about 5%, or about 1% to about 5%.

In some embodiments, provided herein is a method of stabilizing a tetacycline in glycerol. In some embodiments, provided herein is a method of stabilizing a tetracycline in glycerol in a metal salt free, anti-oxidant free composition. In some embodiments, provided herein is a method of stabilizing a doxycycline in glycerol. In some embodiments, provided herein is a method of stabilizing a doxycycline in glycerol in a metal salt free, anti-oxidant free composition.

In some embodiments, described herein is a method of stabilizing an active agent in a two phase homogenous composition comprises admixing a wax or a combination of waxes with a) a hydrophobic liquid solvent comprising an oil, a liquid fatty alcohol, a liquid fatty acid or mixtures of any two or more thereof, b) a thickened silicone, or both while heating to about 60° C. to complete dissolution; admixing in a separate vessel an active agent with a polar solvent comprising glycerin, propylene glycol, propanol or combinations thereof while heating to about 35° C. to complete dissolution; heating the admixture of the active agent and polar phase to about 60° C.; adding the polar phase to the hydrophobic phase while vigorously mixing at 60° C.-65° C.; allowing the composition to cool down to room temperature while vigorously mixing; and optionally, filling the composition to an appropriate tube or container, optionally an aerosol canister.

In some embodiments, the process results in a stable tetracycline formulation suitable for the delivery of an active agent to, or through the skin.

According to some embodiments, and if present, the method includes adding either one or more of a penetration enhancer, an anti-oxidant, a polymer or any other suitable component.

According to some embodiments, when a foam is provided as a dosage form, the method further includes a step of filling the prepared composition into an aerosol canister, crimping and adding a propellant.

As detailed in the figures and examples below, the provided formulations have properties that contribute to forming quality solution, oleaginous, gel, ointment, foam or spray compositions suitable for the delivery of an active agent to or through the skin.

In some embodiments, the formulations provided herein exhibit stable color over prolonged time.

In some embodiments, the formulations provided herein have been shown to provide a pleasant skin feeling without causing skin itching, burning and/or stinging.

The provided formulations may lead to a better patient compliance. In some embodiments, the formulations are thermally stable, apply easily to the skin and other surfaces, provide a pleasant and smooth skin feeling after application, do not cause skin irritation, and are readily absorbed into the skin.

### EXAMPLES

The invention will be described in detail by way of specific examples. The following examples are offered for illustrative purposes, and are not intended to limit the invention in any manner. Those of skill in the art will readily recognize a variety of non-critical parameters, which can be changed or modified to yield essentially the same results.

In one or more embodiments, the amounts in the examples should be read with the prefix “about,” even if not explicitly stated.

In some embodiments, stability of the herein disclosed formulations is evaluated according to at least one or more of the following parameters: levels of epi-4 degradant, levels of epi-6 degradant, color evaluation, and levels of the active pharmaceutical ingredient (API), i.e., API assay (w/w %).

In some embodiments, the formulation, incubated for 6 months at 40° C. (equivalent to 2 years at 25° C.), should present not less than 90% API assay (i.e., no more than 10% API degradants of all types in total) and/or no more than 7% of 4-epi degradant.

### Materials

Exemplary possible ingredients used and/or suitable for the production of the compositions disclosed herein are presented at table 1. Equivalent and in some cases similar materials of pharmaceutical grade from other manufacturers can also be used.

TABLE 1A

Exemplary possible ingredients used and/or suitable for the production of the herein disclosed formulations

| | | | |
|---|---|---|---|
| Chemical Name | Function | Commercial Name | Supplier |
| Doxycycline Hyclate (DOX) | API | Doxycycline Hyclate | Hovione farmaciencia SA |
| Minocycline HCL | API | Minocycline HCl micronized | Hovione farmaciencia SA |
| Oleyl alcohol | Carrier | HD-Euthanol V PH | Cognis |
| Oleic acid | Carrier | Oleic Acid | Fluka |
| Mineral oil heavy | Carrier | Pionier 2071 P | Hansen & Rosenthal |
| Medium chain triglycerides (MCT) | Carrier | Crodamol DA-LQ | Croda |
| Soybean oil | Carrier | Soyban oil | Henry lamotte GmbH |
| Dimethicone | Carrier | Silicone oil, viscosity 350 cts (25° C.) | Sigma Aldrich |
| Petrolatum | Carrier | Pionier 3476 | Hansen & Rosenthal |
| Isostearyl alcohol | Carrier | Prisorine 3515 | Croda |
| Octyl dodecanol | Carrier | Kollicream OD | BASF |
| Cyclomethicone | Carrier | ST-cyclomethicone 5-NF | Dow coming |
| Hydrogenated castor oil | Viscosity agent | Kolliwax Hco | BASF |
| Emulsifying wax | Viscosity agent | Polawax NF—PA—(RB) | Croda |
| Microcrystalline wax | Viscosity agent | White Microcrystalline wax | Strahl & Pitch |
| Hydroxypropyl cellulose (Klucel EF) | Gelling agent | Hydroxypropyl cellulose | Sigma Aldrich |
| Paraffin 51-53 | Viscosity agent | Paraffin 51-53 | Merck |
| Paraffin wax 57-60 | Viscosity agent | Paraffin wax 57-60 | Merck |
| Cetostearyl alcohol | Viscosity agent | Kolliwax CSA 50 | BASF |
| Stearyl alcohol | Viscosity agent | Koliwx SA | BASF |
| Myristyl alcohol | Viscosity agent | Kolliwax MA | BASF |
| Behenyl alcohol | Viscosity agent | Lanette 22 | BASF |
| Isopropyl myristate (IPM) | Carrier | Isopropyl myristate | Merck |
| Hexylene glycol | Polar solvent | Hexylene glycol 99% | Sigma Aldrich |
| Transcutol | Polar solvent | Transcutol P | Gattefosse |
| Isopropanol | Polar solvent | 2-propanol | J.T. Baker |
| Dimethyl isosorbide | Polar solvent | Dimethyl isosorbide | Sricam Laboratories |
| Ethanol | Polar solvent | Ethanol | Biolab |
| Propylene carbonate | Polar solvent | Propylene carbonate | Sigma |
| Propylene glycol | Polar solvent | Kollisolv PG | BASF |
| Glycerin | Polar solvent | Palmera G995E | KLK Oleo |
| 1,3 propane-di-ol | Polar solvent | Zemea USP-NF propanediol | DuPont |
| Diisopropyl adipate | Polar solvent | Crodamol DA-LQ-(RB) | Croda |
| Dimethyl sulfoxide | Polar solvent | Dimethyl sulfoxide | Bio-lab |
| Polysilicone-11 and Cyclopentasiloxane | Carrier / Viscosity agent | MGS-Elastomer 1100 | Grant Industries |
| Cyclopentasiloxane (and) Petrolatum (and) Polysilicone-11 | Carrier / Viscosity agent | MGS-Elastomer 1148P | Grant Industries |
| Polymethylsilsesquixane | Viscosity agent | MGS powder 3300 | Grant Industries |
| dimethicone and polysilicone-11 | Carrier / Viscosity agent | Gransil DMG-5 | Grant Industries |
| Emulsifying wax, NF | Viscosity agent | Emulsifying wax, NF | spectrum |

### Tests

By way of non-limiting examples, the objectives of characterization tests are briefly set out below as would be appreciated by a person of the art.

### Chemical Stability

### α. Active Pharmaceutical Ingredient (API) and API Degradants Assessment

The amount of active pharmaceutical ingredient (API) and API degradants (e.g., 4-epi and 6-epi) present in the formulations is analyzed using Ultra-Performance Liquid Chromatography (UPLC). The total quantity of API (i.e., DOX) in the sample is designated herein as “DOX assay” and is presented as percentages of label claim. The “DOX” assay’ percentages are calculated based on the quantity detected by UPLC relative to the weight of doxycycline base added per sample and multiplied by 100%. When the data is below a detection level (i.e., less than 0.1%), it is designated as “Not Detected” (ND). When data is absent, it is designated as “Non-Available” (NA). Analysis is carried out at time zero and at appropriate time intervals thereafter. The samples are stored in sealed glass vials or aluminum canisters, under inert atmosphere (nitrogen gas) and are stored in controlled temperature incubators at 5° C., 25° C., 40° C., 50° C., 60° C., or 70° C. At selected time intervals, analysis of samples is performed. A photograph is taken (for color score record) and the amount of active agent and/or degradants content in the sample is measured by the UPLC methodology. For Doxycycline (DOX) determination, the YMC-Triart C18, 150 × 2.0 mm ID 1.9 um column is utilised.

### b. Color Change Assessment

Color change is assessed by visual inspection when the formulation which comprises a tetracycline changed color from its original appearance (pale yellow or light yellow). A color scoring is made on a scale ranging from 0 to 3 as follows: 0= light/pale yellow, 1= yellow, 2= dark yellow to light brown, and 3= brown to black.

### Microscope and Particle Size

The light microscope enables observing and measuring particles from few millimeters down to one micron. Light microscope is limited by the visible light wavelength and therefore is useful to measuring size of particles above 800 nanometers and practically from 1 micron (1,000 nanometers). The general appearance of the formulations tested herein is assessed using the light microscope.

### Phase Separation

Phase separation of formulations is analyzed by visual inspection and photographs may be taken. Samples are left at rest (e.g. at room temperature) for certain time period (e.g. at T0, or after leaving a sample at rest for 24 hours or for 72 hours or longer) or are subjected to centrifugation (e.g. at 1000 or 3000 rpm for say 1, 5 or 10 minutes or other period) and then inspected.

### Grainy Feeling/Presence of Grains

Grainy feeling of formulations is conducted via a feel inspection. A spreadable amount of a composition (e.g. ointment) is applied onto the skin protected by examination nitrile gloves (e.g. palm of hand) and presence or absence of a grainy sensation is qualitatively recorded according to feel.

### Karl Fischer Assay

Karl Fisher assay is used to determine the water content of compositions. Samples are first diluted with Combisolvent Oil, and then titrated with Combititrant 2 reagent (Merck) using a Titrino-795 titrator from Metrohm AG.

### Viscosity

Viscosity is measured with a Brookfield (e.g. LVDV-II + PRO) with a spindle at ambient temperature and 10, 5 and 1 RPM. Viscosity is usually measured at 10 RPM with spindle SC4-25. The spindle type may be adjusted so that the torque value during measurement remains in the range 10-100%.

### Foam

Foam collapse time is a measure or indication of thermal stability and is examined by dispensing a given quantity of foam and photographing sequentially its appearance with time during incubation at 36° C. The collapse time result is defined as the time when the foam height reaches 50% of its initial height. If the foam has not yet reached 50% of its initial height after 180 seconds then the collapse time is recorded as being >180 seconds. A further factor is expansion time. Some foams expand very rapidly whilst others expand quite slowly. Another factor is drainage. Formulation components that can slow drainage of fluid from the foam will result in a longer collapse time.

By way of illustration, one foam may remain at 100% of its initial height for three minutes, a second foam may reach 90% of its initial height after three minutes, a third foam may reach 70% of its initial height after three minutes, and a fourth foam may reach 51% of its initial height after three minutes, nevertheless in each of these four cases the collapse time is recorded as >180 seconds since for practical purposes for easy application by a patient to a target the majority of the foams remain intact for more than 180 seconds. If the foam for example reaches 50% of its original height after, e.g., 100 seconds it would be recorded as having a collapse time of 100 seconds. It is useful for evaluating foam products, which maintain structural stability at skin temperature for at least 1 minute. Foams which are structurally stable on the skin for at least one minute are termed “short term stable” carriers or foams.

Alternatively, a Simple Collapse Time can be assessed by placing a foam sample on the warm fingers of a volunteer and measuring the time it takes to melt on the fingers.

### Foam Density

Foam density is evaluated by dispensing the product into vessels (including dishes or tubes) of a known volume and weight. Replicate measurements of the mass of foam filling the vessels are made and the density is calculated. The foam canister with its contents are allowed to reach room temperature. The canisters are shaken to mix the contents and the first portion of the contents is discarded to waste. Then foam is dispensed into a pre-weighed tube, filling it until excess is extruded. Immediately, excess foam is removed (level off) at both ends and the filled tube is weighed on the weighing balance. The actual foam density is calculated by reducing the weight of the tube from the weight of the foam-filled tube.

### Foam Shakability

Foam shakability represents the degree to which the user is able to feel/hear the presence of the liquid contents when the filled pressurized canister is shaken. Shaking is done with normal to mild force without vigorous shaking or excessive force. When the user cannot sense the motion of the contents during shaking the product may be considered to be non-shakable. This property may be of particular importance in cases where shaking is required for affecting proper dispersion of the contents.

Table of Shakability scoring

| | |
|---|---|
| Good shakability (conforms to required quality specification) | 2 |
| Moderate shakability (conforms to required quality specification) | 1 |
| Not shakable (fails to meet required quality specification) but may still be flowable and allow foam formation of quality | 0 |
| Is substantially not able to pass through valve | Block |

### Canisters Filling and Crimping

Each aerosol canister is filled with the pre-foam formulation (“PFF”, i.e., foamable carrier) and crimped with valve using vacuum crimping machine. The process of applying a vacuum will cause most of the oxygen present to be eliminated. Addition of hydrocarbon propellant may, without being bound by any theory, further help to reduce the likelihood of any remaining oxygen reacting with the active ingredient. It may do so, without being bound by any theory, by one or more of dissolving in, to the extent present, the oil or hydrophobic phase of the formulation, by dissolving to a very limited extent in the aqueous phase, by competing with some oxygen from the formulation, by diluting out any oxygen, by a tendency of oxygen to occupy the dead space, and by oxygen occupying part of the space created by the vacuum being the unfilled volume of the canister or that remaining oxygen is rendered substantially ineffective in the formulation.

### Pressurizing & Propellant Filling

Pressurizing is carried out using a hydrocarbon gas or gas mixture. Canisters are filled either manually or with an automatic propellant filler. Canister are well shaken immediately thereafter.

### Solubility Test

Saturated solutions of doxycycline hyclate are prepared by stirring doxycycline in approximately 20 g of solvents for over 48 hours at room temperature. By the end of stirring, all samples have solids in equilibrium with liquids. The samples are filtered and subjected to quantification using UPLC.

### Skin Permeation Assay

Penetration is tested using the Franz cell in-vitro diffusion system. This system is commonly used to test the delivery of drugs through the skin from semisolid topical dosage forms. Assessment of skin penetration and permeation of the tested formulations is carried out using porcine ear skin (thickness of about 1 mm) since pig skin shows similar permeation characteristics to human skin. Vertical Franz diffusion cells (PermeGear, 1.77 cm² area, 14 ml receptor fluid) are used in the experiment. Skin samples are placed within the cells and are then subjected to incubation with the tested formulation for 24 hours at about 36° C. Analysis of doxycycline skin permeation is conducted with liquid chromatography following extraction of the tested formulation/active agent.

### Compatibility Test

Compatibility tests were performed as described in US8,343,945 and US8,945,516, which are incorporated here by reference with respect to the excipients and their compatibility described herein. Alternatively, for each excipient a mixture containing 5% active agent (e.g. minocycline HCL) in the excipient was exposed to 60° C. for 1 week and 2 weeks. The mixture was assessed for active agent degradation and optionally for color change.

### Method of Manufacture of the Herein Disclosed Formulations

Hydrophobic phase- in a first vessel a wax (e.g., paraffin wax 51-53 or paraffin wax 57-60) or saturated fatty acid or saturated fatty alcohol (e.g., stearyl alcohol) or a combination thereof is mixed with an oil (e.g., mineral oil or medium-chain triglycerides) or an unsaturated fatty acid (e.g., oleic acid) or unsaturated fatty alcohol (e.g. oleyl alcohol), or a mixture thereof while heating to 60° C. - 65° C. to complete dissolution. Higher temperatures may be used in order to account for high melting point waxes requirements to obtain full dissolution.

Polar phase- in a separate second vessel an active agent to be dissolved in the polar phase (e.g. doxycycline) if present is mixed with a polar solvent (e.g., propylene glycol or glycerin or a combination thereof) while heating to 35° C. -40° C. to complete dissolution. Then the solution is further heated to the same temperature as the hydrophobic phase.

The polar phase is then added to the hydrophobic phase while vigorously mixing. The composition is then allowed to cool down to 20° C.-25° C. while vigorously mixing. The cooled composition is then filled into appropriate tubes or containers depending on whether the composition is intended to be e.g. a flowable gel or ointment, or a foamable composition.

As will be appreciated by one skilled in the art for compositions with additional ingredients such as polymeric agents, aprotic polar solvents and hydrophilic penetration enhancers they are mixed with the polar solvent with heating. For hydrophobic additional ingredients such as silicone thickeners they are added into the hydrophobic phase while heating.

### Adjustment to 100%

Formulations in some cases did not add up to 100%, e.g., since API was omitted. To facilitate easy comparison between formulations, such formulations were adjusted to amount in a total of 100%. Formulations adjusted accordingly are herein below marked with an ^{#}.

### Example 1 - Formulation Comprising Paraffin Wax and a Polar Solvent With or Without Mineral Oil

The ability to form homogeneous formulations comprising a polar solvent, namely, alcohol or polyol, and a wax, without any emulsifier or amphiphilic compound, without a fatty acid or fatty alcohol, and without an oil, was assessed. Formulations comprising paraffin wax 51-53, and a compound that contains one, two, or three hydroxy groups in its molecular structure (2-propanol, propylene glycol, and glycerin, respectively), were prepared and evaluated. The paraffin wax is hydrophobic and the monohydric alcohol, the diol, and the triol are all hydrophilic. The compositions are presented herein below in table 1B. Physical parameters were evaluated after 24 hours of incubation at room temperature. Phase separation was assessed by visual inspection and grainy texture was assessed by skin feel evaluation, as described herein.

TABLE 1B

phase separation in formulations comprising paraffin wax, and a polar solvent (i.e., 2-propanol, propylene glycol or glycerin) without an oil

| Formulation | IDD287 | IDD286 | IDD288 | IDD224 |
|---|---|---|---|---|
| Ingredient | %w/w | %w/w | %w/w | %w/w |
| Paraffin wax 51-53 | 33.33 | 33.33 | 33.33 | 30.7 |
| 2-propanol | 66.67 | | | |
| Propylene glycol | - | 66.67 | - | - |
| Glycerin | - | - | 66.67 | 69.28 |
| Total | 100 | 100 | 100 | 100 |
| Ratio PW:polar solvent | 0.5:1 | 0.5:1 | 0.5:1 | 0.44:1 |
| Phase separation | YES | YES | YES | YES |
| Grainy | NO | NO | NO | NO |

*PW: Paraffin Wax.

Each formulation comprised of (i) 2- propanol (a monohydric alcohol), propylene glycol (a diol), or glycerin (a triol), respectively, as a polar phase at an amount of 66.67% or 69.28% w/w (i.e., the major phase), and (ii) a paraffin wax as a hydrophobic phase. As can be seen above in table 1B, none of the prepared formulations remained homogenous and phase separation was observed in each case. Additional formulations comprising a polar solvent and wax as tested in Table 1B, but at different amounts, were formulated. The amount of each ingredient was adjusted so that the wax was now the major component.

As shown in Table 1C, three formulations, comprising 2-propanol, propylene glycol, or glycerin at an amount of 33.33% w/w and paraffin wax 57-60 at an amount of 66.67% w/w, were evaluated. The compositions and observations are presented herein below in Table 1C.

Physical parameters were evaluated after 24 hours of incubation at room temperature. Phase separation was assessed by visual inspection and grainy texture was assessed by feel inspection.

TABLE 1C

formulations comprising paraffin wax, and a polar solvent (i.e., 2-propanol, propylene glycol or glycerin) without an oil

| Formulation | IDD283 | IDD284 | IDD285 |
|---|---|---|---|
| Ingredient | %w/w | %w/w | %w/w |
| Paraffin wax 57-60 | 66.67 | 66.67 | 66.67 |
| 2-propanol | - | 33.33 | - |
| Propylene glycol | 33.33 | - | - |
| Glycerin | - | - | 33.33 |
| Total | 100 | 100 | 100 |
| Ratio PW:polar solvent | 2:1 | 2:1 | 2:1 |
| Phase separation | YES | NO | NO |
| Grainy | NO | NO | NO |

*PW: Paraffin Wax.

As can be seen above in Table 1C, propylene glycol (a diol) combined with wax resulted in phase separation. However, surprisingly the formulations which comprised as a polar phase, 33.33%w/w, of 2-propanol, or glycerin, and mixed with melted paraffin wax as the hydrophobic phase (ratio between the wax to the polar solvent of 2:1) presented no phase separation after allowing the formulation to cool. This was unexpected given their corresponding hydrophilic and hydrophobic natures.

Without being bound by any theory or mechanism of action, it is thought that the molecular interaction of the wax molecules (e.g., providing a viscous strength to the hydrophobic phase) may prevent certain polar globules that became entrapped in wax from escaping the wax at room temperature when wax is the major or continuous phase. It is further suggested, without being bound by any theory or mechanism of action, that propylene glycol, a diol, is less compatible (or more incompatible) with paraffin wax hence the phase separation.

Thus, in one or more embodiments, provided herein is a monohydric alcohol or triol formulation, wherein the ratio between the wax and polar solvent is about or more than about 2:1.

### Example 2 - Formulations Comprising a Polar Solvent, Propylene Glycol, Hexylene Glycol, or 2-Propanol in Paraffin Wax and Mineral Oil

Formulations comprising a hydrophobic phase comprising paraffin wax (“PW”) 51-53 or 57-60 with mineral oil (“MO”) and a polar phase (propylene glycol or hexylene glycol or 2-propanol) were prepared without an emulsifier or other amphiphilic compound and without a fatty acid or fatty alcohol. Physical properties (phase separation and presence of grainy feeling) of the formulations were evaluated after 24 hours from preparation of the formulations. The tested formulations and results are shown in Tables 2A and 2B. In these formulations, the hydrophobic phase is the major phase.

TABLE 2A

Formulations comprising a glycol, paraffin wax 51-53 and mineral oil

| Formulation | IDD230^{#} | IDD232^{#} | IDD233^{#} |
|---|---|---|---|
| Ingredient | %w/w | %w/w | %w/w |
| Mineral oil (MO) | 31.96 | 38.56 | 38.56 |
| Paraffin wax 51-53 | 31.96 | 40.95 | 40.95 |
| Propylene glycol (PG) | 36.08 | 20.50 | - |
| Hexylene glycol (HG) | - | - | 20.50 |
| Total | 100 | 100 | 100 |
| Ratio PW:PG/HG | 0.88:1 | 2:1 | 2:1 |
| Ratio PW: MO | 1:1 | 1.06:1 | 1.06:1 |
| Ratio MO: PG/HG | 0.88:1 | 1.88:1 | 1.88:1 |
| Ratio PW+MO: PG/HG | 1.77:1 | 3.8:1 | 3.8:1 |
| Phase separation | YES | YES | YES |
| Grainy | YES | YES | YES |

*PW: Paraffin Wax; MO: Mineral Oil; PG: Propylene Glycol; HG: Hexylene Glycol

As can be seen above in table 2A, all these formulations were physically unstable presenting phase separation and having a grainy texture (the formulations were not smooth but rather grainy).

TABLE 2B

formulations comprising propylene glycol or 2-propanol with paraffin wax 57-60 and mineral oil

| Formulation | IDD281 | 7A | IDD282 |
|---|---|---|---|
| Ingredient | %w/w | %w/w | %w/w |
| Mineral oil (MO) heavy | 40.00 | 37.67 | 40.00 |
| Paraffin wax 57-60 | 40.00 | 40.00 | 40.00 |
| Propylene glycol (PG) | - | - | 20.00 |
| 2-propanol | 20.00 | 20.00 | - |
| Dox | - | 2.33 | - |
| Total | 100 | 100 | 100 |
| Ratio PW:polar solvent | 2:1 | 2:1 | 2:1 |
| Ratio PW: MO | 1:1 | 1.06:1 | 1:1 |
| Ratio MO:polar solvent | 2:1 | 1.88:1 | 2:1 |
| Ratio PW+MO: polar solvent | 4:1 | 3.88:1 | 4:1 |
| Phase separation | NO | NO | YES |
| Grainy | NO | NO | NO |

^{∗}PW: Paraffin Wax; MO: Mineral Oil; PG: Propylene Glycol; polar solvent: PG or 2-propanol.

As can be seen in Table 2B above, phase separation was likewise observed when propylene glycol is combined with a different higher melting point paraffin wax and mineral oil.

However, when 2-propanol was combined with the same hydrophobic ingredients surprisingly no phase separation was observed. Similarly, addition of doxycycline to a 2-propanol formulation resulted in a homogenous and smooth composition. Without being bound by any theory, the presence of mineral oil dilutes the wax to form a hydrophobic phase, can strengthen the hydrophobicity of the hydrophobic phase and thus the potential for the hydrophilic polar phase to separate may be greater. Surprisingly, despite the potential to separate in the absence of any emulsifier the propanol formulation did not present phase separation.

These results cannot per se be simply explained on the basis of the melting or boiling points of the polar solvents. Propanol has the lowest and glycerol the highest melting or boiling point with propylene glycol and hexylene glycol being in the middle. Nor can they be simply explained in terms of the number of OH groups again as propylene and hexylene glycol are in the middle.

### Example 3 - Formulations Comprising Glycerin and Propylene Glycol in Paraffin Wax 51-53 and Mineral Oil

Compositions comprising a polar phase that is a mixture of glycerin and propylene glycol, and a hydrophobic phase that is a mixture of paraffin wax 51-53 and mineral oil were prepared and their physical properties (i.e., phase separation and presence of grains) were evaluated on the day of formulation manufacturing.

TABLE 3

formulations comprising glycerin, propylene glycol, paraffin wax, and mineral oil

| Formulation | IDD228^{#} | IDD229^{#} |
|---|---|---|
| Ingredient | %w/w | %w/w |
| Mineral oil | 14.9 | 25.6 |
| Paraffin wax 51-53 | 34.18 | 34.16 |
| Propylene glycol (PG) | 21.36 | 21.35 |
| Glycerin | 29.5 | 18.86 |
| Total | 100 | 100 |
| Ratio PW:PG+Glycerin | 0.68:1 | 0.86:1 |
| Ratio PW:glycerin | 1.15:1 | 1.8:1 |
| Ratio PW:PG | 1.6:1 | 1.6:1 |
| Ratio PW: MO | 2.29:1 | 1.33:1 |
| Ratio MO: PG+Glycerin | 0.29:1 | 0.63:1 |
| Ratio MO:glycerin | 0.5:1 | 1.36:1 |
| Ratio PW+MO: PG+Glycerin | 0.96:1 | 1.48:1 |
| Phase separation | YES | YES |
| Grainy | YES | YES |

*PW: Paraffin Wax; MO: Mineral Oil; PG: Propylene Glycol

The above tested compositions exhibited a phase separation and grainy texture. Without being bound by any theory or mechanism of action, it is thought that the presence of glycerol with propylene glycol may not able to prevent the propylene glycol causing phase separation when the hydrophilic phase is the major phase. Additionally or alternatively, it is further suggested, without being bound by any theory or mechanism of action, that the phase separation may be encouraged due to the higher amount of the polar phase present, relative to the oil diluted wax hydrophobic phase, resulting in a reduced capacity of the oil diluted wax to entrap and stabilize the polar phase to achieve a homogenous composition.

### Example 4A - Formulations Comprising Glycerin, Mineral Oil and Paraffin Wax Having Various Melting Points

Formulations comprising glycerin, mineral oil, paraffin wax 42-44 possessing a melting point of 42° C. to 44° C., and paraffin wax 51-53 possessing a melting point of 51° C. to 53° C., and paraffin wax 57-60 possessing a melting point of 57° C. to 60° C. were prepared. The physical parameters of the formulations were evaluated thereafter preparation of the formulations on the day of formulation manufacturing.

TABLE 4A

Formulations comprising mineral oil, glycerin and various paraffin wax melting points

| Formulation | IDD261 | IDD235 | IDD231^{#} | IDD203 |
|---|---|---|---|---|
| Ingredient | %w/w | %w/w | %w/w | %w/w |
| Mineral oil | 37.67 | 40.00 | 31.95 | 37.67 |
| Paraffin wax 42-44 | 40.00 | - | - | - |
| Paraffin wax 51-53 | - | 40.00 | 31.95 | - |
| Paraffin wax 57-60 | - | - | - | 40.00 |
| Glycerin | 20.00 | 20.00 | 36.08 | 20.00 |
| Dox | 2.33 | - | - | 2.33 |
| Total | 100 | 100 | 100 | 100 |
| Ratio PW:glycerin | 2:1 | 2:1 | 0.88:1 | 2:1 |
| Ratio PW:MO | 1.06:1 | 1:1 | 1:1 | 1.06:1 |
| Ratio MO: glycerin | 1.88:1 | 2:1 | 0.88:1 | 1.88:1 |
| Ratio PW+MO:glycerin | 3.88:1 | 4:1 | 1.77:1 | 3.88:1 |
| Phase separation | NO | NO | YES | NO |
| Grainy | NO | NO | YES | NO |

*PW: paraffin wax; MO: Mineral oil

No phase separation was observed in formulations IDD261, IDD235 and IDD203 containing 20% glycerin and 40% of paraffin wax 42-44, paraffin wax 51-53 or paraffin wax 57-60, respectively. Phase separation was seen in formulation IDD231 containing 31.95% mineral oil, 31.95% paraffin wax and 36.08% paraffin wax.

Without being bound by any theory, it is hypothesized that the phase separation may be encouraged due to the higher amount of the polar phase present (i.e., glycerin) relative to the amount of the hydrophobic phase (oil diluted wax) or relative to merely the wax, resulting in a reduced capacity of the hydrophobic phase to entrap and stabilize the polar phase to achieve a homogenous composition.

It is further hypothesized, without being bound by any theory, that the weight ratio between the one or more ingredients in the formulations may affect the overall capacity of the formulations to maintain homogeneity. Formulations IDD235 and IDD231 having similar ingredients presented different physical properties, wherein no phase separation was observed for IDD235, versus a phase separation shown in formulation IDD231. The ratio between the wax to polar solvent, and mineral oil to polar solvent was two times higher in the IDD235 formulation than corresponding ratios in the IDD231 formulation. Consequently, the hydrophobic phase (i.e., wax plus oil) to hydrophilic phase ratio in the IDD235 formulation was also more than two times higher than the corresponding hydrophobic phase to hydrophilic phase ratio in the IDD231 formulation. Formulations IDD261, and IDD 203 presented ingredients' ratios similar to those of IDD235. Thus, in one or more embodiments, the ratio between the hydrophobic phase to hydrophilic phase in the herein disclosed compositions is more than about 1.77:1, or about or more than about 3.88: 1. In one or more embodiments, the ratio between the wax to polar solvent in the herein disclosed compositions is more than about 0.88:1, or about or more than about 2:1. In one or more embodiments, the ratio between the oil to polar solvent in the herein disclosed compositions is more than about 0.88:1, or about or more than about 1.88:1.

Without being bound by any theory, it is hypothesized that paraffin wax fractions each having various average melting points and which are solid at room temperature are suitable for use with glycerin. Thus, in one or more embodiments, the wax may have a melting point of about or more than 40° C. In one or more other embodiments, the wax may have a melting point of about or above 44° C.

### Example 4B - Formulations Comprising Glycerin, Mineral Oil and Various Waxes

Formulations comprising glycerin, mineral oil, and a wax comprising beeswax, hydrogenated castor oil or microcrystalline wax were prepared. The physical parameters of the formulations were evaluated 72 hours thereafter.

TABLE 4B

Formulations comprising mineral oil, glycerin and various waxes

| Formulation | 2A | 2B | 2D | IDD203 |
|---|---|---|---|---|
| Ingredient | %w/w | %w/w | %w/w | %w/w |
| Mineral oil | 37.67 | 37.67 | 37.67 | 37.67 |
| Beeswax | 40.00 | - | - | - |
| Hydrogenated castor oil | - | 40.00 | - | - |
| Emulsifying wax | - | - | - | - |
| Microcrystalline wax (76.7-82.2) | - | - | 40.00 | - |
| Paraffin wax 57-60 | - | - | - | 40.00 |
| Glycerin | 20.00 | 20.00 | 20.00 | 20.00 |
| Dox | 2.33 | 2.33 | 2.33 | 2.33 |
| Total | 100 | 100 | 100 | 100 |
| Ratio Wax:glycerin | 2:1 | 2:1 | 2:1 | 2:1 |
| Ratio wax:MO | 1.06:1 | 1.06:1 | 1.06:1 | 1.06:1 |
| Ratio MO: glycerin | 1.88:1 | 1.88:1 | 1.88:1 | 1.88:1 |
| Ratio wax+MO:glycerin | 3.88:1 | 3.88:1 | 3.88:1 | 3.88:1 |
| Phase separation | NO | YES | NO | NO |
| Grainy | NO | YES | NO | NO |

*MO: Mineral oil

No phase separation was observed in formulations 2A, 2D and IDD203, containing beeswax, microcrystalline wax and paraffin wax 57-60, respectively. To the contrary, formulation 2B containing hydrogenated castor oil resulted in a non-homogenous grainy composition. It may be that larger crystal structures seen with hydrogenated castor oil may contribute to a grainy feel.

Without being bound by any theory, it is hypothesized that the homogeneity of the compositions comprising microcrystalline wax and paraffin wax may be explained by the fact that these waxes are hydrocarbon-based waxes that are compatible with mineral oil, a hydrocarbon-based solvent. This compatible oil diluted-wax system seems to entrap or encapsulate or coat glycerin thereby avoiding phase separation. Hydrogenated castor oil, on the other hand, is a saturated vegetable oil wax that is less compatible with hydrocarbon-based oils such as mineral oil. The reduced compatibility may account for the phase separation observed in composition 2B. Thus, in one or more embodiments, the wax may be a hydrocarbon-based wax comprising a paraffin wax, or a microcrystalline wax. In one or more embodiments, the wax comprises a beeswax, which or can in one or more embodiments form smaller particles or crystals than hydrogenated castor oil. In one or more embodiments, the wax is substantially free, essentially free or free of hydrogenated castor oil.

### Example 4C - A Formulation Comprising Glycerin, Mineral Oil and a Combination of Two Waxes

A formulation comprising glycerin, mineral oil, and a combination of two waxes comprising beeswax and paraffin wax (57-60) was prepared. The physical parameters of the formulation were evaluated 72 hours thereafter.

TABLE 4C

Formulation comprising mineral oil, glycerin and a combination of two waxes

| Formulation | 3A |
|---|---|
| Ingredient | %w/w |
| Mineral oil | 37.67 |
| Beeswax | 20.00 |
| Paraffin wax 57-60 | 20.00 |
| Glycerin | 20.00 |
| Dox | 2.33 |
| Total | 100 |
| Ratio Wax:glycerin | 2:1 |
| Ratio wax:MO | 1.06:1 |
| Ratio MO: glycerin | 1.88:1 |
| Ratio wax+MO:glycerin | 3.88:1 |
| Phase separation | NO |
| Grainy | NO |

No phase separation was seen in formulation 3A comprising a combination of beeswax and Paraffin wax 57-60. The formulation obtained was homogenous and smooth (not grainy). Thus, in one or more embodiments, the composition comprises a combination of at least two waxes.

### Example 5A - Formulations Comprising Glycerin in Various Concentrations of Paraffin Wax and Mineral Oil Over Time.

Compositions with glycerin (20% w/w) in a mixture of paraffin wax 57-60 (1-70% w/w) and mineral oil (to 100%) were prepared. Formulations IDD203, IDD209-IDD212, IDD262, IDD263, IDD267 and IDD268 were incubated for 24 hours at room temperature (under dark conditions). Formulations 1A-1D were incubated for 72 hours at room temperature. The physical parameters of the formulations were evaluated thereafter.

TABLE 5A

formulations comprising glycerin and increasing mineral oil and decreasing paraffin wax concentrations

| Formulation | 1C | 1B | 1A | 1D | IDD203 | IDD209 | IDD210 | IDD211 |
|---|---|---|---|---|---|---|---|---|
| Ingredient | %w/w | %w/w | %w/w | %w/w | %w/w | %w/w | %w/w | %w/w |
| Mineral oil | 7.67 | 17.67 | 27.67 | 32.67 | 40.00 | 45.00 | 50.00 | 55.00 |
| Paraffin wax 57-60 | 70.00 | 60.00 | 50.00 | 45.00 | 40.00 | 35.00 | 30.00 | 25.00 |
| Glycerin | 20.00 | 20.00 | 20.00 | 20.00 | 20.00 | 20.00 | 20.00 | 20.00 |
| Dox | 2.33 | 2.33 | 2.33 | 2.33 | - | - | - | - |
| Total | 100.00 | 100.00 | 100.00 | 100.00 | 100.00 | 100.00 | 100.00 | 100.00 |
| Ratio PW:MO | 9.1:1 | 3.4:1 | 1.8:1 | 1.4:1 | 1:1 | 0.77:1 | 0.6:1 | 0.5:1 |
| Ratio PW: glycerin | 3.5:1 | 3:1 | 2.5:1 | 2.25:1 | 2:1 | 1.75:1 | 1.5:1 | 1.25:1 |
| Ratio MO:glycerin | 0.38:1 | 0.88:1 | 1.38:1 | 1.63:1 | 2:1 | 2.25:1 | 2.5:1 | 2.75:1 |
| Ratio MO+PW: Glycerin | 4:1 | 4:1 | 4:1 | 4:1 | 4:1 | 4:1 | 4:1 | 4:1 |
| Phase separation | YES | YES | NO | NO | NO | NO | NO | NO |
| Grain | YES | YES | YES | NO | NO | NO | NO | NO |

*PW: paraffin wax; MO: Mineral oil.

TABLE 5A Cont.

formulations comprising glycerin and increasing mineral oil and decreasing paraffin wax concentrations

| Formulation | IDD212 | IDD262 | IDD263 | IDD267 | IDD268 |
|---|---|---|---|---|---|
| Ingredient | %w/w | %w/w | %w/w | %w/w | %w/w |
| Mineral oil | 60.00 | 62.67 | 67.67 | 72.67 | 76.67 |
| Paraffin wax 57-60 | 20.00 | 15.00 | 10.00 | 5.00 | 1.00 |
| Glycerin | 20.00 | 20.00 | 20.00 | 20.00 | 20.00 |
| Dox | - | 2.33 | 2.33 | 2.33 | 2.33 |
| Total | 100.00 | 100.00 | 100.00 | 100.00 | 100.00 |
| Ratio PW:MO | 0.33:1 | 0.24:1 | 0.15:1 | 0.07:1 | 0.013:1 |
| Ratio PW: glycerin | 1:1 | 0.75:1 | 0.5:1 | 0.25:1 | 0.05:1 |
| Ratio MO:glycerin | 3:1 | 3.13:1 | 3.38:1 | 3.63:1 | 3.83:1 |
| Ratio MO+PW: Glycerin | 4:1 | 4:1 | 4:1 | 4:1 | 4:1¹⁰ |
| Phase separation | NO | NO | NO | NO | YES |
| Grain | NO | NO | NO | NO | NO |

Phase separation was not observed at 20% glycerin with mineral oil at concentrations of 27.67% and up to 72.67% and paraffin wax 57-60 at concentrations of 50% and decreased to 5%. Nevertheless, a composition comprising only 1% paraffin wax 57-60 and mineral oil 76.67% and compositions comprising high amounts of paraffin wax 57-60 (60% or 70%) and low amounts of mineral oil (17.67% or 7.67%, respectively) presented a phase separation. These findings suggest that the viscosity of the hydrophobic phase and therefore of the formulation can be modified whilst the polar phase remains constant by adding mineral oil and reducing wax (or reducing mineral oil and adding wax) until a certain concentration without significantly affecting the physical stability.

In addition, phase separation was not observed for formulation IDD203 after incubation for 6 months at room temperature.

Without being bound by any theory, it is hypothesized that glycerin is entrapped within the composition by the presence of oil diluted wax across a wide range of wax and oil variations. The results surprisingly show that even a low concentration of wax of 5% diluted in oil is sufficient to maintain the glycerin distributed or entrapped within the oil diluted wax hydrophobic phase of the composition. On the other hand, high wax concentrations accompanied with low amounts of oils may result in an excess of solid waxy material that cannot be sufficiently dissolved in the hydrophobic phase. Another way to look at this without being bound by any theory is that increasing low amounts of liquid oil can improve the fluidity and flexibility of the wax allowing the hydrophobic phase to better contain or entrap the polar phase. Such a system with low oil and high wax may be less flexible and fluid and less capable or incapable of effectively entrapping the polar phase, thereby resulting in a phase separation. On the other hand too much oil and insufficient wax may be too fluid and flexible and allow for phase separation.

In one or more embodiments the composition comprises a wax at a concentration of above 1% and below 60% by weight of the composition. In one or more embodiments, the composition comprises a wax in a concentration of about 5% to about 50% by weight of the composition. For e.g. about 5% to about 10% by weight, about 10% to about 15% by weight, about 15% to about 20% by weight, about 20% to about 25% by weight, about 25% to about 30% by weight, about 30% to about 35% by weight, about 35% to about 40% by weight, about 40% to about 45% by weight, about 45% to about 50% by weight, about 5% to about 15% by weight, about 15% to about 25% by weight, about 25% to about 35% by weight, about 35% to about 45% by weight, and about 40% to about 50% by weight of the composition. In one or more embodiments the composition comprises a wax in a concentration of about 5% by weight of the composition or about 10% by weight, or about 15% by weight, or about 20% by weight, or about 25% by weight, or about 30% by weight, or about 35% by weight, or about 40% by weight, or about 45% by weight, or about 50% by weight of the composition. In one or more embodiments, the composition comprises a hydrophobic solvent in a concentration of about 27% to about 73% by weight of the composition. For e.g. about 27% to about 35% by weight, about 35% to about 40% by weight, about 40% to about 45% by weight, about 45% to about 50% by weight, about 50% to about 55% by weight, about 55% to about 60% by weight, about 60% to about 65% by weight, about 65% to about 70% by weight, and about 70% to about 73% by weight of the composition. In one or more embodiments, the composition comprises a hydrophobic solvent in a concentration of about 27% by weight of the composition or about 30% by weight, or about 35% by weight, or about 40% by weight, or about 45% by weight, or about 50% by weight, or about 55% by weight, or about 60% by weight, or about 65% by weight, or about 70% by weight, or about 73% by weight of the composition.

### Example 5B - Formulations Comprising Doxycycline-Glycerin in Mineral Oil and Increasing Amounts of Microcrystalline Wax

Doxycycline formulations_containing an organic polar phase (e.g., glycerol) and an organic hydrophobic phase (e.g., Octyl dodecanol (OD) and Caprylic/capric Triglycerides (MCT) with decreasing amounts of mineral oil (MO) and increasing amounts of microcrystalline wax (MW) were prepared and evaluated after 72 hours at room temperature.

TABLE 5B

formulations comprising doxycycline-glycerin in decreasing amounts of mineral oil and increasing amounts of microcrystalline wax

| Formulation | 19A | 19D | 19B | IDD336 | 19C |
|---|---|---|---|---|---|
| Ingredient | %w/w | %w/w | %w/w | %w/w | %w/w |
| Mineral oil (MO) | 51.84 | 46.84 | 36.84 | 16.84 | 1.84 |
| Octyl dodecanol (OD) | 12.00 | 12.00 | 12.00 | 12.00 | 12.00 |
| Caprylic/capric Triglycerides (MCT) | 20.00 | 20.00 | 20.00 | 20.00 | 20.00 |
| Microcrystalline wax 76.7-82.2 (MW) | 5.00 | 10.00 | 20.00 | 40.00 | 55.00 |
| Glycerin | 10.00 | 10.00 | 10.00 | 10.00 | 10.00 |
| Doxycycline hyclate | 1.16 | 1.16 | 1.16 | 1.16 | 1.16 |
| Total | 100.00 | 100.00 | 100.00 | 100.00 | 100.00 |
| Ratio MW:Glycerin | 1:2 | 1:1 | 1:0.5 | 1:0.25 | 1:0.18 |
| Ratio MW: MO+OD+MCT | 1:16.8 | 1:7.9 | 1:3.44 | 1:1.22 | 1:0.6 |
| Glycerin : MO+OD+MCT | 1:8.38 | 1:7.9 | 1:6.9 | 1:4.9 | 1:3.4 |
| Ratio MW+ MO+OD+MCT: glycerin | 8.88:1 | 8.88:1 | 8.88:1 | 8.88:1 | 8.88:1 |
| Phase separation | YES (liquid) | NO | NO | NO | NO |
| Grainy | NO | YES | NO | NO | NO |

MO: Mineral oil; MW: Microcrystalline wax; OD:Octyl dodecanol; MCT: Caprylic/capric Triglycerides (MCT).

Formulation 19A with 5% microcrystalline wax and a wax:hydrophobic solvent ratio of 1:16.8 resulted in a liquid composition.

Formulations 19D, 19B, IDD336 and 19C with 10%, 20%, 40% and 55% microcrystalline wax and a wax:hydrophobic solvent ratio of 1:7.9, 1:3.4, 1:1.2 and 1:0.6, respectively resulted in homogeneous oil-gel compositions.

Without being bound by any theory, it is speculated that a low amount of wax is insufficient to entrap the formulation and form a gel composition. Thus, in one or more embodiments, the composition , comprises more than about 5% by weight of wax. For e.g. about 10% by weight of wax, about 15% by weight of wax, about 20% by weight of wax, about 25% by weight of wax, about 30% by weight of wax, about 35% by weight of wax, about 40% by weight of wax, about 45% by weight of wax, about 50% by weight of wax, or about 55% by weight of wax. In one or more embodiments, the composition comprises about 10% to about 55% wax by weight of the composition. For e.g. about 10% to about 20%, about 20% to about 30%, about 30% to about 40%, about 40% to about 50% or about 45% to about 55% of wax by weight of the composition. In one or more embodiments, the ratio between the wax and the hydrophobic solvent is about 1:3 to about 1:0.6. In one or more embodiments, the ratio between the wax and the hydrophobic solvent is about 1:3 or more than about 1:3. In one or more embodiments, the ratio between the wax and the hydrophobic solvent is about 1:8 to about 1:0.6. In one or more embodiments, the ratio between the wax and the hydrophobic solvent is about 1:8 or more than about 1:8.

### Example 5C - Formulations Comprising Doxycycline-Glycerin in Mineral Oil And Microcrystalline Wax With/Without Paraffin Wax

Doxycycline formulations_containing an organic polar phase (e.g., glycerol) and an organic hydrophobic solvent (e.g., Caprylic/capric Triglycerides (MCT), Mineral oil and Octyl dodecanol (OD) or Cyclomethicone) with microcrystalline wax (MW) alone or in combination with paraffin wax were prepared and evaluated after 72 hours at room temperature.

TABLE 5C

formulations comprising doxycycline-glycerin in mineral oil and microcrystalline wax with/without paraffin wax

| Formulation | IDD328 | IDD336 | IDD339 |
|---|---|---|---|
| Ingredient | %w/w | %w/w | %w/w |
| Mineral oil (MO) | 17.67 | 16.84 | 16.83 |
| Octyl dodecanol (OD) | - | 12.00 | 12.00 |
| Caprylic/capric Triglycerides (MCT) | 15.00 | 20.00 | 20.00 |
| Cyclomethicone (CYC) | 5.00 | - | - |
| Microcrystalline wax 76.7-82.2 (MW) | 40.00 | 40.00 | 20.00 |
| Paraffin wax (PW) 57-60 | - | - | 20.00 |
| Glycerin | 20.00 | 10.00 | 10.00 |
| Doxycycline hyclate | 2.33 | 1.16 | 1.17 |
| Total | 100.00 | 100.00 | 100.00 |
| Ratio MW/MW+PW: Glycerin | 1:0.5 | 1:0.25 | 1:0.25 |
| Ratio MW/MW+PW: HS | 1:0.94 | 1:1.22 | 1:1.22 |
| Glycerin: HS | 1:1.88 | 1:4.9 | 1:4.9 |
| Phase separation | NO | NO | NO |
| Grainy | NO | NO | NO |

MO: Mineral oil; MW:Microcrystalline wax; PW: Paraffin wax; OD:Octyl dodecanol; MCT: Caprylic/capric Triglycerides (MCT); CYC: Cyclomethicone; HS: Hydrophobic solvent (MO +/OD/MCT/CYC).

All formulations resulted in homogeneous smooth (not grainy) oil-gel compositions. 203 It was found that the mineral oil could be partly replaced by a combination of different hydrophobic solvents. For e.g._caprylic/capric triglycerides (MCT) with octyl dodecanol (OD) or cyclomethicone or caprylic/capric triglycerides (MCT) with cyclomethicone.

It was further found that the paraffin wax 57-60 could be fully or partly replaced by a different wax e.g. microcrystalline wax.

In addition, it was found that a certain amount of a polar solvent is required to solubilize the active agent with a sufficient excess to maintain solubility without re crystallization and precipitation. For e.g. 2.33% of doxycycline was dissolved in 20% glycerin (formulation IDD238) and 1.16% doxycycline was dissolved in 10%. Likewise 5% glycerin is capable of dissolving 0.58% doxycycline.

### Example 6 - Formulations Comprising Mineral Oil and Glycerin

Compositions with glycerin (35% w/w or 45% w/w) in a mixture of paraffin wax 57-60 (20 %w/w) and mineral oil (to 100%) were prepared. Physical parameters were evaluated after 24 hours of incubation at room temperature.

TABLE 6

formulations comprising glycerin, mineral oil and paraffin wax

| | | |
|---|---|---|
| Formulation Ingredient (%w/w) | IDD270 | IDD271 |
| Mineral oil | 45.00 | 35.00 |
| Paraffin wax 57-60 | 20.00 | 20.00 |
| Glycerin | 35.00 | 45.00 |
| Total | 100.00 | 100.00 |
| Ratio PW:MO | 0.44:1 | 0.57:1 |
| Ratio PW: glycerin | 0.57:1 | 0.44:1 |
| Ratio MO: glycerin | 1.28:1 | 0.77:1 |
| Ratio MO+PW:Glycerin | 1.85:1 | 1.22:1 |
| Phase separation | NO | Yes |
| Grain | NO | NO |

*PW: paraffin wax; MO: Mineral oil

As can be seen in table 6, a formulation containing 45% glycerin, 20% wax and 35% mineral oil presented a phase separation. In contrast, no phase separation was observed in a formulation that includes 35% glycerin in a 65% hydrophobic phase with 20% wax.

Without being bound by any theory, it may be that when the overall ratio between the hydrophobic phase to polar phase (glycerin) is about 1.2 or below in the absence of an emulsifier or other amphiphilic compound, entrapped glycerin can escape the hydrophobic phase. In other words ratios of wax to glycerin that are closer to 1:1 may be more effective in holding the composition together provided there is not an excess or an abundance of oil.

Thus, in one or more embodiments, the ratio of hydrophobic phase to polar phase is above about 1.2:1, or above about 1.85:1, or above about 1.6:1, or above about, 1.4:1. In one or more embodiments, the amount of glycerin is about or below about 35% and the amount of oil is also below 35%.

### Example 7 - Homogenous Dispersion of Glycerin in Formulations Comprising Mineral Oil And Paraffin Wax 57-60

The visual appearance of a formulation comprising glycerin (20 %w/w), mineral oil (67.67 %w/w), and Paraffin wax 57-60 (10 %w/w) was evaluated under microscopy (x100) on the day of formulation manufacturing. A dye (FD&C yellow 6 powder) which dissolves in glycerin, but not the hydrophobic phase, was introduced to the formulation. The results are summarized in Table 7 and FlG.1.

TABLE 7

a formulation comprising mineral oil, paraffin wax, glycerin and a dye selective for glycerin

| | |
|---|---|
| Formulation | IDD266^{#} |
| Ingredient | %w/w |
| Mineral oil | 69.28 |
| Paraffin wax 57-60 | 10.23 |
| Glycerin | 20.47 |
| FD&C yellow 6 powder | 0.05 |
| Total | 100 |
| Ratio PW:glycerin | 0.5:1 |
| Ratio PW:MO | 0.15:1 |
| Ratio MO:glycerin | 3.38:1 |
| Ratio PW+MO:glycerin | 3.88:1 |
| Phase separation | NO |
| Grainy | NO |

*PW: paraffin wax; MO: Mineral oil

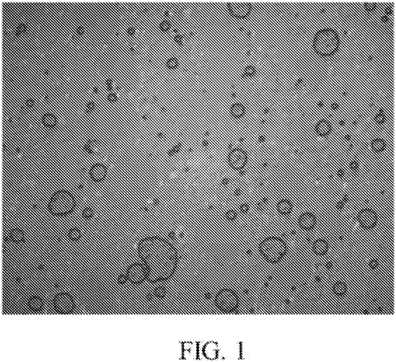

As can be seen in FIG. 1, the obtained formulation was homogenous, wherein the dyed glycerin was observed to be distributed evenly in the hydrocarbon oil diluted wax matrix (mineral oil and paraffin wax).

### Example 8A - Formulations Comprising Glycerin in Medium Chain Triglycerides (MCT) With/Without Mineral Oil and Paraffin Wax 57-60

Formulations comprising glycerin mixed with medium chain triglycerides (MCT) instead of, or in addition to mineral oil, were assessed on the day of formulation manufacturing. Formulations containing glycerin mixed in paraffin wax 57-60, MCT, and/or mineral oil were prepared according to Table 8.

TABLE 8A

formulations comprising MCT, glycerin, paraffin wax, with or without mineral oil

| Formulation | IDD241^{#} | IDD253^{#} | IDD254^{#} |
|---|---|---|---|
| Ingredient | %w/w | %w/w | %w/w |
| Mineral oil | - | 16.01 | 21.35 |
| Medium chain triglycerides (MCT) | 38.56 | 35.9 | 30.6 |
| Paraffin wax 57-60 | 40.9 | 26.68 | 26.68 |
| Glycerin | 20.47 | 21.35 | 21.35 |
| Total | 100 | 100 | 100 |
| Ratio PW:Glycerin | 2:1 | 1.24:1 | 1.24:1 |
| Ratio PW+MCT/MCT +MO:Glycerin | 3.88:1 | 3.68:1 | 3.68:1 |
| Phase separation | NO | NO | NO |
| Grainy | YES | NO | NO |

*PW: paraffin wax; MO: Mineral oil; MCT: Medium Chain triglycerides

For IDD253 and IDD254, it was found that replacement of hydrocarbons (mineral oil) by medium chain triglycerides (MCT) in part did not disturb the physical stability of the formulation. Phase separation was not observed, and the formulations obtained were homogenous and not grainy (smooth).

It was also observed that replacement of all the mineral oil by MCT oil (IDD241) did not result in phase separation. However, in contrast to partial replacement, full replacement led to a grainy formulation although the formulation was homogenous and did not show any phase separation.

Without being bound by any theory, MCT alone may be less compatible with the wax, resulting in a grainy formulation. The higher ratio of paraffin wax may also contribute.

### Example 8B - Formulations Comprising Different Types of Hydrophobic Solvents

Doxycycline formulations comprising paraffin wax 57-60 and glycerin mixed with soybean oil, isopropyl myristate, dimethicone or mineral oil, were prepared and assessed 72 hours following formulation manufacturing.

TABLE 8B

formulations comprising soybean oil, isopropyl myristate, dimethicone, cyclomethicone or mineral oil, as hydrophobic solvents

| Formulation | 4A | 4B | 4C | 16A | 16B | IDD203 |
|---|---|---|---|---|---|---|
| Ingredient | %w/w | %w/w | %w/w | %w/w | %w/w | %w/w |
| Soybean oil | 37.67 | - | - | - | - | - |
| Isopropyl myristate | - | 37.67 | - | 17.67 | - | - |
| Dimethicone | - | - | 37.67 | - | - | - |
| Cyclomethicone | - | - | - | - | 17.67 | - |
| Mineral oil (MO) | - | - | - | 20.00 | 20.00 | 37.67 |
| Paraffin wax (PW) 57-60 | 40.00 | 40.00 | 40.00 | 40.00 | 40.00 | 40.00 |
| Glycerin | 20.00 | 20.00 | 20.00 | 20.00 | 20.00 | 20.00 |
| Doxycycline hyclate | 2.33 | 2.33 | 2.33 | 2.33 | 2.33 | 2.33 |
| Total | 100.00 | 100.00 | 100.00 | 100.00 | 100.00 | 100.00 |
| Ratio PW:Glycerin | 2:1 | 2:1 | 2:1 | 2:1 | 2:1 | 2:1 |
| Ratio PW:HS | 1.06:1 | 1.06:1 | 1.06:1 | 1.06:1 | 1.06:1 | 1.06:1 |
| Ratio HS: Glycerin | 1.88:1 | 1.88:1 | 1.88:1 | 1.88:1 | 1.88:1 | 1.88:1 |
| Ratio HS+PW:glycerin | 3.88:1 | 3.88:1 | 3.88:1 | 3.88:1 | 3.88:1 | 3.88:1 |
| Phase separation | NO | YES | NO | NO | NO | NO |
| Grainy | YES | YES | YES | NO | YES | NO |

*PW: paraffin wax; HS: Hydrophobic solvent

It was found that replacement of hydrocarbons (mineral oil) by soybean oil (formulation 4A) or dimethicone did not disturb the physical stability of the formulation. Phase separation was not observed. However, the formulations obtained were grainy.

It was also observed that replacement of the mineral oil by isopropyl myristate (formulation 4B) resulted in phase separation and the formulation obtained was grainy. However, as can be seen in formulation 16A, in contrast to full replacement of mineral oil, partial replacement by isopropyl myristate and a lower amount led to a homogenous formulation that was not grainy.

Partial replacement of mineral oil with cyclomethicone (formulation 16B) led to a homogenous formulation that was grainy.

Without being bound by any theory, since soybean oil and isopropyl myristate are oils enriched with fatty acids they may be less compatible with paraffin wax, which is a hydrocarbon-based ingredient, resulting in a phase separation and/or grainy formulation. Cyclomethicone and dimethicone are silicone-based oils and thus may be less compatible with paraffin wax resulting in grainy formulations (4C and 16B). In formulations where mineral oil was partially replaced (formulations 16A and 16B), there was enough mineral oil in the formulation to dissolve the paraffin wax, which may account for the formulations homogeneity.

### Example 8C - Formulations Comprising Petrolatum With/Without Mineral Oil

Doxycycline formulations comprising glycerin mixed with petrolatum instead of or in addition to mineral oil, were assessed 72 hours following formulation manufacturing.

TABLE 8C

formulations comprising petrolatum with/without mineral oil

| Formulation | 5A | 5B |
|---|---|---|
| Ingredient | %w/w | %w/w |
| Mineral oil | - | 17.67 |
| Petrolatum | 37.67 | 20.00 |
| Paraffin wax 57-60 | 40.00 | 40.00 |
| Glycerin | 20.00 | 20.00 |
| Doxycycline hyclate | 2.33 | 2.33 |
| Total | 100.00 | 100.00 |
| Ratio PW:Glycerin | 2:1 | 2:1 |
| Ratio PW+(PET/PET +MO):Glycerin | 3.88:1 | 3.88:1 |
| Phase separation | NO | NO |
| Grainy | NO | NO |

*PW: paraffin wax; MO: Mineral oil; PET: petrolatum

It was found that partly or full replacement of mineral oil by petrolatum did not disturb the physical stability of the formulation. Phase separation was not observed, and the formulations obtained were homogenous and not grainy (smooth).

Without being bound by any theory, petrolatum is compatible with the wax (both being a hydrocarbon-based ingredient), resulting in a stable and smooth formulation.

### Example 9 - Formulations Comprising Propylene Glycol and Glycerin Mixed With Stearyl Alcohol (Fatty Alcohol) and Mineral Oil

The effects of fatty alcohol and mineral oil on formulations comprising propylene glycol and glycerin were examined. Lower concentrations (~5-12%) of mineral oil were added to assist in smoothing the texture of the formulations. Formulations containing about 20% w/w propylene glycol and glycerin (to 100%) dispersed in a mixture of stearyl alcohol (about 25% w/w) and mineral oil (between about 5 and about 11% w/w) were prepared and the physical stability of each formulation was assessed on the day of formulation manufacturing.

TABLE 9

formulations comprising propylene glycol, glycerin, stearyl alcohol, and lowconcentrations of mineral oil

| Formulation | IDD214^{#} | IDD215^{#} | IDD216^{#} |
|---|---|---|---|
| Ingredient | %w/w | %w/w | %w/w |
| Mineral oil | 5.33 | 8.54 | 11.74 |
| Stearyl alcohol (SA) | 26.68 | 26.68 | 26.68 |
| Glycerin | 46.62 | 43.4 | 40.2 |
| Propylene glycol (PG) | 21.35 | 21.35 | 21.35 |
| Total | 100 | 100 | 100 |
| Ratio SA: Glycerin | 0.57:1 | 0.6:1 | 0.66:1 |
| Ratio SA: PG | 1.25:1 | 1.25:1 | 1.25:1 |
| Ratio SA: PG +glycerin | 0.4:1 | 0.4:1 | 0.43:1 |
| Ratio SA: MO | 5:1 | 3.12:1 | 2.27:1 |
| Ratio SA+MO: PG+glycerin | 0.47:1 | 0.54:1 | 0.62:1 |
| Phase separation | NO | NO | NO |
| Grainy | YES | YES | YES |

*SA: stearyl alcohol; MO: Mineral Oil; PG: Propylene Glycol

Semi-solid formulations were formulated as described in Table 9. They were found to be homogenous, and no phase separation was observed. However, in all three compositions formulations were determined to be grainy and this was regardless of the increasing concentration of the mineral oil.

Without being bound by any theory, it is speculated that the grains in the above formulations may be primarily attributed to the presence of stearyl alcohol crystals or particles, and/or that too low concentrations of mineral oil were used.

It is further speculated, on the other hand without being bound by any theory, that the fatty alcohol (stearyl alcohol) may promote the homogeneity of formulations comprising glycols (propylene glycol).

### Example 10 - Formulations Comprising Propylene Glycol and Glycerin With Different Fatty Alcohols (Liquid e.g. Oleyl Alcohol/Oleic Acid and Solid e.g. Cetostearyl Alcohol/Stearyl Alcohol).

Formulations comprising propylene glycol and glycerin mixed with stearyl alcohol and oleic acid or oleyl alcohol were prepared. An additional formulation containing also cetostearyl alcohol was prepared and assessed on the day of formulation manufacturing.

TABLE 10

formulations comprising propylene glycol, glycerin, stearyl alcohol and oleyl alcohol or oleic acid

| Formulation | IDD218^{#} | IDD219^{#} | IDD220^{#} | IDD221^{#} | IDD222^{#} | IDD223^{#} |
|---|---|---|---|---|---|---|
| Ingredient | %w/w | %w/w | %w/w | %w/w | %w/w | %w/w |
| Propylene glycol (PG) | 21.35 | 21.35 | 21.35 | 21.35 | 21.5 | 26.28 |
| Glycerin | 29.5 | 29.5 | 40.2 | 29.5 | 29.35 | 20.4 |
| Stearyl alcohol | 24.55 | 29.89 | 27.75 | 34.16 | 33.78 | 30.7 |
| Oleic acid | 24.55 | - | - | - | - | 5.11 |
| Cetostearyl alcohol | - | - | - | - | - | 7.16 |
| Oleyl alcohol | - | 19.21 | 10.67 | 14.9 | 15.35 | 10.2 |
| Total | 100 | 100 | 100 | 100 | 100 | 100 |
| Ratio SA or SA+CSA: Glycerin | 0.83:1 | 1.01:1 | 0.69:1 | 1.15:1 | 1.15:1 | 1.5:1 1.85:1 |
| Ratio SA or SA+CSA: PG | 1.14:1 | 1.4:1 | 1.3:1 | 1.6:1 | 1.57:1 | 1.16:1 1.44:1 |
| Ratio SA or SA+CSA: Glycerin +PG | 0.48:1 | 0.58:1 | 0.45:1 | 0.67:1 | 0.66:1 | 0.65:1 0.8:1 |
| Ratio SA or SA+CSA:Oleic acid and/or Oleyl alcohol | 1:1 | 1.55:1 | 2.6:1 | 2.3:1 | 2.2:1 | 2:1 |
| Ratio SA+oleic acid and/or Oleyl alcohol: Glycerin +PG | 0.96:1 | 0.96:1 | 0.62:1 | 0.96:1 | 0.96:1 | 1.13:1 |
| Phase separation | NO | NO | NO | NO | NO | NO |
| Grainy | NO | NO | NO | NO | NO | NO |

*SA: stearyl alcohol; CSA: Cetostearyl alcohol; PG: Propylene Glycol

As shown in Table 10, the resulting semi-solid formulations were homogenous with no phase separation and were smooth (not grainy). The presence of significant amounts of liquid fatty alcohol or liquid fatty acid surprisingly eliminated the problems of using solid fatty alcohol alone.

Without being bound by any theory, liquid fatty acids and alcohols like oleic acid and oleyl alcohol may dissolve particles of solid fatty alcohols like stearyl alcohol and cetostearyl alcohol, thereby allowing stable, non-grainy compositions to be formulated that can also include diols.

### Example 11 - Formulations Comprising Hexylene Glycol and Glycerin in Stearyl Alcohol And Oleyl Alcohol

Formulations comprising hexylene glycol and glycerin in a mixture of stearyl alcohol and oleyl alcohol were prepared and physical parameters were evaluated on the day of formulation manufacturing.

TABLE 11

formulations comprising hexylene glycol, glycerin, stearyl alcohol and oleyl alcohol

| Formulation | IDD226^{#} | IDD227^{#} |
|---|---|---|
| Ingredient | %w/w | %w/w |
| Hexylene glycol (HG) | 21.5 | 20.47 |
| Glycerin | 29.35 | 28.33 |
| Stearyl alcohol | 32.76 | 35.83 |
| Oleyl alcohol | 16.38 | 15.35 |
| Total | 100 | 100 |
| Ratio SA: Oleyl alcohol | 2:1 | 2.33:1 |
| Ratio SA:Glycerin | 1.11:1 | 1.26:1 |
| Ratio SA:Glcerin+ HG | 0.64:1 | 0.73:1 |
| Ratio SA: HG | 1.52:1 | 1.75:1 |
| Ratio SA+ Oleyl alcohol: HG+glycerin | 0.96:1 | 1.04:1 |
| Phase separation | NO | NO |
| Grainy | NO | YES |

*SA: stearyl alcohol; HG: Hexylene Glycol

As shown in Table 11, semi-solid formulations obtained from the above compositions presented no phase separation. However, IDD226 was found to be smooth and not grainy, while IDD227 exhibited a grainy texture. The former had a lower level of stearyl alcohol and a higher level of liquid fatty alcohol. Without being bound by any theory, the grainy texture may be due to higher concentration of stearyl alcohol relative to oleyl alcohol and a ratio of over 2:1 may lead to a grainy formulation.

In one or more embodiments the ratio of solid wax to liquid wax is about 2:1 or less than 2:1.

### Example 12- Doxycycline Solubility in Various Solvents

The solubility of doxycycline was evaluated in the solvents listed in Table 12.

### Method

As shown in Table 12, an excess amount of doxycycline (as hyclate) was added to each of the solvents tested and stirred at room temperature for 72 hours. Each composition was subjected to centrifugation and the supernatant with dissolved active agent analyzed. The weight /weight percentage of active agent found dissolved in each solvent is shown below:

TABLE 12

doxycycline solubility in various solvents

| Solvent | Doxycycline content | |
|---|---|---|
| | % w/w Dox base | % w/w Dox hyclate |
| Dimethyl isosorbide | 3.57 | 4.12 |
| Ethanol | 2.73 | 3.15 |
| Glycerin | 15.84 | 18.28 |
| Hexylene glycol | 4.74 | 5.47 |
| Isopropanol | 1.44 | 1.67 |
| Isopropyl myristate | Not detected | Not detected |
| Oleic acid | Not detected | Not detected |
| Oleyl alcohol | 0.26 | 0.29 |
| Propylene carbonate | 0.68 | 0.78 |
| Propylene glycol | 29.60 | 34.17 |
| Transcutol | 14.12 | 16.30 |
| 1,3 propane-di-ol | 18.51 | 21.56 |
| Dimethyl sulfoxide | 21.75 | 25.34 |

As can be seen in Table 12 above, propylene glycol, dimethyl sulfoxide, 1,3 propane-di-ol, glycerin, and then transcutol provided the highest solubility at room temperature, in that order. There appears no simple connection between solubility and the number of OH (hydroxyl) groups in the molecule since hexylene glycol (a diol) showed much lower solubility for doxycycline.

### Example 13 -Doxycycline Stability in Various Solvents

The stability of doxycycline in various solvents was assessed. The saturated doxycycline solutions formulated in Example 12 were diluted 1:1 with corresponding solvents. An accelerated stability test was conducted. Briefly, each diluted solution was incubated at 70° C. for 14 hours and then subjected to analytical analysis using UPLC.

TABLE 13

doxycycline stability at various solvents as tested following storage at 70° C. for 14 hours

| Solvent | Approximate doxycycline concentration after dilution, %w/w | Doxycycline content, % | | |
|---|---|---|---|---|
| | | Total % of impurities after temperature challenge | 4-Epi | 6-Epi |
| Dimethyl isosorbide | 1.80 | 23.27 | 8.43 | 1.85 |
| Ethanol | 1.50 | 0.13 | 2.03 | 0.69 |
| Glycerin | 8.00 | ND | 2.04 | 0.54 |
| Hexylene glycol | 2.50 | 4.11 | 6.40 | 2.36 |
| Isopropanol | 0.70 | 0.10 | 1.62 | 0.70 |
| Oleyl alcohol | 0.15 | 1.29 | 4.89 | 0.56 |
| Propylene carbonate | 0.35 | 0.62 | 9.31 | 0.95 |
| Propylene glycol | 15.00 | ND | 2.04 | 0.54 |
| Transcutol | 7.00 | 0.11 | 4.21 | 0.58 |
| 1, 3 propane-di-ol | 5.89 | 0.19 | 0.14 | 0.04 |
| Dimethyl sulfoxide | 9.99 | 1.42 | 1.35 | 0.06 |

*Values normalized according to doxycycline initial content %.

As can be seen in Table 13 above, as reflected by appearance of 4epi in an accelerated test, doxycycline appears to be stable in 1,3 propane-di-ol, dimethyl sulfoxide, isopropanol, ethanol, propylene glycol, and then glycerol.

When taken together, the solubility results summarized in Example 12 and stability results presented herein (Example 13) suggests that it may be beneficial to use 1,3 propane-di-ol, dimethyl sulfoxide, glycerol and/or propylene glycol as suitable solvents for topical doxycycline compositions.

### Example 14- Doxycycline Stability in a Glycerin Solution

The stability of 2.33 w/w% doxycycline hyclate (equivalent to actual 2 w/w% doxycycline) dissolved in glycerin (to 100%) was tested following incubation for 6 months at 5° C., 25° C., or 40° C. (under dark conditions). A formulation shown in Table 14A was used for the study.

TABLE 14A

a glycerin composition of solubilized doxycycline (i.e., composition 1)

| | |
|---|---|
| Ingredient | %w/w |
| Glycerin | 97.67 |
| Doxycycline hyclate | 2.33 |
| Total | 100.00 |
| Color score at 5° C. | 0 |
| Color score at 25° C. | 1 |
| Color score at 40° C. | 2 |
| Phase separation | NO |
| Grainy | NO |

Color and grainy texture were also assessed at each test condition. The obtained formulation had a form of solution, wherein the doxycycline was dissolved in glycerin and no grains were detected under microscopy (results not shown). The visual appearance of the formulation after three months incubation at 5° C., 25° C., or 40° C. was also assessed. It appears at 5° C., there was a slight or no change in color, whereas at 25° C., color changed from a color score of 0 to 1 and at 40° C., the color of the formulation was changed from 0 to 2. Doxycycline (DOX assay) and degradant (4-epi) contents (w/w%) were evaluated after incubation at 40° C. by UPLC, the results of which are summarized in Table 14B and 14C.

TABLE 14B

doxycycline content (DOX assay, w/w%) in composition 1

| | | | | | | | |
|---|---|---|---|---|---|---|---|
| Time (months) | 0 | 0.5 | 1 | 2 | 3 | 5 | 6 |
| DOX assay (w/w%), 5° C. | 97.10 | NA | NA | NA | 97.10 | NA | 98.75 |
| DOX assay (w/w%), 25° C. | 97.10 | NA | 99.5 | NA | 97.30 | NA | 97.40 |
| DOX assay (w/w%), 40° C. | 97.10 | 99.01 | 98.75 | 96.70 | 94.39 | 94.57 | 92.24 |

NA= Not available

TABLE 14C

4-epidoxycycline content (w/w %) in composition 1

| | | | | | | | |
|---|---|---|---|---|---|---|---|
| Time (months) | 0 | 0.5 | 1 | 2 | 3 | 5 | 6 |
| 4-Epi (w/w%), 5° C. | <0.10 | NA | NA | NA | <0.10 | NA | <0.10 |
| 4-Epi (w/w%), 25° C. | <0.10 | NA | 0.13 | NA | 0.30 | NA | 0.55 |
| 4-Epi (w/w%), 40° C. | <0.10 | 0.37 | 0.73 | 1.45 | 2.45 | 3.52 | 4.35 |

NA= Not available

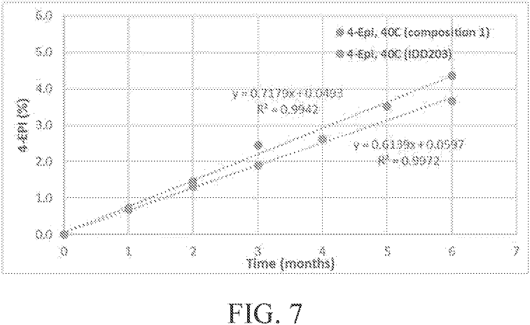

As can be seen in a dot plot in FIG. 7, doxycycline breakdown rate was constant presenting an average value of 0.72% 4-epi product formation per month at 40° C., and about 0.09% 4-epi product formation per month at 25° C.

### Example 15A - Formulations Comprising Doxycycline-Glycerin in Various Concentrations of Fatty Alcohol and Mineral Oil

Compositions comprising doxycycline dissolved in glycerin (20% w/w) and mixed within stearyl alcohol (10, 30, 35, 40 or 60 w/w%) and mineral oil (to 100%) were prepared. Samples IDD196, IDD 200 and IDD201 were incubated for 3 or 4 months at 5° C., 25° C., or 40° C. (under dark conditions). Following incubation, color was assessed and doxycycline content (DOX assay %w/w) and degradant content (4-epi, %w/w) were evaluated by UPLC. Samples 9A and 9B were incubated for 72 hours at room temperature and their physical properties were evaluated thereafter.

TABLE 15Ai

solubilized doxycycline composition with increasing stearyl alcohol and decreasing mineral oil concentrations

| Formulation | 9A | 9D | IDD196 | IDD200 | IDD201 | 9C | 9B |
|---|---|---|---|---|---|---|---|
| Ingredient | %w/w | %w/w | %w/w | %w/w | %w/w | %w/w | %w/w |
| Mineral oil | 67.67 | 56.67 | 47.67 | 42.67 | 37.67 | 27.67 | 17.67 |
| Stearyl alcohol (SA) | 10.00 | 20.00 | 30.00 | 35.00 | 40.00 | 50.00 | 60.00 |
| Glycerin | 20.00 | 20.00 | 20.00 | 20.00 | 20.00 | 20.00 | 20.00 |
| Doxycycline hyclate | 2.33 | 2.33 | 2.33 | 2.33 | 2.33 | 2.33 | 2.33 |
| Total | 100.00 | 100.00 | 100.00 | 100.00 | 100.00 | 100.00 | 100.00 |
| Ratio SA: glycerin | 0.5:1 | 1:1 | 1.5:1 | 1.75:1 | 2:1 | 2.5:1 | 3:1 |
| Ratio SA:MO | 0.15:1 | 0.35:1 | 0.62:1 | 0.8:1 | 1.06:1 | 1.8:1 | 3.4:1 |
| Color score at 5° C. | - | - | 0 | ND | 0 | - | - |
| Color score at 25° C. | - | - | 1 | ND | 1 | - | - |
| Color score at 40° C. | - | - | 2 (following 4 months) | ND | 2 (following 3 months) | - | - |
| Phase separation | NO (Liquid) | NO | NO | NO | NO | YES | YES |
| Grainy | NO | NO | NO | NO | NO | YES | NO |

*ND - not determined; MO: Mineral oil; SA: Stearyl alcohol

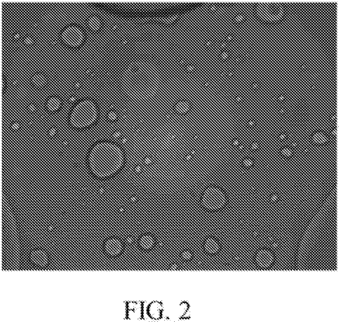

As shown in Table 15Ai, no phase separation was observed in formulations 9D, IDD196, IDD 200 and IDD201. These formulations were also found not to be grainy. FIG. 2 is a photographic image, showing a microscope evaluation of IDD201 formulation incubated for 3 months at 5° C. (light microscope x200). As shown in FIG. 2, doxycycline remained solubilized in the composition after the incubation. While no crystals of the doxycycline were observed under microscopy at any of the temperatures tested, color scores of IDD201 formulations were 0, 1, and 2, respectively, following 3 months of incubation at 5° C., 25° C., or 40° C.

As to formulation IDD196, color scores following 4 months of incubation at 5° C., 25° C., or 40° C. were 0, 1 and 2, respectively.

Formulation 9A comprising low amount of stearyl alcohol (10%) and high amount of mineral oil (67.67%) having a fatty alcohol: hydrophobic solvent ratio of 0.15:1, resulted in a liquid composition.

Formulations 9B and 9C comprising high amount of stearyl alcohol (60% and 50%, respectively) and low amount of mineral oil (17.67% and 27.67%, respectively), having a fatty alcohol: hydrophobic solvent ratio of 3.4:1 and 1.8:1, respectively, resulted in a phase separation.

Without being bound by any theory, it is speculated a wax (in this case a solid fatty alcohol): hydrophobic solvent ratio of 0.15:1 is not sufficient to solidify the composition and to form a gel like two-phase composition. Moreover, it is speculated that a wax: hydrophobic solvent ratios of 3.4:1 and 1.8:1 may be too high and that the phase separation of the 9B and 9C formulations may be primarily attributed to the presence of high amount of stearyl alcohol solids, and/or too low concentrations of mineral oil. Thus, in one embodiment the ratio of the wax: hydrophobic solvent is between about 0.35:1 to about 1.06:1 or between about 0.6:1 and about 1:1. In one or more embodiments the amount of fatty alcohol is between about 20% to about 40%.

TABLE 15Aii

doxycycline content (DOX assay, w/w%) in formulation IDD201

| Temp. condition | Zero time | 2 weeks | 1 month | 2 months | 3 months |
|---|---|---|---|---|---|
| 5° C. | 98.0 | - | 98.6 | 97.2 | 97.6 |
| 25° C. | 98.0 | - | 99.1 | 97.3 | 97.0 |
| 40° C. | 98.0 | 98.4 | 96.9 | 95.0 | 94.6 |

TABLE 15Aiii

4-epidoxycycline content (w/w %) in formulation IDD201

| Temp. condition | Zero time | 2 weeks | 1 month | 2 months | 3 months |
|---|---|---|---|---|---|
| 5° C. | 0.00 | - | 0.00 | 0.00 | 0.10 |
| 25° C. | 0.00 | - | 0.23 | 0.32 | 0.47 |
| 40° C. | 0.00 | 0.52 | 1.17 | 2.26 | 3.23 |
| Rate of 4-epi breakdown at 25° C. | 0.00 | | 0.23 per month | 0.16 per month | 0.156 per month |
| Rate of 4-epi breakdown at 40° C. | 0.00 | 0.26 per week | 1.17 per month | 1.13 per month | 1.07 per month |

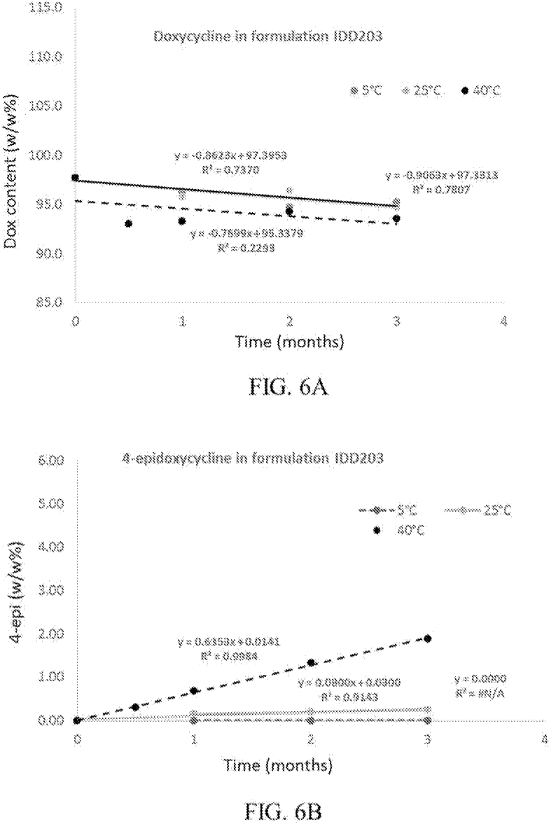

As can be seen in Tables 15Aii and 15Aiii and in the accompanying graphs at FIGS. 6A- 6B, IDD201 showed a linear doxycycline breakdown over time as assessed by the 4-epi levels.

### Example 15B Alternative Solid Fatty Alcohols

Compositions comprising doxycycline dissolved in glycerin (20% w/w) and mixed within solid fatty alcohols (Behenyl alcohol, Myristyl alcohol or stearyl alcohol) and mineral oil (to 100%) were prepared. Samples were incubated for 72 hours at room temperature and their physical properties were evaluated thereafter.

TABLE 15B

solubilized doxycycline composition with alternative solid fatty alcohols

| Formulation | 14A | 14B | IDD201 |
|---|---|---|---|
| Ingredient | %w/w | %w/w | %w/w |
| Mineral oil | 37.67 | 37.67 | 37.67 |
| Behenyl alcohol (SFA) (c22) | 40.00 | - | - |
| Myristyl alcohol (SFA) (c14) | - | 40.00 | - |
| Stearyl alcohol (SFA) (c18) | - | - | 40.00 |
| Glycerin | 20.00 | 20.00 | 20.00 |
| Doxycycline hyclate | 2.33 | 2.33 | 2.33 |
| Total | 100.00 | 100.00 | 100.00 |
| Ratio SFA: glycerin | 2:1 | 2:1 | 2:1 |
| Ratio SFA:MO | 1.06:1 | 1.06:1 | 1.06:1 |
| Phase separation | YES | NO | NO |
| Grainy | NO | NO | NO |

MO: Mineral oil; SFA: Solid fatty alcohol

As shown in Table 15B, phase separation was observed in formulation 14A containing behenyl alcohol. However, formulations 14B and IDD201 containing the same amount of an alternative solid fatty alcohol (Myristyl alcohol and Stearyl alcohol, respectively), were homogenous. These formulations were also found not to be grainy.

Without being bound by any theory, it is speculated that fatty alcohols with longer carbon chain (e.g. above c20) may not be suitable and contribute to phase separation. This may be due to their long structure or higher melting point. Thus, in one or more embodiments, the fatty alcohol comprises a fatty alcohol having between 14 and 20 carbon atoms in its backbone. In one or more embodiments, the fatty alcohol comprises a fatty alcohol having between 14 and 18 carbon atoms in its backbone. In one or more embodiments, the fatty alcohol comprises a combination of fatty alcohols comprising a fatty alcohol having between 14 and 20 carbon atoms in its backbone and a fatty alcohol having at least 20 carbon atoms in its backbone.

### Example 15C - Alternative Liquid Fatty Alcohols

Compositions comprising doxycycline dissolved in glycerin (20% w/w) and mixed within liquid fatty alcohols (Octyl dodecanol or Oleyl alcohol) and paraffin wax 57-60 (40%) were prepared. Samples were incubated for 72 hours at room temperature and their physical properties were evaluated thereafter.

TABLE 15C

solubilized doxycycline composition with alternative liquid fatty alcohols

| Formulation | 15B | 15C |
|---|---|---|
| Ingredient | %w/w | %w/w |
| Octyl dodecanol (LFA) | 37.67 | - |
| Oleyl alcohol (LFA) | - | 37.67 |
| Paraffin wax 57-60 | 40.00 | 40.00 |
| Glycerin | 20.00 | 20.00 |
| Doxycycline hyclate | 2.33 | 2.33 |
| Total | 100.00 | 100.00 |
| Ratio PW:Glycerin | 2:1 | 2:1 |
| Ratio PW: LFA | 1.06:1 | 1.06:1 |
| Ratio PW + LFA: glycerin | 3.88:1 | 3.88:1 |
| Phase separation | NO | NO |
| Grainy | YES | YES |

MO: Mineral oil; PW: Paraffin wax 57-60; LFA: Liquid fatty alcohol

As shown in Table 15C, no phase separation was observed in formulations 15B and 15C containing the liquid fatty alcohols Octyl dodecanol and Oleyl alcohol, respectively. However, these formulations were found to be grainy.

Without being bound by any theory, it is speculated that the liquid fatty alcohols may be less compatible with the paraffin wax as compared to mineral oil. Octyl dodecanol is a long chain fatty alcohol with an hydroxyl group in the middle of the carbon chain. Oleyl alcohol is a long chain fatty alcohol with a double bond in the middle of the carbon chain. These components may cause some structural disruption allowing grainy texture to result.

### Example 15D - Formulation Comprising Doxycycline-Glycerin in Paraffin Wax and Mineral Oil with a Gelling Agent

A doxycycline composition as shown in Table 15D was prepared. The composition comprises an organic polar phase (e.g., glycerol) and an organic hydrophobic phase (e.g., hydrocarbon ingredients such as a mineral oil (MO) and a paraffin wax (PW)) and a gelling agent (e.g. hydroxypropyl cellulose).

TABLE 15D

formulation comprising doxycycline-glycerin in paraffin wax and mineral oil with hydroxypropyl cellulose

| Formulation | 6A |
|---|---|
| Ingredient | %w/w |
| Mineral oil (MO) | 37.17 |
| Paraffin wax (PW) 57-60 | 40.00 |
| Glycerin | 20.00 |
| Hydroxypropyl cellulose (Klucel EF) | 0.50 |
| Doxycycline hyclate | 2.33 |
| Total | 100.00 |
| Ratio PW:Glycerin | 2:1 |
| Ratio PW: MO | 1.08:1 |
| Glycerin: MO | 1:1.86 |
| Phase separation | NO |
| Grainy | NO |

*PW: paraffin wax; MO: Mineral oil

As shown in Table 15D, formulation 6A containing a polymeric agent was homogenous and smooth. It showed no phase separation and no grains were detected.

### Example 16A - Formulations Comprising Doxycycline-Glycerin in Paraffin Wax and Mineral Oil with Increasing Glycerin Concentrations

As shown in Table 16A, compositions comprising doxycycline dissolved in various amounts of glycerin at (15%, 25%, 30%, 35%, or 45% w/w) and mixed with a mixture of paraffin wax 57-60 (40%w/w) and mineral oil (to 100%) were prepared and tested for phase separation and grains. Samples were then incubated for 2 months at 40° C. (under dark conditions). After incubation, color was assessed.

TABLE 16A

solubilized doxycycline composition with increasing glycerin and decreasing mineral oil concentrations

| | | | | | | |
|---|---|---|---|---|---|---|
| Formulation Ingredient (%w/w) | IDD204 | IDD203 | IDD205 | IDD269 | IDD206 | IDD207 |
| Mineral oil | 42.67 | 40.00 | 32.67 | 27.67 | 22.67 | 12.67 |
| Paraffin wax 57-60 | 40.00 | 40.00 | 40.00 | 40.00 | 40.00 | 40.00 |
| Glycerin | 15.00 | 20.00 | 25.00 | 30.00 | 35.00 | 45.00 |
| Doxycycline hyclate | 2.33 | ------ | 2.33 | 2.33 | 2.33 | 2.33 |
| Total | 100.00 | 100.00 | 100.00 | 100.00 | 100.00 | 100.00 |
| Ratio MO: glycerin | 2.84:1 | 2:1 | 1.3:1 | 0.9:1 | 0.65:1 | 0.28:1 |
| Ratio PW: glycerin | 2.66:1 | 2:1 | 1.6:1 | 1.33:1 | 1.14:1 | 0.88:1 |
| Ratio PW+MO:glycerin | 5.5:1 | 4:1 | 2.9:1 | 2.25:1 | 1.8:1 | 1.17:1 |
| Ratio PW:MO | 0.93:1 | 1:1 | 1.22:1 | 1.44:1 | 1.76:1 | 3.15:1 |
| Color score following 2 months at 40° C. | 0 | 0 | 0 | ND | 0 | 0 |
| Phase separation (RT) | NO | NO | NO | YES | YES | YES |
| Grainy | NO | NO | NO | YES | NO | NO |

*PW: paraffin wax; MO: Mineral oil; ND - not determined

### Example 16B - Formulation Comprising Low Amount Doxycycline-Glycerin

A composition comprising a low amount of doxycycline, a low amount of an organic polar phase (e.g., glycerol) and an organic hydrophobic phase (e.g., hydrocarbon ingredients such as a mineral oil (MO) and a paraffin wax (PW)) was prepared and evaluated after 72 hours at room temperature.

TABLE 16B

exemplary low-amount solubilized doxycycline composition

| Formulation | 8A | IDD204 | IDD203 | IDD270 |
|---|---|---|---|---|
| Ingredient | %w/w | %w/w | %w/w | %w/w |
| Mineral oil (MO) | 54.42 | 42.67 | 37.67 | 45.00 |
| Paraffin wax (PW) 57-60 | 40.00 | 40.00 | 40.00 | 20.00 |
| Glycerin | 5.00 | 15.00 | 20.00 | 35.00 |
| Doxycycline hyclate | 0.58 | 2.33 | 2.33 | - |
| Total | 100.00 | 100.00 | 100.00 | 100.00 |
| Ratio PW:Glycerin | 8:1 | 2.66:1 | 2:1 | 0.57:1 |
| Ratio PW: MO | 0.74:1 | 0.94:1 | 1.06:1 | 0.44:1 |
| MO:glycerin | 10.88:1 | 2.84:1 | 1.88:1 | 1.28:1 |
| Ratio PW+MO:glycerin | 18.88:1 | 5.5:1 | 3.88:1 | 1.85:1 |
| Dox: Glycerin | 1:8.6 | 1:6.4 | 1:8.6 | - |
| Phase separation | NO | NO | NO | NO |
| Grainy | NO | NO | NO | NO |

*PW: paraffin wax; MO:Mineral oil

It was found that a low amount of doxycycline was dissolved in a low amount of glycerin (8A formulation). No grains were detected under microscopy and no phase separation was observed. The ratio between doxycycline and glycerin in this composition was 1:9. Compositions IDD204 and IDD203 had higher amount of doxycycline and higher amounts of glycerin. The ratio between doxycycline and glycerin in these compositions was 1:6 and 1:9, respectively.

Without being bound by any theory, it is speculated that a certain amount of a polar solvent may be required to solubilize the active agent without re crystallization and precipitation. Thus, in one or more embodiments, the ratio between the active agent and the polar solvent is between about 1:6 to about 1:9.

It was further found that it is possible to arrive at compositions that show no phase separation and are not grainy over a wide range of glycerin amounts (formulations 8A, IDD204, IDD203, and IDD270 having 5%, 15%, 20% and 35% glycerin, respectively). Thus, in one or more embodiments the composition comprises a polar solvent in a concentration of about 5% to about 35% by weight of the composition. For e.g. about 5% to about 10% by weight, about 10% to about 15% by weight, about 15% to about 20% by weight, about 20% to about 25% by weight, about 25% to about 30% by weight or about 30% to about 35% by weight of the composition. It was also shown that in order to keep the formulation homogenous, when polar solvent levels are increased the ratio between the wax and hydrophobic solvent should be kept in a way that would enable the hydrophobic solvent to solubilize the wax, thereby creating a system that entraps or encompasses the glycerin, prevents phase separation and maintains composition homogeneity. For e.g. formulation IDD206 comprising higher glycerin levels (35%, see Table 16Ai) and a wax: hydrophobic solvent ratio of 1.76:1 resulted in phase separation. Without being bound by any theory, it is possible that there was not enough hydrophobic solvent to solubilize the wax and to create the appropriate system to entrap a high amount of polar solvent. To the contrary, formulation IDD270, containing the same amount of polar solvent but a ratio of wax:hydrophobic solvent of 0.44:1, formed a homogenous composition that was smooth (not grainy). Thus, in one or more embodiments the ratio between the wax and the hydrophobic solvent in the composition is less than about 1.8:1. For e.g. less than about 1.44:1, or is about 1.22:1 or less than about 1.22:1.

### Example 17A - Formulations Comprising Doxycycline-Glycerin in Mineral Oil and Paraffin Wax

A doxycycline carrier composition as shown in Table 17A was prepared. The composition comprises an organic polar phase (e.g., glycerol) and an organic hydrophobic phase (e.g., hydrocarbon ingredients such as a mineral oil (MO) and a paraffin wax (PW)). An exemplary composition entitled IDD203 is presented herein below in Table 17A.

TABLE 17A

exemplary solubilized doxycycline composition

| Formulation | IDD203 |
|---|---|
| Ingredient | %w/w |
| Mineral oil (MO) | 37.67 |
| Paraffin wax (PW) 57-60 | 40.00 |
| Glycerin | 20.00 |
| Doxycycline hyclate | 2.33 |
| Total | 100.00 |
| Ratio PW:Glycerin | 2:1 |
| Ratio PW: MO | 1.06:1 |
| Ratio MO:Glycerin | 1.88:1 |
| Ratio MO+PW:glycerin | 3.88:1 |
| Color score | 0 |
| Phase separation | NO |
| Grainy | NO |

*PW: paraffin wax; MO: Mineral oil

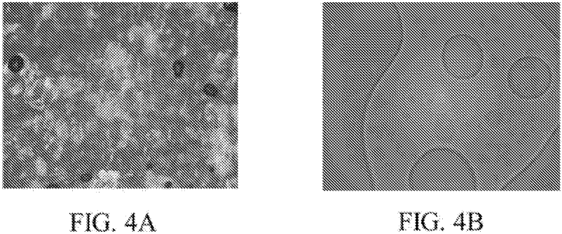

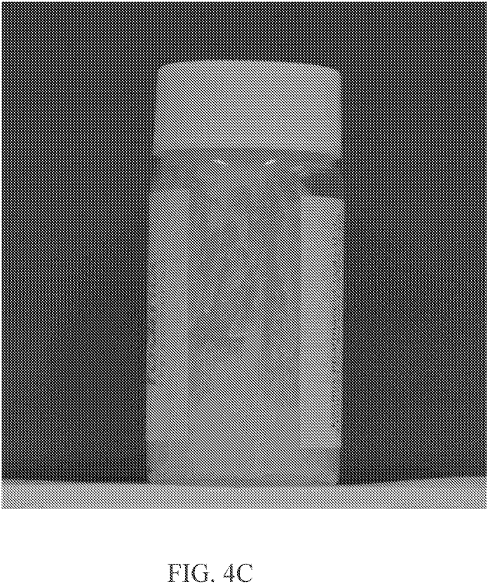

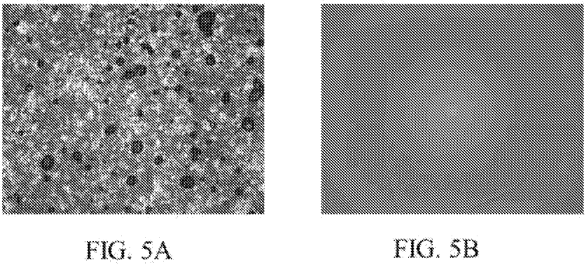

As can be seen in FIGS. 4A -4B and in Table 17A, doxycycline was dissolved in the IDD203 formulation. No grains were detected under microscopy even following 2 months incubation at 5° C., and no phase separation was observed (see FIG. 4C). A placebo formulation (without doxycycline) was also analyzed. Grains nor phase separation (see FIGS. 5A-5B) were observed, suggesting the vehicle itself is physically stable, whether or not doxycycline is present. In addition, no significant change in color and no phase separation was noted following 3 months incubation at 40° C. The color recorded following 3 months incubation at 5° C., 25° C., or 40° C. remained constant from initial preparation presenting a color score of 0.

Without being bound by any theory or mechanism of action, it is hypothesized that an organic polar phase can be distributed or entrapped in an organic apolar (or hydrophobic) phase in an essentially homogenous manner. On the basis that Doxycycline is solubilized within the organic polar phase of the composition and is not soluble in the hydrophobic phase, the concentration of doxycycline in the polar phase may be much higher than that of the entire composition. For example, if the actual effective concentration of doxycycline is 2% w/w in the composition and the amount of the polar phase in which the doxycycline is solubilized is 20% of the composition then the concentration of doxycycline in the polar phase should be 10% in the polar phase. Likewise, if the polar phase was 25% of the composition then the concentration of doxycycline in the polar phase should be 8% in the polar phase. This itself may have advantages when it comes to delivery into the skin or mucosal membrane since although the actual overall amount in the composition is much lower the polar phase may present a higher concentration to skin or mucosa and therefor a higher concentration gradient encouraging penetration. Also dissolution in the polar solvent may further assist penetration even without additional penetration enhancers.

### Example 17B -Formulations Comprising Minocycline-Glycerin in Mineral Oil and Paraffin Wax

A minocycline composition as shown in Table 17B was prepared. The composition comprises an organic polar phase (e.g., glycerol) and an organic hydrophobic phase (e.g., hydrocarbon ingredients such as a mineral oil (MO) and a paraffin wax (PW)). An exemplary composition entitled 10A is presented herein below in Table 17B.

TABLE 17B

exemplary solubilized minocycline composition

| Formulation | 10A |
|---|---|
| Ingredient | %w/w |
| Mineral oil (MO) | 38.87 |
| Paraffin wax (PW) 57-60 | 40.00 |
| Glycerin | 20.00 |
| Minocycline HCL | 1.13 |
| Total | 100.00 |
| Ratio PW:Glycerin | 2:1 |
| Ratio PW: MO | 1.03:1 |
| Ratio MO: Glycerin | 1.94:1 |
| Phase separation | NO |
| Grainy | NO |

*PW: paraffin wax; MO: Mineral oil

Similar to doxycycline, minocycline was dissolved in the 10A formulation. No grains were detected under microscopy. No phase separation was observed.

Contrary to solubilized doxycycline that was found to be stable in the herein described formulations, solubilized minocycline quickly degraded. It is speculated that the herein described compositions may be less suitable for minocycline. Thus, in one or more embodiments, the herein described composition is free or substantially free or essentially free of minocycline.

### Example 18 - Stable Formulations Comprising Doxycycline-Glycerin in Mineral Oil and Paraffin Wax

The stability of doxycycline solubilized in glycerin, mixed with mineral oil and paraffin wax, was evaluated. Formulation IDD203 was used for the study. A glycerin solution comprising doxycycline was mixed with paraffin wax 57-60 (40%w/w) and mineral oil (to 100%). Samples were incubated for 3 months at 5° C., 25° C., or 40° C. (under dark conditions). After incubation, doxycycline content (DOX assay) and degradant (4-epi) contents (%w/w) were evaluated by UPLC.

TABLE 18A

doxycycline content (DOX assay, w/w%) in formulation IDD203

| Temp. condition | Time zero | 2 weeks | 1 month | 2 months | 3 months | 4 months | 6 months |
|---|---|---|---|---|---|---|---|
| 5° C. | 97.7 | - | 96.3 | 94.7 | 95.2 | 93.1 | 95.7 |
| 25° C. | 97.7 | - | 95.7 | 96.4 | 94.6 | 92.6 | 94.2 |
| 40° C. | 97.7 | 93.0 | 93.3 | 94.2 | 93.5 | 90.6 | 91.6 |

TABLE 18B

4-epidoxycycline content (w/w %) in formulation IDD203

| Temp. condition | Time zero | 2 weeks | 1 month | 2 months | 3 months | 4 months | 6 months |
|---|---|---|---|---|---|---|---|
| 5° C. | <0.10 | - | <0.10 | <0.10 | <0.10 | <0.10 | <0.10 |
| 25° C. | <0.10 | - | 0.15 | 0.20 | 0.25 | 0.36 | 0.48 |
| 40° C. | <0.10 | 0.31 | 0.68 | 1.32 | 1.89 | 2.63 | 3.66 |
| Rate of 4-epi breakdown at 25° C. | <0.10 | - | 0.15 per month | 0.1 per month | 0.08 per month | 0.09 per month | 0.08 per month |
| Rate of 4-epi breakdown at 40° C. | <0.10 | 0.15 per week | 0.68 per month | 0.66 per month | 0.63 per month | 0.66 per month | 0.61 per month |

As can be seen in Tables 18A and 18B and in the graphs shown in FIGS. 6A-6B, IDD203 showed an excellent chemical stability at 5° C., 25° C. and 40° C.

TABLE 18C

comparison of breakdown of doxycycline in glycerin alone (composition 1 shown in Example 14) and glycerin entrapped in a hydrophobic oil diluted wax (composition 203)

| | | | | | | |
|---|---|---|---|---|---|---|
| Time (months) | 0 | 1 | 2 | 3 | 5 | 6 |
| 4-Epi (w/w%), 40° C. (composition 1) | 0.00 | 0.73 | 1.45 | 2.45 | 3.52 | 4.35 |
| 4-Epi (w/w%), 40° C. (IDD203) | 0.00 | 0.68 | 1.32 | 1.89 | 2.63 | 3.66 |

As can be seen in Table 18C, the level of 4-epimer in Composition 1 after 6 months at 40° C. was 4.35% compared to 3.66% in composition IDD203, showing a relative improvement of 19% for formulation IDD203 over Composition 1. The results shown in Table 18C are also depicted as a dot plot in FIG. 7.

Surprisingly, it was observed that the rate of doxycycline breakdown measured by the 4-Epi accumulation over a sixth month period was substantially higher in glycerin alone (composition 1) as compared to when the glycerin is entrapped in oil diluted wax (IDD203).

In one or more embodiments there is provided a method of stabilizing doxycycline in a glycerol composition, wherein the composition is a metal salt free, anti-oxidant free composition, wherein the concentration of doxycycline in glycerol is from 1 to 25%, and the concentration of doxycycline in the total composition is from about 0.1 to 5%. It is unexpected that improved stability of doxycycline is observed when it is concentrated in glycerol in the two-phase compositions described herein. In one or more embodiments, the doxycycline is more stable in the two-phase compositions described herein than it is in a single-phase polar-solvent based composition. In one or more embodiments, the doxycycline solubilized in the polar phase of the two-phase compositions described herein is more stable than a doxycycline solubilized in a single-phase polar-solvent based composition. In one or more embodiments, the two-phase compositions described herein retain more of the doxycycline initially present in the composition as compared to the amount of doxycycline retained in a single-phase polar-solvent based composition. Without being bound by any theory, it is possible that the higher stability of doxycycline in the two-phase compositions described herein is at least in part due to stabilization of the doxycycline in the interphase between the polar and hydrophobic phases. In one or more embodiments, provided herein are two-phase homogenous compositions comprising a first phase being a polar phase and a second phase; wherein an active agent (e.g., doxycycline) is solubilized in the first polar phase; wherein the first polar phase is dispersed in the second phase and wherein the active agent remains stable in the composition. In one or more embodiments, provided herein are two-phase homogenous compositions comprising a polar phase and a hydrophobic phase; wherein an active agent (e.g., doxycycline) is solubilized in the polar phase; wherein the polar phase is dispersed in the hydrophobic phase and wherein the active agent remains stable in the composition. In one or more embodiments, provided herein are methods of stabilizing an active agent in a two-phase homogenous composition comprising a polar phase and a hydrophobic phase; wherein the active agent (e.g. doxycycline) is solubilized in the polar phase and wherein the polar phase is dispersed in or entrapped within the hydrophobic phase.

### Example 19 - Foamable Composition Comprising Mineral Oil, Paraffin Wax and Glycerin

The composition shown in Table 19 comprising mineral oil (67.67 %w/w), paraffin wax 57-60 (10 %w/w), and glycerin (20 %w/w), and further doxycycline (2.33 %w/w) was prepared. The foamability of the composition was evaluated. The composition was introduced into a pressurized canister, which was then crimped. A propellant was added. Upon release of the composition, an excellent foam was produced, having a collapse time of above 180 seconds at 36° C. temperature, indicating that the foam is thermally stable.

TABLE 19

a foamable composition comprising glycerin, paraffin wax and mineral oil

| | |
|---|---|
| Formulation Ingredient (%w/w) | IDD263 |
| Mineral oil | 67.67 |
| Paraffin wax 57-60 | 10.00 |
| Glycerin | 20.00 |
| Dox | 2.33 |
| Total | 100.00 |
| Propellant (AP-70) | 12 |
| Ratio PW:MO | 0.15:1 |
| Ratio PW: glycerin | 2:1 |
| Ratio MO+PW:Glycerin | 3.88:1 |
| Phase separation | NO |
| Grain | NO |
| Foam quality | Excellent |
| Shakability | 2 |
| Density | 0.26 g/mL |
| Collapse time | >180 sec |
| Time for foam quality to change to fairly good | >180 sec |

### Example 20 - Skin Permeability of IDD203

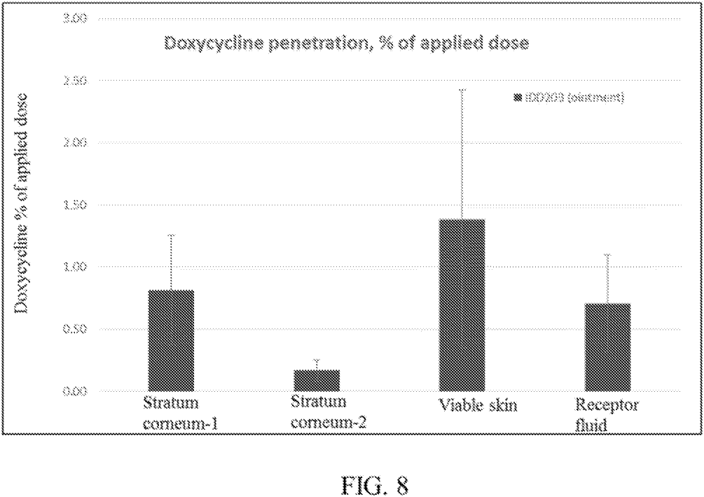

To evaluate the degree of skin penetration and permeation of IDD203, a skin penetration experiment was conducted. About 250 mg of IDD203 formulation (comprising about 5 mg of doxycycline, an excess dose higher than can typically be expected to be absorbed through skin) was applied onto portions of porcine ear skin for 24 hours using Franz diffusion cells. Following incubation, the skin pieces were stripped and the API amount was determined in each skin layer (Stratum comeum-1 external layer, stratum corneum-2 internal layer, and viable skin containing the epidermis and dermis) and in the receiver compartment (designated as Receptor fluid) using UPLC. As can be seen in FIG. 8, IDD203 presented a good level of penetration into skin viable layers and comparably reduced levels of penetration into the receiver compartment indicating the composition mainly penetrated into the skin viable layer, but not through the skin.

### Example 21 - Formulations Containing Thickened Silicones

Doxycycline formulations comprising a hydrophobic phase comprising a hydrophobic solvent, a wax and a thickened silicone and a polar phase comprising a triol were prepared. Formulations were assessed for homogeneity and grainy texture on the day of formulation manufacturing. (Table 21A).

Doxycycline formulations comprising a hydrophobic phase comprising a wax and a thickened silicone and a polar phase comprising a triol were prepared. Formulations were assessed for homogeneity and grains 72 hours after preparation at room temperature. (Table 21B).

TABLE 21A

formulations containing a mineral oil, octyldodecanol, caprylic/capric triglycerides, a microcrystalline wax, a paraffin wax a thickened silicone and a glycerin

| | | | | | |
|---|---|---|---|---|---|
| | IDD342 | IDD343 | IDD344 | IDD345 | IDD346 |
| Mineral oil (MO) | 8.83 | 8.83 | 8.83 | 8.83 | 13.83 |
| Glycerin | 10.00 | 10.00 | 10.00 | 10.00 | 10.00 |
| Microcrystalline wax (76.7-82.2) | 20.00 | 20.00 | 20.00 | 20.00 | 15.00 |
| Paraffin wax (57-60) | 10.00 | 10.00 | 10.00 | 10.00 | 10.00 |
| MGS-Elastomer 1100 | 20.00 | | | | 20.00 |
| Gransil DMG5 | | 20.00 | | | |
| MGS powder 3300 | | | 20.00 | | |
| MGS-Elastomer 1148P | | | | 20.00 | |
| Octyldodecanol (OCT) | 10.00 | 10.00 | 10.00 | 10.00 | 10.00 |
| Cyclomethicone | | | | | |
| Caprylic/Capric Triglycerides (MCT) | 20.00 | 20.00 | 20.00 | 20.00 | 20.00 |
| Doxycycline hyclate | 1.17 | 1.17 | 1.17 | 1.17 | 1.17 |
| Total | 100.00 | 100.00 | 100.00 | 100.00 | 100.00 |
| Ratio PW+MW:Glycerin | 3:1 | 3:1 | 3:1 | 3:1 | 2.5:1 |
| Ratio PW+MW: MO+OCT+MCT | 1:1.3 | 1:1.3 | 1:1.3 | 1:1.3 | 1:1.5 |
| MO+OCT+MCT:glycerin | 3.88:1 | 3.88:1 | 3.88:1 | 3.88:1 | 4.4:1 |
| Ratio PW+MW+MO+OCT+MCT:glycerin | 6.88:1 | 6.88:1 | 6.88:1 | 6.88:1 | 6.88:1 |
| Dox: Glycerin | 1:8.55 | 1:8.55 | 1:8.55 | 1:8.55 | 1:8.55 |
| Thickened silicone:glycerin | 2:1 | 2:1 | 2:1 | 2:1 | 2:1 |
| Phase separation | NO | NO | NO | NO | NO |
| Grainy | NO | NO | NO | NO | NO |

TABLE 21A Cont.

formulations containing a mineral oil, octyldodecanol, caprylic/capric triglycerides, a microcrystalline wax, a paraffin wax a thickened silicone and a glycerin

| | | | | |
|---|---|---|---|---|
| | IDD347 | IDD348 | IDD349 | 16B |
| Mineral oil (MO) | 13.83 | 13.83 | 13.83 | 20.00 |
| Glycerin | 10.00 | 10.00 | 10.00 | 20.00 |
| Microcrystalline wax (76.7-82.2) | 15.00 | 15.00 | 15.00 | |
| Paraffin wax (57-60) | 10.00 | 10.00 | 10.00 | 40.00 |
| MGS-Elastomer 1100 | | | | |
| Gransil DMG5 | 20.00 | | | |
| MGS powder 3300 | | 20.00 | | |
| MGS-Elastomer 1148P | | | 20.00 | |
| Octyldodecanol (OCT) | 10.00 | 10.00 | 10.00 | |
| Cyclomethicone | | | | 17.67 |
| Caprylic/Capric Triglycerides (MCT) | 20.00 | 20.00 | 20.00 | |
| Doxycycline hyclate | 1.17 | 1.17 | 1.17 | 2.33 |
| Total | 100.00 | 100.00 | 100.00 | 100.00 |
| Ratio PW+MW:Glycerin | 2.5:1 | 2.51:1 | 2.5:1 | 2:1* |
| Ratio PW+MW: MO+OCT+MCT | 1:1.5 | 1:1.5 | 1:1.5 | 1.06:1** |
| MO+OCT+MCT:glycerin | 4.4:1 | 4.4:1 | 4.4:1 | 1.88:1^{#} |
| Ratio PW+MW+MO+OCT+MCT:glycerin | 6.88:1 | 6.88:1 | 6.88:1 | 3.88:1^{##} |
| Dox: Glycerin | 1:8.55 | 1:8.55 | 1:8.55 | 1:8.58 |
| Thickened silicone:glycerin | 2:1 | 2:1 | 2:1 | N/A |
| Phase separation | NO | NO | NO | NO |
| Grainy | NO | NO | NO | YES |

*Ratio PW:Glycerin;
** Ratio PW:MO+Cyclomethicone;
# Ratio MO+Cyclomethicone:Glycerin;
## Ratio PW+MO+Cyclomethicone:Glycerin

As can be seen from table 21A all the compositions comprising a thickened silicone were homogenous and smooth (not grainy), compared to formulation 16B containing cyclomethicone that was homogenous but grainy. In one or more embodiments, the compositions comprising a thickened silicone have improved skin feeling.

TABLE 21B

formulations containing a thickened silicone, a glycerin and a microcrystalline wax or a paraffin wax

| Formulation | IDD352 | IDD353 | IDD354 | IDD355 | IDD356 |
|---|---|---|---|---|---|
| Ingredient | %w/w | %w/w | %w/w | %w/w | %w/w |
| Glycerin | 10.00 | 10.00 | 10.00 | 10.00 | 10.00 |
| Microcrystalline wax (76.7-82.2) | 10.00 | 10.00 | 20.00 | 20.00 | |
| Paraffin wax (57-60) | | | | | 10.00 |
| Gransil DMG5 | 78.83 | | 68.83 | | 78.83 |
| ST-Elastomer 10 | | 78.83 | | 68.83 | |
| Doxycycline hyclate | 1.17 | 1.17 | 1.17 | 1.17 | 1.17 |
| Total | 100.00 | 100.00 | 100.00 | 100.00 | 100.00 |
| Ratio PW/MW:Glycerin | 1:1 | 1:1 | 2:1 | 2:1 | 1:1 |
| Ratio PW/MW + Thickened silicone: Glycerin | 8.9:1 | 8.9:1 | 8.9:1 | 8.9:1 | 8.9:1 |
| Dox:Glycerin | 1:8.55 | 1:8.55 | 1:8.55 | 1:8.55 | 1:8.55 |
| Thickened silicone:glycerin | 7.9:1 | 7.9:1 | 6.9:1 | 6.9:1 | 7.9:1 |
| Phase separation | NO | NO | NO | NO | NO |
| Grainy | NO | NO | NO | NO | NO |

PW = paraffin wax; MW=microcrystalline wax

TABLE 21B Cont.

formulations containing a thickened silicone, a glycerin and a microcrystalline wax or a paraffin wax

| Formulation | IDD357 | IDD358 | IDD359 | IDD360 | IDD361 |
|---|---|---|---|---|---|
| Ingredient | %w/w | %w/w | %w/w | %w/w | %w/w |
| Glycerin | 10.00 | 10.00 | 10.00 | 20.00 | 20.00 |
| Microcrystalline wax (76.7-82.2) | | | | 10.00 | 10.00 |
| Paraffin wax (57-60) | 10.00 | 20.00 | 20.00 | | |
| Gransil DMG5 | | 68.83 | | 68.83 | |
| ST-Elastomer 10 | 78.83 | | 68.83 | | 68.83 |
| Doxycycline hyclate | 1.17 | 1.17 | 1.17 | 1.17 | 1.17 |
| Total | 100.00 | 100.00 | 100.00 | 100.00 | 100.00 |
| Ratio PW/MW:Glycerin | 1:1 | 2:1 | 2:1 | 0.5:1 | 0.5:1 |
| Ratio PW/MW + Thickened silicone: Glycerin | 8.9:1 | 8.9:1 | 8.9:1 | 3.9:1 | 3.9:1 |
| Dox:Glycerin | 1:8.55 | 1:8.55 | 1:8.55 | 1:17.1 | 1:17.1 |
| Thickened silicone:glycerin | 7.9:1 | 6.9:1 | 6.9:1 | 3.44:1 | 3.44: 1 |
| Phase separation | NO | NO | NO | NO | NO |
| Grainy | NO | NO | NO | NO | NO |

As can be seen from Table 21B all the compositions comprising a thickened silicone were homogenous and smooth (not grainy).

### Example 22 - a Formulation With an Emulsifying Wax

A doxycycline formulation including a hydrophobic phase comprising a hydrophobic solvent (an oil) and a wax, and a polar phase comprising a triol was prepared.

TABLE 22

a formulation comprising a mineral oil, an emulsifying wax and glycerin

| Formulation | 2C |
|---|---|
| Ingredient | %w/w |
| Mineral oil | 37.67 |
| Emulsifying wax | 40.00 |
| Glycerin | 20.00 |
| Dox | 2.33 |
| Total | 100.00 |
| Ratio Wax:glycerin | 2:1 |
| Ratio wax:MO | 1.06:1 |
| Ratio MO: glycerin | 1.88:1 |
| Ratio wax+MO:glycerin | 3.88:1 |

MO = mineral oil

Formulation 2C produced a homogenous and smooth (not grainy) composition.

Thus, in some embodiments, the liquisoft composition comprises an oil, a wax and a polar solvent, wherein the wax comprises an emulsifying wax. In other embodiments, the composition is free, essentially free or substantially free of emulsifying wax.

### Example 23 - Compatibility Studies

The following excipients are compatible with minocycline HCL: Aerosil, Almond oil, Avocado oil, Beeswax, Behenyl alcohol, Calendula oil, Castor oil, Cetearyl octanoate, Cetostearyl alcohol, Cetyl alcohol, Cocoglycerides, Coconut oil, Cyclomethicone 5 NF, Diisopropyl adipate, Gelled mineral oil, Grape seed oil, Hard Fat, Hydrogenated Castor Oil, Isododecane, isopropyl myristate, Isostearyl alcohol, Jojoba oil, MCT Oil, Menthol, Mineral Oil, Myristyl alcohol, Octyldodecanol, Oleic acid, Oleyl alcohol, Palmitic acid, Paraffin 51-53, Paraffin 51-53, Paraffin 58-62, Peanut oil, Petrolatum (sofmetic), PPG-15 Stearyl Ether, Shea butter, Stearic acid, Stearyl alcohol, Sucrose stearic acid esters D1803, Sucrose stearic acid esters D1807, Sucrose stearic acid esters D1811, Wheat germ oil, White petrolatum. See U.S. 8,343,945 and U.S. 8,945,516, which are incorporated herein by reference with respect to the excipients and their compatibility described herein and therein.

Additional excipients compatible with minocycline HCL are: soybean oil, paraffin 57-64, polyvinyl alcohol and hydroxypropylcellulose (klucel).

The following excipients are compatible with doxycycline hyclate:

Cetearyl octanoate, Cyclomethicone 5 NF, Diisopropyl adipate, Ethanol 95%, Hexylene glycol, Hydrogenated Castor Oil, Isostearic acid, MCT Oil, Mineral Oil, Myristyl alcohol, Oleyl alcohol, PEG 100 Stearate, PPG-15 Stearyl Ether, Sorbitan Monostearate (SPAN 60), Stearyl alcohol. See U.S. 8,343,945, which is incorporated here by reference with respect to the excipients and their compatibility described herein and therein.

### Example 24 - Prophetic Formulation

Doxycycline formulation including a hydrophobic phase comprising a hydrophobic solvent (a liquid fatty alcohol) and a wax, and a polar phase comprising a triol is described.

TABLE 23

a formulation comprising isostearyl alcohol, paraffin wax and glycerin

| Formulation | 15A |
|---|---|
| Ingredient | %w/w |
| Isostearyl alcohol (LFA) | 37.67 |
| Paraffin wax 57-60 | 40.00 |
| Glycerin | 20.00 |
| Dox | 2.33 |
| Total | 100.00 |
| Ratio Wax:glycerin | 2:1 |
| Ratio wax:LFA | 1.06:1 |
| Ratio LFA: glycerin | 1.88:1 |
| Ratio wax+ LFA:glycerin | 3.88:1 |

LFA=liquid fatty alcohol

Thus, in one or more embodiments, the hydrophobic phase may include a liquid fatty alcohol. In one or more embodiments, the hydrophobic phase may include a liquid fatty alcohol comprising one or more of capryl alcohol, 1-nonanol, undecanol, isostearyl alcohol and oleyl alcohol.

What is claimed is:

1. A liquisoft complex composition comprising:
a. a polar phase comprising a dissolved doxycycline;
b. a flowable or semi-solid hydrophobic phase;
wherein the polar phase is pseudo-dispersed homogeneously within the hydrophobic phase without a surfactant to interlink and stabilize the hydrophobic and polar phases.

2. The liquisoft composition of claim 1, wherein the hydrophobic phase forms an entrapment framework and the polar phase is substantially entrapped within the entrapment framework, wherein the entrapment framework comprises a wax or combinations of two or more waxes having a melting point above 37° C. and a hydrophobic solvent.

3. The liquisoft composition of claim 2, wherein the wax comprises one or more of a paraffin wax, a microcrystalline wax, a beeswax, and a hydrogenated oil, and wherein the hydrophobic solvent comprises an oil that is liquid at 25° C.

4. The liquisoft composition of claim 3, wherein the polar phase comprises a polyol, wherein the polyol comprises a triol, a triol and a diol, or a triol and a monohydric alcohol, and wherein the oil comprises one or more of a mineral oil, a plant oil, an oil rich in triglycerides, a medium chain triglyceride oil, and/or a soybean oil.

5. The liquisoft composition of claim 4, wherein the triol comprises glycerol; wherein the diol, if present, comprises a glycol; and wherein the monohydric alcohol, if present, comprises a short chain alcohol with a C1 to C6 carbon chain.

6. The liquisoft composition of claim 5, wherein the ratio of wax to polyol is about or more than 0.5:1.

7. The liquisoft composition of claim 6, wherein the ratio of hydrophobic phase to polar phase is about 1:1: to about 5:1.

8. The liquisoft composition of claim 7, wherein the hydrophobic phase further comprises a petrolatum, a silicone oil, a thickened silicone or mixtures of any two or more thereof.

9. The liquisoft composition of claim 5, wherein the polar phase comprises up to about 35% by weight of the composition of a polyol; and the hydrophobic phase comprises a) about 5% to about 45% by weight of the composition of at least one wax; and b) about 30% to about 75% by weight of the composition of at least one hydrophobic liquid solvent.

10. The liquisoft composition of claims 9, wherein the polar phase comprises about 15% to about 25% by weight of the composition of a polyol; and the hydrophobic phase comprises a) about 18% to about 42% by weight of the composition of at least one wax; and b) about 35% to about 63% by weight of the composition of at least one hydrophobic liquid solvent.

11. The liquisoft composition of claim 9, wherein the composition is substantially free of water other than water that is associated with the active pharmaceutical agent.

12. The liquisoft composition of claim 10, wherein the composition is gel- or ointment-like and when mixed with a liquefied or a compressed gas propellant in a canister can form a foam upon release, wherein the ratio of composition to propellant is between about 100:4 to about 100:25.

13. The liquisoft composition of claim 5, wherein the concentration of doxycycline in the polar phase is higher than in the composition.

14. The liquisoft composition of claim 13, wherein the stability of doxycycline in the polar solvent in the composition is improved compared to its stability in the polar solvent alone.

15. The liquisoft composition of claim 14, wherein when stored at 25° C. for three months, the composition retains above about 90% of the doxycycline initially present in the composition and less than about 0.9% breaks down to 4-epi; or wherein when stored at 5° C. for six months, the composition retains above about 90% of the doxycycline initially present in the composition and less than about 1.8% breaks down to 4-epi.

16. The liquisoft composition of claim 11, wherein the doxycycline is doxycycline hyclate and is present in an effective pharmaceutical concentration for topical application to the skin or mucosa to treat a skin disorder involving a bacterial infection and or inflammation.

17. The liquisoft composition of claim 16, wherein the concentration of doxycycline hyclate is about 0.5% to about 3.5% in the composition and is about 1.4% to about 10% in the polar phase.

18. The liquisoft composition of claim 17, wherein the disorder comprises an acne or a rosacea.

19. The liquisoft composition of claim 5, for use in the treatment of a dermatological, an ophthalmological, a gynecological, or a mucosal disease or disorder, or its use in the manufacture of a medicament having activity for the treatment thereof.

20. A method of stabilizing or increasing stability of a doxycycline, comprising:
adding the doxycycline to the polar phase in the liquisoft composition of claim 5 at a concentration in which the doxycycline is soluble but below the concentration in which it recrystalizes at 25° C.;
dissolving the doxycycline in the polar phase;
mixing the polar phase comprising the dissolved doxycycline with the hydrophobic phase at a temperature at which the hydrophobic phase is liquid; and
cooling with mixing to form a substantially homogenous composition;
wherein the concentration of doxycycline in the polar phase is at least twice than that in the composition; and wherein its stability in the composition is higher than the stability of the same concentration of doxycycline in an equivalent polar phase that is not pseudo-dispersed within the
hydrophobic phase.

21. The method of claim 20, wherein the doxycycline is doxycycline hyclate.

22. A method of treating or alleviating the symptoms of a dermatological, an ophthalmological, a gynecological, or a mucosal disorder, comprising: applying to a target area the liquisoft composition of claim 5, wherein the disorder includes at least one etiological factor selected from the group consisting of an infection, an inflammation, oxidative stress, neurodegeneration, and apoptosis.

23. The method of claim 22, wherein the disorder is an inflammation of skin, mucosa or eye and/or wherein the disorder is an infection of skin, mucosa or eye, wherein the polar phase comprises about 15% to about 25% by weight of the composition; and wherein the hydrophobic phase comprises a) about 35% to about 45% by weight of the composition of at least one wax; and b) about 20% to about 50% by weight of the composition of at least one hydrophobic liquid solvent.

24. The method of claim 22, wherein the disorder is an acne or a rosacea.

25. The method of claim 24, wherein the disorder is an acne.

26. The method of claim 24, wherein the disorder is a rosacea.
